SEQUENCE LISTING (45 sequences)

SEQ ID NO: 1 - DNA, 3349 bp, Artificial Sequence
  Artificial polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 2 - DNA, 3295 bp, Artificial Sequence
  Artificial polynucleotide
ggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaatacggttcctgcctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgcccgtcagacccgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttacc

SEQ ID NO: 3 - DNA, 3592 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaatacggttcctgcctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaacgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttcgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgcccgtcagacccgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 4 - DNA, 3405 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaatacggttcctgcctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaacgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgcccgtcagacccgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 5 - DNA, 5472 bp, Artificial Sequence
  Artificial polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 6 - DNA, 35 bp, Artificial Sequence
  Artificial polynucleotide
gacccgggcggccgcttccctttagtgagggttaa

SEQ ID NO: 7 - DNA, 42 bp, Artificial Sequence
  Artificial polynucleotide
tgctgccactccgtgtaccacatttgtagaggttttacttgc

SEQ ID NO: 8 - DNA, 42 bp, Artificial Sequence
  Artificial polynucleotide
gtggtacacggagtggcagcaccatggcctgaaataacctct

SEQ ID NO: 9 - DNA, 33 bp, Artificial Sequence
  Artificial polynucleotide
caaaagcctaggcctccaaaaaagcctcctcac

SEQ ID NO: 10 - DNA, 4852 bp, Artificial Sequence
  Artificial polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 11 - DNA, 30 bp, Artificial Sequence
  Artificial polynucleotide
ggaggcctaggcttttgcaaaaagctgagc

SEQ ID NO: 12 - DNA, 55 bp, Artificial Sequence
  Artificial polynucleotide
tcgtattatactatgccgatatactatgccgatgattaattgtcaacacgtgctg

SEQ ID NO: 13 - DNA, 55 bp, Artificial Sequence
  Artificial polynucleotide
cagcacgtgttgacaattaatcatcggcatagtatatcggcatagtataatacga

SEQ ID NO: 14 - DNA, 37 bp, Artificial Sequence
  Artificial polynucleotide
tcgacggtatacagacatgataagatacattgatgag

SEQ ID NO: 15 - DNA, 4357 bp, Artificial Sequence
  Artificial polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 16 - DNA, 60 bp, Artificial Sequence
  Artificial polynucleotide
tgagtttcatgaggcgcgcccgtcagacccgtttaaacagatcaaaggatcttcttgaga

SEQ ID NO: 17 - DNA, 60 bp, Artificial Sequence
  Artificial polynucleotide
tattgaagatctcctgcaggcaggaaccgtatttaaatcgcgttgctggcgtttttccat

SEQ ID NO: 18 - DNA, 31 bp, Artificial Sequence
  Artificial polynucleotide
tttggaggcctaggcttttgcaaaaagctcc

SEQ ID NO: 19 - DNA, 57 bp, Artificial Sequence
  Artificial polynucleotide
gaggcgcaccgtgggcttgtactcggtcatggtggcgtttagttcctcaccttgtcg

SEQ ID NO: 20 - DNA, 57 bp, Artificial Sequence
  Artificial polynucleotide
cgacaaggtgaggaactaaacgccaccatgaccgagtacaagcccacggtgcgcctc

SEQ ID NO: 21 - DNA, 34 bp, Artificial Sequence
  Artificial polynucleotide
catccagccggctcaggcaccgggcttgcgggtc

SEQ ID NO: 22 - DNA, 31 bp, Artificial Sequence; identical to SEQ ID NO: 18
  Artificial polynucleotide

SEQ ID NO: 23 - DNA, 59 bp, Artificial Sequence
  Artificial polynucleotide
gtgcaatccatcttgttcaatcatggtggcgttcctcaccttgtcgtattatactatgc

SEQ ID NO: 24 - DNA, 59 bp, Artificial Sequence
  Artificial polynucleotide
gcatagtataatacgacaaggtgaggaacgccaccatgattgaacaagatggattgcac

SEQ ID NO: 25 - DNA, 34 bp, Artificial Sequence; identical to SEQ ID NO: 21
  Artificial polynucleotide

SEQ ID NO: 26 - DNA, 60 bp, Artificial Sequence
  Artificial polynucleotide
agcctcgagaattctaataggccaccatgtccactgcggtcctggaaaacccaggcttgg

SEQ ID NO: 27 - DNA, 58 bp, Artificial Sequence
  Artificial polynucleotide
ggaagcggccgcctactttattttctggagggcactgcaaaggattccaatttcactg

SEQ ID NO: 28 - DNA, 4642 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaatacggttcctgcctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctaataggccaccatgtccactgcggtcctggaaaacccaggcttgggcaggaaactctctgactttggacaggaaacaagctatattgaagacaactgcaatcaaaatggtgccatatcgctgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaaggaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgcccgtcagacccgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 29 - DNA, 6473 bp, Artificial Sequence
  Artificial polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctaataggccaccatgtccactgcggtcctggaaaacccaggcttgggcaggaaactctctgactttggacaggaaacaagctatattgaagacaactgcaatcaaaatggtgccatatcgctgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaaggaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaggcggccgctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 30 - DNA, 60 bp, Artificial Sequence
  Artifical polynucleotide
aggctagcctcgaggtaataggccaccatgcagatcgagctgtccacctgcttttttctg

SEQ ID NO: 31 - DNA, 60 bp, Artificial Sequence
  Artificial polynucleotide
cagggttgtccgggtgatctcccgctggtgacgcgtgctggacacattcttgccccagct

SEQ ID NO: 32 - DNA, 60 bp, Artificial Sequence
  Artificial polynucleotide
agctggggcaagaatgtgtccagcacgcgtcaccagcgggagatcacccggacaaccctg

SEQ ID NO: 33 - DNA, 60 bp, Artificial Sequence
  Artificial polynucleotide
ggaagcggccgctcatcagtacagatcctgggcctcacatcccaggacttccatcctgag

SEQ ID NO: 34 - DNA, 8340 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaatacggttcctgcctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtaataggccaccatgcagatcgagctgtccacctgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaatatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgggagaaagaagatgacaaggtgttccctggcggcagccacacctacgtgtggcaggtgctgaaagaaaacggccccatggcctccgaccccctgtgcctgacctacagctacctgagccacgtggacctggtgaaggacctgaacagcggcctgatcggcgctctgctcgtctgccgggagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtccggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcttgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactatatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagacattcaagacccgggaggccatccagcacgagagcggcatcctgggccccctgctgtacggcgaagtgggcgacacactgctgatcatcttcaagaaccaggctagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggcgtgcagctggaagatcccgagttccaggccagcaacatcatgcactccatcaatggctacgtgttcgactctctgcagctctccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagacagtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgaaccccggagcttcagccagaacccccccgtgctgacgcgtagcttcagccagaacagccggcaccccagcacccggcagaagcagttcaacgccaccaccatccccgagaacgacatcgagaaaaccgacccttggtttgcccaccggacccccatgcccaagatccagaacgtgtccagcagcgacctgctgatgctgctgcggcagagccccacccctcacggcctgagcctgagcgacctgcaggaagccaagtacgagacattcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaggtctccagcaccagcaacaacctgatcagcaccatccccagcgacaacctggccgctggcaccgacaacaccagcagcctgggccctcccagcatgcccgtgcactacgacagccagctggacaccaccctgttcggcaagaagtccagccccctgaccgagtccggcggacccctgtccctgagcgaggaaaacaacgacagcaagctgctggaaagcggcctgatgaacagccaggaaagcagctggggcaagaatgtgtccagcacgcgtcaccagcgggagatcacccggacaaccctgcagtccgaccaggaagagatcgattacgacgacaccatcagcgtggagatgaagaaagaggatttcgatatctacgacgaggacgagaaccagagccccagaagcttccagaagaaaacccggcactacttcattgccgccgtggagaggctgtgggactacggcatgagttctagcccccacgtgctgcggaaccgggcccagagcggcagcgtgccccagttcaagaaagtggtgttccaggaattcacagacggcagcttcacccagcctctgtatagaggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgccgaaccccggaagaacttcgtgaagcccaacgaaaccaagacctacttctggaaagtgcagcaccacatggcccccaccaaggacgagttcgactgcaaggcctgggcctacttcagcgacgtggatctggaaaaggacgtgcactctggactgattggcccactcctggtctgccacactaacaccctcaaccccgcccacggccgccaggtgaccgtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagagaatccggtggtatctgctgtccatgggcagcaacgagaatatccacagcatccacttcagcggccacgtgttcaccgtgcggaagaaagaagagtacaagatggccctgtacaacctgtaccccggcgtgttcgagacagtggagatgctgcccagcaaggccggcatctggcgggtggagtgtctgatcggcgagcacctgcacgctggcatgagcaccctgtttctggtgtacagcaacaagtgccagaccccactgggcatggcctctggccacatccgggacttccagatcaccgcctccggccagtacggccagtgggcccccaagctggccagactgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaatgtggacagcagcggcatcaagcacaacatcttcaacccccccatcattgcccggtacatccggctgcaccccacccactacagcattagatccacactgagaatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtcaacaaccccaaagaatggctgcaggtggactttcagaaaaccatgaaggtgaccggcgtgaccacccagggcgtgaaaagcctgctgaccagcatgtacgtgaaagagtttctgatcagcagctctcaggatggccaccagtggaccctgttctttcagaacggcaaggtgaaagtgttccagggcaaccaggactccttcacccccgtggtgaactccctggacccccccctgctgacccgctacctgagaatccacccccagtcttgggtgcaccagatcgccctcaggatggaagtcctgggatgtgaggcccaggatctgtactgatgagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgcccgtcagacccgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 35 - DNA, 10141 bp, Artificial Sequence
  Artificial polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgtcgaggtaataggccaccatgcagatcgagctgtccacctgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaatatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgggagaaagaagatgacaaggtgttccctggcggcagccacacctacgtgtggcaggtgctgaaagaaaacggccccatggcctccgaccccctgtgcctgacctacagctacctgagccacgtggacctggtgaaggacctgaacagcggcctgatcggcgctctgctcgtctgccgggagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtccggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcttgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactatatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagacattcaagacccgggaggccatccagcacgagagcggcatcctgggccccctgctgtacggcgaagtgggcgacacactgctgatcatcttcaagaaccaggctagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggcgtgcagctggaagatcccgagttccaggccagcaacatcatgcactccatcaatggctacgtgttcgactctctgcagctctccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagacagtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgaaccccggagcttcagccagaacccccccgtgctgacgcgtagcttcagccagaacagccggcaccccagcacccggcagaagcagttcaacgccaccaccatccccgagaacgacatcgagaaaaccgacccttggtttgcccaccggacccccatgcccaagatccagaacgtgtccagcagcgacctgctgatgctgctgcggcagagccccacccctcacggcctgagcctgagcgacctgcaggaagccaagtacgagacattcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaggtctccagcaccagcaacaacctgatcagcaccatccccagcgacaacctggccgctggcaccgacaacaccagcagcctgggccctcccagcatgcccgtgcactacgacagccagctggacaccaccctgttcggcaagaagtccagccccctgaccgagtccggcggacccctgtccctgagcgaggaaaacaacgacagcaagctgctggaaagcggcctgatgaacagccaggaaagcagctggggcaagaatgtgtccagcacgcgtcaccagcgggagatcacccggacaaccctgcagtccgaccaggaagagatcgattacgacgacaccatcagcgtggagatgaagaaagaggatttcgatatctacgacgaggacgagaaccagagccccagaagcttccagaagaaaacccggcactacttcattgccgccgtggagaggctgtgggactacggcatgagttctagcccccacgtgctgcggaaccgggcccagagcggcagcgtgccccagttcaagaaagtggtgttccaggaattcacagacggcagcttcacccagcctctgtatagaggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgccgaaccccggaagaacttcgtgaagcccaacgaaaccaagacctacttctggaaagtgcagcaccacatggcccccaccaaggacgagttcgactgcaaggcctgggcctacttcagcgacgtggatctggaaaaggacgtgcactctggactgattggcccactcctggtctgccacactaacaccctcaaccccgcccacggccgccaggtgaccgtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagagaatccggtggtatctgctgtccatgggcagcaacgagaatatccacagcatccacttcagcggccacgtgttcaccgtgcggaagaaagaagagtacaagatggccctgtacaacctgtaccccggcgtgttcgagacagtggagatgctgcccagcaaggccggcatctggcgggtggagtgtctgatcggcgagcacctgcacgctggcatgagcaccctgtttctggtgtacagcaacaagtgccagaccccactgggcatggcctctggccacatccgggacttccagatcaccgcctccggccagtacggccagtgggcccccaagctggccagactgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaatgtggacagcagcggcatcaagcacaacatcttcaacccccccatcattgcccggtacatccggctgcaccccacccactacagcattagatccacactgagaatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtcaacaaccccaaagaatggctgcaggtggactttcagaaaaccatgaaggtgaccggcgtgaccacccagggcgtgaaaagcctgctgaccagcatgtacgtgaaagagtttctgatcagcagctctcaggatggccaccagtggaccctgttctttcagaacggcaaggtgaaagtgttccagggcaaccaggactccttcacccccgtggtgaactccctggacccccccctgctgacccgctacctgagaatccacccccagtcttgggtgcaccagatcgccctcaggatggaagtcctgggatgtgaggcccaggatctgtactgatgagcggccgctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 36 - DNA, 46 bp, Artificial Sequence
  Artificial polynucleotide
agcaacgcgatttaaattgctttctctgaccagcattctctcccct

SEQ ID NO: 37 - DNA, 52 bp, Artificial Sequence
  Artificial polynucleotide
tgaagatctcctgcagggccccactgtggggtggaggggacagataaaagta

SEQ ID NO: 38 - DNA, 53 bp, Artificial Sequence
  Artificial polynucleotide
tactcatgaggcgcgccactactagggacaggattggtgacagaaaagcccca

SEQ ID NO: 39 - DNA, 49 bp, Artificial Sequence
  Artificial polynucleotide
tgatctgtttaaacagagcagagccaggaacacctgtagggaaggggca

SEQ ID NO: 40 - DNA, 4923 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaattgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgccactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggtgttcctggctctgctctgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 41 - DNA, 6266 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaattgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattctaataggccaccatgtccactgcggtcctggaaaacccaggcttgggcaggaaactctctgactttggacaggaaacaagctatattgaagacaactgcaatcaaaatggtgccatatcgctgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaaggaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgccactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggtgttcctggctctgctctgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 42 - DNA, 9964 bp, Artificial Sequence
  Artificial polynucleotide
tgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatttaaattgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaggagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgaggtaataggccaccatgcagatcgagctgtccacctgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaatatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgggagaaagaagatgacaaggtgttccctggcggcagccacacctacgtgtggcaggtgctgaaagaaaacggccccatggcctccgaccccctgtgcctgacctacagctacctgagccacgtggacctggtgaaggacctgaacagcggcctgatcggcgctctgctcgtctgccgggagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtccggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcttgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactatatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagacattcaagacccgggaggccatccagcacgagagcggcatcctgggccccctgctgtacggcgaagtgggcgacacactgctgatcatcttcaagaaccaggctagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggcgtgcagctggaagatcccgagttccaggccagcaacatcatgcactccatcaatggctacgtgttcgactctctgcagctctccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagacagtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgaaccccggagcttcagccagaacccccccgtgctgacgcgtagcttcagccagaacagccggcaccccagcacccggcagaagcagttcaacgccaccaccatccccgagaacgacatcgagaaaaccgacccttggtttgcccaccggacccccatgcccaagatccagaacgtgtccagcagcgacctgctgatgctgctgcggcagagccccacccctcacggcctgagcctgagcgacctgcaggaagccaagtacgagacattcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaggtctccagcaccagcaacaacctgatcagcaccatccccagcgacaacctggccgctggcaccgacaacaccagcagcctgggccctcccagcatgcccgtgcactacgacagccagctggacaccaccctgttcggcaagaagtccagccccctgaccgagtccggcggacccctgtccctgagcgaggaaaacaacgacagcaagctgctggaaagcggcctgatgaacagccaggaaagcagctggggcaagaatgtgtccagcacgcgtcaccagcgggagatcacccggacaaccctgcagtccgaccaggaagagatcgattacgacgacaccatcagcgtggagatgaagaaagaggatttcgatatctacgacgaggacgagaaccagagccccagaagcttccagaagaaaacccggcactacttcattgccgccgtggagaggctgtgggactacggcatgagttctagcccccacgtgctgcggaaccgggcccagagcggcagcgtgccccagttcaagaaagtggtgttccaggaattcacagacggcagcttcacccagcctctgtatagaggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgccgaaccccggaagaacttcgtgaagcccaacgaaaccaagacctacttctggaaagtgcagcaccacatggcccccaccaaggacgagttcgactgcaaggcctgggcctacttcagcgacgtggatctggaaaaggacgtgcactctggactgattggcccactcctggtctgccacactaacaccctcaaccccgcccacggccgccaggtgaccgtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagagaatccggtggtatctgctgtccatgggcagcaacgagaatatccacagcatccacttcagcggccacgtgttcaccgtgcggaagaaagaagagtacaagatggccctgtacaacctgtaccccggcgtgttcgagacagtggagatgctgcccagcaaggccggcatctggcgggtggagtgtctgatcggcgagcacctgcacgctggcatgagcaccctgtttctggtgtacagcaacaagtgccagaccccactgggcatggcctctggccacatccgggacttccagatcaccgcctccggccagtacggccagtgggcccccaagctggccagactgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaatgtggacagcagcggcatcaagcacaacatcttcaacccccccatcattgcccggtacatccggctgcaccccacccactacagcattagatccacactgagaatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtcaacaaccccaaagaatggctgcaggtggactttcagaaaaccatgaaggtgaccggcgtgaccacccagggcgtgaaaagcctgctgaccagcatgtacgtgaaagagtttctgatcagcagctctcaggatggccaccagtggaccctgttctttcagaacggcaaggtgaaagtgttccagggcaaccaggactccttcacccccgtggtgaactccctggacccccccctgctgacccgctacctgagaatccacccccagtcttgggtgcaccagatcgccctcaggatggaagtcctgggatgtgaggcccaggatctgtactgatgagcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtacacggagtggcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccggccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtactcatgaggcgcgccactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggtgttcctggctctgctctgtttaaacagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcg

SEQ ID NO: 43 - DNA, 8134 bp, Artificial Sequence
  Artificial polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctaataggccaccatgtccactgcggtcctggaaaacccaggcttgggcaggaaactctctgactttggacaggaaacaagctatattgaagacaactgcaatcaaaatggtgccatatcgctgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaaggaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctctattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaggcggccgctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttcccgcgccactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggtgttcctggctctgctctgttttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaataaattgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaattattgaagcatttatcagggttattgtctcatgagcggatacatatttgaatgtatttagaaaaataaacaaataggggttccgcgcacatttccccgaaaagtgccacctgacgtc

SEQ ID NO: 44 - DNA, 11805 bp, Artificial Sequence
  Artificial polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttgtcgaggtaataggccaccatgcagatcgagctgtccacctgcttttttctgtgcctgctgcggttctgcttcagcgccacccggcggtactacctgggcgccgtggagctgtcctgggactacatgcagagcgacctgggcgagctgcccgtggacgcccggttcccccccagagtgcccaagagcttccccttcaacaccagcgtggtgtacaagaaaaccctgttcgtggagttcaccgaccacctgttcaatatcgccaagcccaggcccccctggatgggcctgctgggccccaccatccaggccgaggtgtacgacaccgtggtgatcaccctgaagaacatggccagccaccccgtgagcctgcacgccgtgggcgtgagctactggaaggccagcgagggcgccgagtacgacgaccagaccagccagcgggagaaagaagatgacaaggtgttccctggcggcagccacacctacgtgtggcaggtgctgaaagaaaacggccccatggcctccgaccccctgtgcctgacctacagctacctgagccacgtggacctggtgaaggacctgaacagcggcctgatcggcgctctgctcgtctgccgggagggcagcctggccaaagagaaaacccagaccctgcacaagttcatcctgctgttcgccgtgttcgacgagggcaagagctggcacagcgagacaaagaacagcctgatgcaggaccgggacgccgcctctgccagagcctggcccaagatgcacaccgtgaacggctacgtgaacagaagcctgcccggcctgattggctgccaccggaagagcgtgtactggcacgtgatcggcatgggcaccacacccgaggtgcacagcatctttctggaagggcacacctttctggtccggaaccaccggcaggccagcctggaaatcagccctatcaccttcctgaccgcccagacactgctgatggacctgggccagttcctgctgttttgccacatcagctctcaccagcacgacggcatggaagcctacgtgaaggtggactcttgccccgaggaaccccagctgcggatgaagaacaacgaggaagccgaggactacgacgacgacctgaccgacagcgagatggacgtggtgcggttcgacgacgacaacagccccagcttcatccagatcagaagcgtggccaagaagcaccccaagacctgggtgcactatatcgccgccgaggaagaggactgggactacgcccccctggtgctggcccccgacgacagaagctacaagagccagtacctgaacaatggcccccagcggatcggccggaagtacaagaaagtgcggttcatggcctacaccgacgagacattcaagacccgggaggccatccagcacgagagcggcatcctgggccccctgctgtacggcgaagtgggcgacacactgctgatcatcttcaagaaccaggctagccggccctacaacatctacccccacggcatcaccgacgtgcggcccctgtacagcaggcggctgcccaagggcgtgaagcacctgaaggacttccccatcctgcccggcgagatcttcaagtacaagtggaccgtgaccgtggaggacggccccaccaagagcgaccccagatgcctgacccggtactacagcagcttcgtgaacatggaacgggacctggcctccgggctgatcggacctctgctgatctgctacaaagaaagcgtggaccagcggggcaaccagatcatgagcgacaagcggaacgtgatcctgttcagcgtgttcgatgagaaccggtcctggtatctgaccgagaacatccagcggtttctgcccaaccctgccggcgtgcagctggaagatcccgagttccaggccagcaacatcatgcactccatcaatggctacgtgttcgactctctgcagctctccgtgtgtctgcacgaggtggcctactggtacatcctgagcatcggcgcccagaccgacttcctgagcgtgttcttcagcggctacaccttcaagcacaagatggtgtacgaggacaccctgaccctgttccctttcagcggcgagacagtgttcatgagcatggaaaaccccggcctgtggattctgggctgccacaacagcgacttccggaaccggggcatgaccgccctgctgaaggtgtccagctgcgacaagaacaccggcgactactacgaggacagctacgaggatatcagcgcctacctgctgtccaagaacaacgccatcgaaccccggagcttcagccagaacccccccgtgctgacgcgtagcttcagccagaacagccggcaccccagcacccggcagaagcagttcaacgccaccaccatccccgagaacgacatcgagaaaaccgacccttggtttgcccaccggacccccatgcccaagatccagaacgtgtccagcagcgacctgctgatgctgctgcggcagagccccacccctcacggcctgagcctgagcgacctgcaggaagccaagtacgagacattcagcgacgaccccagccctggcgccatcgacagcaacaacagcctgtccgagatgacccacttccggccccagctgcaccacagcggcgacatggtgttcacccccgagagcggcctgcagctgcggctgaacgagaagctgggcaccaccgccgccaccgagctgaagaagctggacttcaaggtctccagcaccagcaacaacctgatcagcaccatccccagcgacaacctggccgctggcaccgacaacaccagcagcctgggccctcccagcatgcccgtgcactacgacagccagctggacaccaccctgttcggcaagaagtccagccccctgaccgagtccggcggacccctgtccctgagcgaggaaaacaacgacagcaagctgctggaaagcggcctgatgaacagccaggaaagcagctggggcaagaatgtgtccagcacgcgtcaccagcgggagatcacccggacaaccctgcagtccgaccaggaagagatcgattacgacgacaccatcagcgtggagatgaagaaagaggatttcgatatctacgacgaggacgagaaccagagccccagaagcttccagaagaaaacccggcactacttcattgccgccgtggagaggctgtgggactacggcatgagttctagcccccacgtgctgcggaaccgggcccagagcggcagcgtgccccagttcaagaaagtggtgttccaggaattcacagacggcagcttcacccagcctctgtatagaggcgagctgaacgagcacctggggctgctggggccctacatcagggccgaagtggaggacaacatcatggtgaccttccggaatcaggccagcagaccctactccttctacagcagcctgatcagctacgaagaggaccagcggcagggcgccgaaccccggaagaacttcgtgaagcccaacgaaaccaagacctacttctggaaagtgcagcaccacatggcccccaccaaggacgagttcgactgcaaggcctgggcctacttcagcgacgtggatctggaaaaggacgtgcactctggactgattggcccactcctggtctgccacactaacaccctcaaccccgcccacggccgccaggtgaccgtgcaggaattcgccctgttcttcaccatcttcgacgagacaaagtcctggtacttcaccgagaatatggaacggaactgcagagccccctgcaacatccagatggaagatcctaccttcaaagagaactaccggttccacgccatcaacggctacatcatggacaccctgcctggcctggtgatggcccaggaccagagaatccggtggtatctgctgtccatgggcagcaacgagaatatccacagcatccacttcagcggccacgtgttcaccgtgcggaagaaagaagagtacaagatggccctgtacaacctgtaccccggcgtgttcgagacagtggagatgctgcccagcaaggccggcatctggcgggtggagtgtctgatcggcgagcacctgcacgctggcatgagcaccctgtttctggtgtacagcaacaagtgccagaccccactgggcatggcctctggccacatccgggacttccagatcaccgcctccggccagtacggccagtgggcccccaagctggccagactgcactacagcggcagcatcaacgcctggtccaccaaagagcccttcagctggatcaaggtggacctgctggcccctatgatcatccacggcattaagacccagggcgccaggcagaagttcagcagcctgtacatcagccagttcatcatcatgtacagcctggacggcaagaagtggcagacctaccggggcaacagcaccggcaccctgatggtgttcttcggcaatgtggacagcagcggcatcaagcacaacatcttcaacccccccatcattgcccggtacatccggctgcaccccacccactacagcattagatccacactgagaatggaactgatgggctgcgacctgaactcctgcagcatgcctctgggcatggaaagcaaggccatcagcgacgcccagatcacagccagcagctacttcaccaacatgttcgccacctggtccccctccaaggccaggctgcacctgcagggccggtccaacgcctggcggcctcaggtcaacaaccccaaagaatggctgcaggtggactttcagaaaaccatgaaggtgaccggcgtgaccacccagggcgtgaaaagcctgctgaccagcatgtacgtgaaagagtttctgatcagcagctctcaggatggccaccagtggaccctgttctttcagaacggcaaggtgaaagtgttccagggcaaccaggactccttcacccccgtggtgaactccctggacccccccctgctgacccgctacctgagaatccacccccagtcttgggtgcaccagatcgccctcaggatggaagtcctgggatgtgaggcccaggatctgtactgatgagcggccgctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtacgcgccactactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggtgttcctggctctgctctgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaataaattgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccctgcaattattgaagcatttatcagggttattgtctcatgagcggatacatatttgaatgtatttagaaaaataaacaaataggggttccgcgcacatttccccgaaaagtgccacctgacgtc

SEQ ID NO: 45 - DNA, 506 bp, Artificial Sequence
  Artificial polynucleotide
gcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgac

(19) United States
(12) Patent Application Publication
(10) Pub. No.: US 2019/0390221 A1
(43) Pub. Date: Dec. 26, 2019

(54) PLASMID VECTORS FOR EXPRESSION OF LARGE NUCLEIC ACID TRANSGENES

(71) Applicant: David KIEWLICH, Alameda, CA (US)

(72) Inventor: David KIEWLICH, Alameda, CA (US)

(21) Appl. No.: 16/346,847

(22) PCT Filed: Nov. 2, 2017
(86) PCT No.: PCT/US17/59786

§ 371 Date: May 1, 2019

Related U.S. Application Data
(60) Provisional application No. 62/416,617, filed on Nov. 2, 2016.

(51) Int. Cl. C12N 15/85; C12N 15/90; C12N 15/11; C12N 9/22

(52) U.S. Cl. CPC C12N 15/85; C12N 15/907; C12N 15/11; C12N 2800/107; C12N 2310/20; C12N 2800/80; C12N 2820/00; C12N 9/22

(57) ABSTRACT

Provided herein, in certain embodiments, are plasmid expression vectors and methods of use of such vectors for either transient or stable integrated expression of transgenes in eukaryotic cells. The plasmid expression vectors provided herein are less than 3.6 kb in size and can accommodate large (>5 kb) polynucleotide insertions of transgenes and homology arms for stable integration.

### CROSS-REFERENCES TO RELATED APPLICATIONS

This application claims priority to PCT application PCT/US17/59786, filed on Nov. 2, 2017, which claims priority to U.S. Provisional Application No. 62/416,617, filed Nov. 2, 2016, the disclosures of which is incorporated herein in its entireties and for all purposes.

### REFERENCE TO A “SEQUENCE LISTING,” A TABLE, OR A COMPUTER PROGRAM LISTING APPENDIX SUBMITTED ON A COMPACT DISK

The Sequence Listing written in file 888888-888001WO_ST25.TXT, created on Nov. 2, 2017, 137,811 bytes, machine format IBM-PC, MS-Windows operating system, is hereby incorporated by reference in its entirety for all purposes.

### BACKGROUND OF THE INVENTION

Existing plasmid vectors for expression of transgenes are limited in their ability to accommodate large insertions of nucleic acids. Currently, standard plasmid vectors for eukaryotic gene expression, such as pcDNA3 (InVitrogen), are relatively large in size, about 5.5 kilobases or greater. Insertion of large transgenes (>5 kb) into these vectors has a negative impact on the properties of the vector, including bacterial transformation efficiency, propagation of the vector and gene expression. The size limitation on plasmid vectors restricts their usage in gene therapy and gene replacement applications. In view of this, certain viral vector systems have been developed that can accommodate large inserts. However, viral vectors carry associated risks of viral infection and unwanted integration of viral genes into the host genome. In addition, viral vectors must still be assembled in bacteria, which limits insert size due to decreases in production efficiency. Accordingly, there is a need for suitable and safe vectors for eukaryotic expression.

### SUMMARY OF THE INVENTION

Provided herein, in certain embodiments, are plasmid expression vectors, components of the same, and methods of use of such vectors for either transient or stably integrated expression of transgenes in eukaryotic cells. The plasmid expression vectors can allow for both random and targeted integration through the insertion of homology arms at designated homology arm insertion sites. The plasmid expression vectors provided herein are less than 3.6 kb in size and can accommodate large (e.g., greater than 5 kb) polynucleotide insertions of transgenes and homology arms for stable integration.

Provided herein, in certain embodiments, are plasmid vectors comprising: (a) a prokaryotic origin of replication; (b) a eukaryotic promoter suitable for expression of one or more transgenes; (c) a multiple cloning site for insertion of the one or more transgenes; and (d) a nucleic acid encoding a selectable marker operably linked to a eukaryotic and a prokaryotic promoter, wherein the selectable marker is suitable for both prokaryotic and eukaryotic selection; wherein the vector is not greater than about or 3.6 kilobases in length.

In certain embodiments, the plasmid vector includes: (a) a prokaryotic origin of replication; (b) a eukaryotic promoter suitable for expression of one or more transgenes; (c) a multiple cloning site for insertion of the one or more transgenes; and (d) a nucleic acid encoding a selectable marker operably linked to a dual promoter including a eukaryotic promoter and prokaryotic promoter, wherein the selectable marker is suitable for both prokaryotic and eukaryotic selection; wherein the vector is not greater than 3.6 kilobases in length.

In some embodiments, the plasmid vectors are 2.8, 2.9, 3.0, 3.1, 3.2, 3.3, 3.4, 3.5, or 3.6 kilobases in length. In some embodiments, elements (a) through (d) are arranged sequentially in the 5′ to 3′ direction of the plasmid. In some embodiments, the plasmid vectors further comprise an upstream homology arm insertion site located between a prokaryotic origin of replication and the eukaryotic promoter and further comprises a downstream homology arm insertion site. In some embodiments, the downstream homology arm insertion site located after nucleic acid encoding a selectable marker but before the origin of replication. In some embodiments, the plasmid vectors further comprise a synthetic splice site between the eukaryotic promoter and the multiple cloning site that enhances stability of RNA transcribed from the eukaryotic promoter. In some embodiments, the plasmid vectors further comprise poly A sequences following the multiple cloning site. In some embodiments, the plasmid vectors further comprise an additional promotor upstream of the multiple cloning site for in vitro expression of the one or more transgenes. In some embodiments, the additional promotor for in vitro expression is a T7 promoter. In some embodiments, the origin of replication is selected from the group consisting of pBR322, pMB1, p15A, pACYC184, pACYC177, ColE1, pBR3286, p1, pBR26, pBR313, pBR327, pBR328, pPIGDM1, pPVUI, pF, pSC101 and pC101p-157. In some embodiments, the origin of replication is pBR322 Ori. In some embodiments, the eukaryotic promoter for expression of the transgene is selected from the group consisting of a cytomegalovirus (CMV) promoter, the promoter of the Beta-Actin gene from human, mouse, or chicken, the promoter of the Ubiquitin C gene, and the promoter of the Thymidine Kinase gene from Herpes Virus. In some embodiments, the eukaryotic promoter of (b) is a cytomegalovirus (CMV) promoter. In some embodiments, the selectable marker is selected from the group consisting of an antibiotic resistance gene, a fluorescent protein, and an enzyme. In some embodiments, the selectable marker is an antibiotic resistance gene. In some embodiments, the selectable marker is blasticidin S deaminase. In some embodiments, the selectable marker is a fluorescent protein. In some embodiments, the fluorescent protein is a near infrared fluorescent protein. In some embodiments, the nucleic acid encoding the selectable marker is operably linked to an SV40 promoter. In some embodiments, the nucleic acid encoding the selectable marker is operably linked to an EM7 promoter. In some embodiments, the multiple cloning site comprises the sequence set forth in nucleotides 1427 to 1479 of SEQ ID NO: 2. In some embodiments, the upstream homology arm insertion site comprises the sequence set forth in nucleotides 311 to 336 of SEQ ID NO: 2. In some embodiments, the downstream homology arm insertion site comprises the sequence set forth in nucleotides 2960 to 2985 of SEQ ID NO: 2. In some embodiments, the vector has a nucleotide sequence set forth in SEQ ID NO: 2. In some embodiments, the plasmid vectors further comprise a transgene inserted at the multiple cloning site. In some embodiments, the transgene encodes a therapeutic protein or a therapeutic RNA. In some embodiments, the length of the upstream homology arm and/or the downstream homology arm is about 500 bases to about 4 kilobases in length. In some embodiments, the transgene nucleic acid ranges from about 5 kb to 300 kb in length.

Provided herein, in certain embodiments, are methods for gene expression. In some embodiments, the methods comprise transfecting a eukaryotic cell with a plasmid vector provided herein, further comprising a transgene inserted at the multiple cloning site, and culturing the cell under conditions suitable for expression of the transgene.

Also provided herein, in certain embodiments, are methods for modifying a target genomic locus in a mammalian cell, comprising: (a) introducing into a mammalian cell: (i) a nuclease agent that makes a single or double-strand break at or near a target genomic locus, and (ii) a plasmid vector provided herein, further comprising a transgene inserted at the multiple cloning site flank an upstream homology arm inserted at the upstream homology arm insertion site and a downstream homology arm inserted at the downstream homology arm; and (b) selecting a targeted mammalian cell comprising the transgene in the target genomic locus. In some embodiments, the cell is selected by detection of the selectable marker. In some embodiments, the mammalian cell is a pluripotent cell. In some embodiments, the pluripotent cell is an induced pluripotent stem (iPS) cell, embryonic stem (ES) cell, an adult stem cell, a hematopoietic stem cell, a neuronal stem cell. In some embodiments, the mammalian cell is a human fibroblast. In some embodiments, the mammalian cell is a human embryonic kidney cell (HEK) 293. In some embodiments, the mammalian cell is a human cell isolated from a patient having a disease, and wherein the human cell comprises at least one human disease allele in its genome. In some embodiments, the mammalian cell is a Chinese Hamster Ovary (CHO) cell. In some embodiments, the mammalian cell is an immortalized African Green Monkey (COS) cell. In some embodiments, integration of the transgene into the target genomic locus replaces the at least one human disease allele in the genome. In some embodiments, the nuclease agent is an expression construct comprising a nucleic acid sequence encoding a nuclease, and wherein the nucleic acid is operably linked to a promoter active in the mammalian cell. In some embodiments, the nuclease agent is an mRNA encoding a nuclease. In some embodiments, the nuclease is a zinc finger nuclease (ZFN). In some embodiments, the nuclease is a Transcription Activator-Like Effector Nuclease (TALEN). In some embodiments, the nuclease is a meganuclease. In some embodiments, the nuclease is a Cas9 nuclease. In some embodiments, a target sequence of the nuclease agent is located in an intron, an exon, a promoter, a promoter regulatory region, or an enhancer region in the target genomic locus. In some embodiments, the target sequence is an AAV1 integration site. In some embodiments, the length of the upstream homology arm and/or the downstream homology arm for integration of the transgene is about 500 bases to about 4 kilobases. In some embodiments, the transgene nucleic acid that is integrated ranges from about 5 kb to 300 kb in length.

In some embodiments, a plasmid vector provided herein is selected from among pDK, pDK 9-1, pDK9-2, and pDK9-3_Puro, pDK9-3_Neo. In some embodiments, a plasmid vector provided herein comprises a transgene. In some embodiments, the plasmid vector comprises a factor VIII (FVIII) transgene, B-domain-deleted factor VIII (FVIII-BDD) transgene or a Phenylalanine Hydroxylase (PAH) transgene. In some embodiments, the plasmid vector is selected from among pDK9-2_FVIII-BDD and pDK9-2_PAH.

In some embodiments, the plasmid vector provided herein is a targeting vector comprising left and right homology arms for integration of nucleic acid into a genome. In some embodiments, the plasmid vector that is a targeting vector is pDK9-2_AAVS1Targeted. In some embodiments, the plasmid vector that is a targeting vector comprises a transgene. In some embodiments, the plasmid vector that is a targeting vector comprises an FVIII transgene, an FVIII-BDD transgene or a PAH transgene. In some embodiments, the plasmid vector that is a targeting vector is selected from among pDK9-2_PAH_AAVS1Targeted and pDK9-2_FVIII-BDD_AAVS1Targeted

In some embodiments, an intermediate vector for the generation of the pDK expression vectors provided herein is provided. In some embodiments, an intermediate vector is selected from among pDK7-1 and pDK8-1.

### BRIEF DESCRIPTION OF THE DRAWINGS

PM FIG. 1 illustrates a schematic diagram of a vector provided herein showing the various features of the pDK vector technology.

FIG. 2 illustrates a schematic diagram of the example vector pDK9-2.

FIG. 3 illustrates the level of transient expression of the PAH gene in 293T cells transfected with pcDNA-PAH compared to pDK-PAH. A Western blot of the cell lysates probed with anti-PAH or -GAPDH antibodies is shown.

FIG. 4 illustrates the level of stable expression of the PAH gene in 293T cells transfected with pcDNA-PAH compared to pDK-PAH and selected for stable integration. A Western blot of the cell lysates probed with anti-PAH or -GAPDH antibodies is shown.

FIG. 5 illustrates the level of transient expression of the FVIII-BDD gene in 293T cells transfected with pDK-FVIII-BDD compared to pcDNA-FVIII-BDD or empty plasmid. A Western blot of the cell lysates probed with anti-Factor VIII C-domain antibodies is shown.

FIG. 6 illustrates the number of stably integrated clones in 293 or human adipose derived stem cells (hADSC) using targeted integration at the AAV1 integration site using the Cas9 system in combination with targeting vectors pDK-PAH-AAV1, pDK-FVIII-BDD-AAV1, pcDNA-PAH-AAV1 or pcDNA-FVIII-BDD-AAV1.

FIG. 7 illustrates a schematic diagram of the starting vector pCI-neo (Promega).

FIG. 8 illustrates a schematic diagram of the intermediate vector pDK7-1.

FIG. 9 illustrates a schematic diagram of the intermediate vector pDK8-1.

FIG. 10 illustrates a schematic diagram of the intermediate vector pDK9-1

FIG. 11 illustrates a schematic diagram of the vector pDK9-2 (blasticidin).

FIG. 12 illustrates a schematic diagram of the vector pDK9-3_Puro.

FIG. 13 illustrates a schematic diagram of the vector pDK9-3_Neo.

FIG. 14 illustrates a schematic diagram of the vector pDK9-2_FVIII-BDD.

FIG. 15 illustrates a schematic diagram of the vector pcDNA6_FVIII-BDD.

FIG. 16 illustrates a schematic diagram of the vector pDK9-2_PAH.

FIG. 17 illustrates a schematic diagram of the vector pcDNA6_PAH.

FIG. 18 illustrates a schematic diagram of the vector pDK9-2_AAVS1Targeted.

FIG. 19 illustrates a schematic diagram of the vector pDK9-2_PAH_AAVS1Targeted.

FIG. 20 illustrates a schematic diagram of the vector pDK9-2_FVIIIBDD_AAVS1Targeted.

FIG. 21 illustrates a schematic diagram of the vector pcDNA6-PAH_AAVS1Targeted.

FIG. 22 illustrates a schematic diagram of the vector pcDNA6-FVIIIBDD_AAVS1Targeted.

FIG. 23 illustrates a schematic diagram of the vector pDK-Streamline (also referred to herein as pDK).

FIG. 24 illustrates a schematic diagram of the vector pDK-Streamline with the expression vector main promoter location circled.

FIG. 25 illustrates a schematic diagram of the vector pDK-Streamline with the selectable hybrid promoter location circled.

FIG. 26 illustrates a schematic diagram of the vector pDK-Streamline with the right and left homology insertion sites circled.

FIG. 27 illustrates a schematic diagram of the vector pDK-Streamline with the artificial splice site circled.

FIG. 28 illustrates a schematic diagram of the vector pDK-Streamline with the T7 promoter location circled.

FIG. 29 illustrates a schematic diagram of the vector pDK-Streamline with the two expression cassette parts of the vector circled.

FIGS. 30A-30B. FIG. 30A illustrates a schematic diagram of the vector pDK-Streamline with the expression cassette for bacterial and mammalian selection circled. FIG. 30B illustrates a schematic diagram of a commercially available vector from Invitrogen containing separate bacterial and mammalian selectable markers. The separate bacterial and mammalian selectable markers are circled. Note that the commercial vector is nearly 2000 bp larger compared to the pDK-Streamline vector.

FIG. 31 is a schematic representation of using CRISPR technology to insert (i.e., “knock-in”) a sequence obtained from a vector that included homology arms. The black rectangle in the “Before” genome represents the location of the CRISPR break site. Once CRISPR is added, a double strand break occurs at the CRISPR site. The light gray rectangle of the vector represents the sequence to be inserted into the genome, and the flanking rectangles are homologous with the regions flanking the break site in the genome. The new sequence is inserted into the genome at the site of the break. This insertion only works if the homology arms are identical to the sequence around the break site.

FIGS. 32A-32B. FIG. 32A illustrates a schematic diagram of the circular vector pDK-Streamline with arrows pointing to the homology sites. FIG. 32B is a linear representation of FIG. 32A.

FIG. 33 shows a linear representation of the pDK-Streamline vector with arrows pointing to the regions that can be targeted using enzyme blends. The blends can be used to remove or change the left arm or right arm homology domains or a blend can be used to linearize the circular vector.

FIG. 34 illustrates the vector map for pDK-Streamline1-Blast (also referred to herein as pDK9-2; SEQ ID NO:2).

FIG. 35 illustrates the vector map for pDK-Streamline1-Puro (also referred to herein as pDK9-3_Puro; SEQ ID NO:4).

FIG. 36 illustrates the vector map for pDK-Streamline1-Neo (also referred to herein as pDK9-3_Neo; SEQ ID NO:3).

### DETAILED DESCRIPTION OF THE INVENTION

Described herein are vectors, components, and kits for the expression of one or more transgenes either by transient transfection or stable integration via random or targeted recombination. As described herein, the present technology is based in part on the observation that capacity and efficacy of traditional plasmid expression vectors can be enhanced by the elimination of excess non-functional sequences. By taking a de novo approach to vector assembly, a compact plasmid expression vector was generated that incorporates elements needed for high copy replication, high efficiency gene expression, genome integration, and selection in a highly ordered and space efficient manner. The vectors can contain components for prokaryotic replication, prokaryotic and eukaryotic gene expression, for example, of a single selection marker that is functional for selection in both prokaryotes and eukaryotes, promoters for robust expression of one or more transgenes in cell and cell-free environments as well as additional elements to increase protein expression, such as synthetic RNA splice sites. Due to their smaller base pair size of less than 3.6 kb, these expression vectors have a higher capacity for larger polynucleotide insertions of transgenes or multiple transgenes and longer homology arms for stable integration. One non-limiting example of a vector provided herein is pDK9, which is represented by the nucleic acid sequence set forth in SEQ ID NO: 1. In some embodiments the vectors can have a size of less than or not greater than 3.6 kb, for example, between 1.5 and 3.6 kb, or any sub value or subrange there between, and can include the endpoints.

### I. Definitions

Unless defined otherwise, all technical and scientific terms used herein have the same meaning as commonly understood by one of ordinary skill in the art to which this disclosure belongs.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise.

As used herein, the term “about” means that a value may vary +/−20%, +/−15%, +/−10% or +/−5% and remain within the scope of the present disclosure.

The term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed subject matter. “Consisting of” shall mean excluding more than trace amount of other ingredients and substantial method steps recited. Embodiments defined by each of these transition terms are within the scope of this technology and each of the terms is contemplated for use with any of embodiments described herein.

As will be understood by one skilled in the art, for any and all purposes, particularly in terms of providing a written description, all ranges disclosed herein also encompass any and all possible subvalues, subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

In addition, where features or aspects of the disclosure are described in terms of Markush groups, those skilled in the art will recognize that the disclosure is also thereby described in terms of any individual member or subgroup of members of the Markush group.

As used herein, the terms “isolated,” “purified” or “substantially purified” refer to molecules, such as nucleic acid molecules or polypeptides, that are removed from their natural environment, isolated or separated, and are at least 60% free, preferably 75% free, and most preferably 90% free from other components with which they are naturally associated. An isolated molecule is therefore a substantially purified molecule.

The terms “identity” and “identical” refer to a degree of identity between sequences. There can be partial identity or complete identity. A partially identical sequence is one that is less than 100% identical to another sequence. Partially identical sequences can have an overall identity of at least 70% or at least 75%, at least 80% or at least 85%, or at least 90% or at least 95%.

The term “detectable label” as used herein refers to a molecule or a compound or a group of molecules or a group of compounds associated with a probe and is used to identify the probe hybridized to a nucleic acid molecule, such as a genomic nucleic acid molecule, an RNA nucleic acid molecule, a cDNA molecule or a reference nucleic acid.

As used herein, the term “detecting” refers to observing a signal from a detectable label to indicate the presence of a target. More specifically, detecting is used in the context of detecting a specific sequence of a target nucleic acid molecule. The term “detecting” used in context of detecting a signal from a detectable label to indicate the presence of a target nucleic acid in the sample does not require the method to provide 100% sensitivity and/or 100% specificity. A sensitivity of at least 50% is preferred, although sensitivities of at least 60%, at least 70%, at least 80%, at least 90%, or at least 99% are more preferred. A specificity of at least 50% is preferred, although sensitivities of at least 60%, at least 70%, at least 80%, at least 90%, or at least 99% are more preferred. Detecting also encompasses assays that produce false positives and false negatives. False negative rates can be 1%, 5%, 10%, 15%, 20% or even higher. False positive rates can be 1%, 5%, 10%, 15%, 20% or even higher.

As used herein, the terms “amplification” and “amplify” encompass all methods for copying or reproducing a target nucleic acid molecule having a specific sequence, thereby increasing the number of copies or amount of the nucleic acid sequence in a sample. The amplification can be exponential or linear. The target nucleic acid can be DNA or RNA. A target nucleic acid amplified in this manner is referred to herein as an “amplicon.” While illustrative methods described herein relate to amplification using the polymerase chain reaction (PCR), numerous other methods are known in the art for amplification of nucleic acids, such as, but not limited to, isothermal methods, rolling circle methods, etc. The skilled artisan understands that these other methods can be used either in place of, or in conjunction with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 April; 30(4):852-6, 858, 860; Zhong, et al., Biotechniques 2001 April; 30(4):852-6, 858, 860; each of which is incorporated herein by reference in its entirety.

As used herein, the term “oligonucleotide” refers to a short nucleic acid polymer composed of deoxyribonucleotides, ribonucleotides, or any combination thereof. Oligonucleotides are generally between about 10, 11, 12, 13, 14, 15, 16, 17, 18, 19, 20, 25, or 30 to about 150 nucleotides (nt) in length, more preferably about 10, 11, 12, 13, 14, 15, 16, 17, 18, 19, 20, 25, or 30 to about 70 nt in length. An oligonucleotide can be used as a primer or as a probe according to methods described herein and known generally in the art.

As used herein, an oligonucleotide that is “specific” for a nucleic acid is one that, under the appropriate hybridization or washing conditions, is capable of hybridizing to the target of interest and not substantially hybridizing to nucleic acids that are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well-known in the art.

A “primer” for nucleic acid amplification is an oligonucleotide that specifically anneals to a target nucleotide sequence and leads to addition of nucleotides to the 3′ end of the primer in the presence of a DNA or RNA polymerase. As known in the art, the 3′ nucleotide of the primer should generally be identical to the target nucleic acid sequence at a corresponding nucleotide position for optimal expression and amplification. The term “primer” as used herein includes all forms of primers that can be synthesized including, but not limited to, peptide nucleic acid primers, locked nucleic acid primers, phosphorothioate modified primers, labeled primers, and the like. Primers can be naturally occurring as in a purified from a biological sample or from a restriction digest or produced synthetically. In some embodiments, primers can be approximately 15-100 nucleotides in length, typically 15-25 nucleotides in length. The exact length of the primer will depend upon many factors, including hybridization and polymerization temperatures, source of primer and the method used. For example, for diagnostic applications, depending on the complexity of the target sequence, the oligonucleotide primer typically contains 15-25 or more nucleotides, although it may contain fewer or more nucleotides. The factors involved in determining the appropriate length of primer are readily known to one of ordinary skill in the art. One of skill in the art understands that the terms “forward primer” and “reverse primer” refer generally to primers complementary to sequences that flank the target nucleic acid and are used for amplification of the target nucleic acid. Generally, a “forward primer” is a primer that is complementary to the anti-sense strand of DNA, and a “reverse primer” is complementary to the sense-strand of DNA.

As used herein, a “probe” refers to a type of oligonucleotide having or containing a sequence which is complementary to another polynucleotide, e.g., a target polynucleotide or another oligonucleotide. The probes for use in the methods described herein are ideally less than or equal to 500 nucleotides in length, typically between about 10 nucleotides to about 100, e.g. about 15 nucleotides to about 40 nucleotides. The probes for use in the methods described herein are typically used for detection of a target nucleic acid sequence by specifically hybridizing to the target nucleic acid. Target nucleic acids include, for example, a genomic nucleic acid, an expressed nucleic acid, a reverse transcribed nucleic acid, a recombinant nucleic acid, a synthetic nucleic acid, an amplification product or an extension product as described herein.

The term “complement” “complementary” or “complementarity” with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-S′.” Certain bases not commonly found in natural nucleic acids can be included in the nucleic acids described herein; these include, for example, inosine, 7-deazaguanine, Locked Nucleic Acids (LNA), and Peptide Nucleic Acids (PNA). Complementary need not be perfect; stable duplexes can contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “administration” of an agent to a subject includes any route of introducing or delivering the agent to a subject to perform its intended function. Administration can be carried out by any suitable route, including intravenously, intramuscularly, intraperitoneally, or subcutaneously. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic peptide administered to the subject may depend on the type and severity of the infection and on the characteristics of the individual, such as general health, age, sex, body weight and tolerance to drugs. It may also depend on the degree, severity and type of disease. The skilled artisan will be able to determine appropriate dosages depending on these and other factors.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

The terms “patient,” “subject,” “individual,” and the like are used interchangeably herein, and refer to an animal, typically a mammal. In a preferred embodiment, the patient, subject, or individual is a mammal. In a particularly preferred embodiment, the patient, subject or individual is a human. In other embodiments, the animal can be a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like).

The terms “treating” or “treatment” as used herein covers the treatment of a disease in a subject, such as a human, and includes: (i) inhibiting a disease, i.e., arresting its development; (ii) relieving a disease, i.e., causing regression of the disease; (iii) slowing progression of the disease; and/or (iv) inhibiting, relieving, or slowing progression of one or more symptoms of the disease.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “therapeutic” as used herein means a treatment and/or prophylaxis. A therapeutic effect is obtained by suppression, remission, or eradication of a disease state.

### II. Plasmid Expression Vectors

The plasmid expression vectors provided herein contain nucleic acid elements required for plasmid replication, gene expression and target gene integration. These include bacterial replication origins for plasmid propagation and various promoters, including a dual promoter, for prokaryotic and/or eukaryotic gene expression of the selection marker and transgenes. Additional elements include, but are not limited to enhancers to increase stability of transcribed RNA and protein expression, including synthetic RNA splice sites and polyA sequences. The vectors provided herein can include one or more of the nucleic acid elements described herein. A non-limiting example of a vector provided herein is pDK9. A non-limiting description of examples of features of the vectors is provided herein.

In particular embodiments, provided herein are plasmid vectors comprising: (a) a prokaryotic origin of replication; (b) an upstream homology arm insertion site; (c) a eukaryotic promoter suitable for expression of one or more transgenes; (d) a multiple cloning site for insertion of the one or more transgenes; (e) nucleic acid encoding a selectable marker operably linked to a eukaryotic and a prokaryotic promoter, wherein the selectable marker is suitable for both prokaryotic and eukaryotic selection; and (f) a downstream homology arm insertion site, wherein elements (a) through (f) are arranged sequentially in the 5′ to 3′ direction of the plasmid.

In particular embodiments, provided herein are plasmid vectors comprising: (a) a prokaryotic origin of replication; (b) a upstream homology arm insertion site; (c) a eukaryotic promoter suitable for expression of one or more transgenes; (d) a multiple cloning site for insertion of the one or more transgenes; (e) a nucleic acid encoding a selectable marker operably linked to a dual promoter including a eukaryotic promoter and a prokaryotic promoter, wherein the selectable marker is suitable for both prokaryotic and eukaryotic selection; and (f) a downstream homology arm insertion site, wherein elements (a) through (f) are arranged sequentially in the 5′ to 3′ direction of the plasmid.

In particular embodiments, the vector is not greater than 3.6 kilobases in length. In some embodiments, the vector is 2.8, 2.9, 3.0, 3.1, 3.2, 3.3, 3.4, 3.5, or 3.6 kilobases in length. In some embodiments, the vector is about 2.8, about 2.9, about 3.0, about 3.1, about 3.2, about 3.3, about 3.4, about 3.5, or about 3.6 kilobases in length.

Some embodiments relate to vector nucleic acid sequences and vector nucleic acid element sequences as set forth herein. Some embodiments relate to the SEQ ID NOs:1-45. Some embodiments relate to sequences having 70-99.9% sequence identity to any of the sequences described herein, including all subranges and subvalues therein. In embodiments, sequence identity can be 70% to any of the sequences provided herein. In embodiments, sequence identity can be 75% to any of the sequences provided herein. In embodiments, sequence identity can be 80% to any of the sequences provided herein. In embodiments, sequence identity can be 85% to any of the sequences provided herein. In embodiments, sequence identity can be 90% to any of the sequences provided herein. In embodiments, sequence identity can be 91% to any of the sequences provided herein. In embodiments, sequence identity can be 92% to any of the sequences provided herein. In embodiments, sequence identity can be 93% to any of the sequences provided herein. In embodiments, sequence identity can be 94% to any of the sequences provided herein. In embodiments, sequence identity can be 95% to any of the sequences provided herein. In embodiments, sequence identity can be 96% to any of the sequences provided herein. In embodiments, sequence identity can be 97% to any of the sequences provided herein. In embodiments, sequence identity can be 98% to any of the sequences provided herein. In embodiments, sequence identity can be 99% to any of the sequences provided herein. In embodiments, sequence identity can be 99.5% to any of the sequences provided herein. In embodiments, sequence identity can be 99.9% to any of the sequences provided herein. In some embodiments, a sequence having a percentage identity to a sequence provided herein can have the same function as the natural sequence or full-length sequence.

Methods for determining sequence identity are well known in the art. Non-limiting examples for determining sequence identity include BLAST or BLAST 2.0 sequence comparison algorithms with default parameters or by manual alignment and visual inspection (see, e.g., NCBI web site ncbi.nlm.nih.gov/BLAST/ or the like).

In embodiments, the prokaryotic origin of replication is not an F1 origin. In embodiments, the plasmid vector includes exactly one selectable marker. For example, in some embodiments, the vector can include only a single selectable marker that functions in either or both of a prokaryotic or eukaryotic host.

### Prokaryotic Replication Origin

Generally, the vectors provided here contain a prokaryotic origin of replication, such as a bacterial replication origin. Non-limiting examples of replication origins for propagation of plasmids in prokaryotes, such as bacteria, are well known in the art and include for example, pBR322, pMB1, p15A, pACYC184, pACYC177, ColE1, pBR3286, p1, pBR26, pBR313, pBR327, pBR328, pPIGDM1, pPVUI, pF, pSC101 or pC101p-157. In particular embodiments, the bacterial replication origin is a high copy number origin of replication. In particular embodiments, the bacterial replication origin is the pBR322 origin of replication. In some embodiments, the origin also can act as a convenient place to linearize the vector.

### Homology Arm Insertion Sites

For targeted integration of nucleic acid into a host genome, the plasmid vector typically comprises nucleic acid segments that are homologous to the targeted region. These nucleic acid segments are referred to as homology arms and are inserted on either side of the nucleic acid to be inserted. In the non-limiting exemplified plasmid expression vectors provided herein, homology arm insertion sites are present that flank the expression cassette that contains the insertion site (i.e. multiple cloning site) for one or more transgenes. In particular embodiments, the homology arm insertion sites on located on either side of the high copy number prokaryotic origin of replication, in opposite orientation. This configuration ensures that the high copy replication origin is not integrated into the host genome during recombination, and thus minimizes undesired effects of integration.

The homology arm insertion sites comprise rare restriction sites. Use of rare restriction sites facilitates cloning into the vector. In a non-limiting example, a homology arm insertion site comprises a restriction site for Swa1, SbfI, AscI and/or PmeI. In particular examples, the upstream (or left) arm insertion site comprises Swa1 and/or SbfI restriction sites. In particular examples, the downstream (or right) arm insertion site comprises AscI and/or PmeI restriction sites. Inclusion of a blunt cutter restriction site, such as for SwaI or PmeI, permits insertion of a blunt fragment into the homology arm insertion site in the event that the sequence to be inserted contains the restriction site.

In some embodiments, the upstream and/or downstream insertion site can accommodate a homology arm that ranges from about 500 bases to about 4 kilobases in length, such as for example, from about 500 bases to about 3 kilobases in length, such as for example, from about 500 bases to about 2 kilobases in length, such as for example, from about 1 kilobase to about 2 kilobases in length.

In one embodiment, a sum total of the upstream homology arm and the downstream homology arm is at least 10 kb. In one embodiment, the upstream homology arm ranges from about 5 kb to about 100 kb. In one embodiment, the downstream homology arm ranges from about 5 kb to about 100 kb. In one embodiment, the upstream and the downstream homology arms range from about 5 kb to about 10 kb. In one embodiment, the upstream and the downstream homology arms range from about 10 kb to about 20 kb. In one embodiment, the upstream and the downstream homology arms range from about 20 kb to about 30 kb. In one embodiment, the upstream and the downstream homology arms range from about 30 kb to about 40 kb. In one embodiment, the upstream and the downstream homology arms range from about 40 kb to about 50 kb. In one embodiment, the upstream and the downstream homology arms range from about 50 kb to about 60 kb. In one embodiment, the upstream and the downstream homology arms range from about 60 kb to about 70 kb. In one embodiment, the upstream and the downstream homology arms range from about 70 kb to about 80 kb. In one embodiment, the upstream and the downstream homology arms range from about 80 kb to about 90 kb. In one embodiment, the upstream and the downstream homology arms range from about 90 kb to about 100 kb. In one embodiment, the upstream and the downstream homology arms range from about 100 kb to about 110 kb. In one embodiment, the upstream and the downstream homology arms range from about 110 kb to about 120 kb. In one embodiment, the upstream and the downstream homology arms range from about 120 kb to about 130 kb. In one embodiment, the upstream and the downstream homology arms range from about 130 kb to about 140 kb. In one embodiment, the upstream and the downstream homology arms range from about 140 kb to about 150 kb. In one embodiment, the upstream and the downstream homology arms range from about 150 kb to about 160 kb. In one embodiment, the upstream and the downstream homology arms range from about 160 kb to about 170 kb. In one embodiment, the upstream and the downstream homology arms range from about 170 kb to about 180 kb. In one embodiment, the upstream and the downstream homology arms range from about 180 kb to about 190 kb. In one embodiment, the upstream and the downstream homology arms range from about 190 kb to about 200 kb.

In one embodiment, the homology arms of the vector are derived from a BAC library, a cosmid library, or a P1 phage library. In one embodiment, the homology arms are derived from a genomic locus of the human or non-human animal. In one embodiment, the homology arms are derived from a synthetic DNA.

In some embodiments, the plasmids contain alternative site-specific recombination target sequences. Non-limiting examples of site-specific recombination target sequences include, but are not limited to, loxP, lox511, lox2272, lox66, lox71, loxM2, lox5171, FRT, FRT11, FRT71, attp, att, FRT, rox, and a combination of site-specific recombination target sequences thereof.

### Eukaryotic Promoter for Transgene Expression

The plasmid vectors provided herein contain eukaryotic promoters for expression of one of more transgenes. Numerous eukaryotic promoters for expression of transgenes are well known. The promoter is positioned in the plasmid to be operably linked to the nucleic acid encoding the transgene following insertion of the transgene into the multiple cloning site. Generally, a strong promoter is selected such that a consistent and high level of transgene expression is produced in a variety of cells and species. In alternative embodiments, where low expression transgene is desired, a weaker promoter may be employed. Non-limiting examples of eukaryotic promoters that can be employed include, but are not limited to, mammalian promoters, including viral promoters. In some embodiments, the promoter is a CMV promoter, EF1a promoter, SV40 promoter, PGK1 promoter, Ubc promoter, human beta actin promoter, CAG promoter, TRE promoter, UAS promoter, Ac5 promoter, polyhedrin promoter, RSV promoter, CaMKIIa promoter, GAL1, 10 promoter, TEF1 promoter, GDS promoter, ADH1 promoter, CaMV35S promoter, Ubi promoter, HSV TK promoter, H1 promoter, U6 promoter, fos promoter, or E2F promoter. In some embodiments, the eukaryotic promoter is a tissue specific promoter. Use of a tissue-specific promoter in the expression cassette can restrict unwanted transgene expression as well as facilitate persistent transgene expression. In particular embodiments, the promoter is a viral promoter. In particular embodiments, the promoter is a cytomegalovirus (CMV) promoter.

The promoter may be an inducible promoter. Non-limiting examples of inducible promoters are metallothionein promoters, alcA promoter (ethanol controlled), tetracycline-regulated promoters TetR and TetR* (the mutant form), promoters based on glucocorticoid receptor (GR), promoters based on estrogen receptor (ER), promoters based on ecdysone receptor, promoters based on various steroid/retinoid/thyroid receptor superfamily, promoters based on Xbal (cell stress transcription factor), and Heat-inducible promoters (Heat shock protein superfamily).

In some embodiments, the vector additionally contains a promoter for cell-free expression of the transgene. In some embodiments, the promoter is a viral promoter. In some embodiments, the promoter is a viral phage promoter. In some embodiments, the viral phage promoter is T7 or SP6 polymerase promoter. In addition, to priming cell-free transcription reactions, the T7 promoter site can serve as a priming site for sequencing the vector.

In some embodiments, the vector comprises a synthetic splice site. The synthetic splice site, also referred to herein as an artificial splice site, allows the transcribed RNA to be spliced and has been shown in the art to increase the stability of the transcribed RNA, resulting in increased protein expression. In some embodiments, the splice site is derived from a eukaryotic gene. In some embodiments, the splice site is based on a consensus donor site and a consensus acceptor site of a eukaryotic gene.

The synthetic splice site can also function to create a space for insertion of a selectable marker. For example, a bacterial selectable marker can be inserted into the synthetic splice site, and the bacterial selectable marker would be spliced out inside a eukaryotic cell. Thus, in some embodiments, the synthetic splice site includes a selectable marker. In embodiments, the selectable marker is a bacterial selectable marker.

### Selectable Marker

The plasmid vectors provided herein also contain a selectable marker that is operably linked to dual promoter, also referred to herein as a hybrid promoter, for eukaryotic expression and prokaryotic expression of the selectable marker. Non-limiting examples of eukaryotic promoters that can be employed include, but are not limited to, mammalian promoters, including viral promoters. In some embodiments, the promoter is a CMV promoter, EF1a promoter, SV40 promoter, PGK1 promoter, Ubc promoter, human beta actin promoter, CAG promoter, TRE promoter, UAS promoter, Ac5 promoter, polyhedrin promoter, RSV promoter, CaMKIIa promoter, GAL1, 10 promoter, TEF1 promoter, GDS promoter, ADH1 promoter, CaMV35S promoter, Ubi promoter, HSV TK promoter, H1 promoter, U6 promoter, fos promoter, or E2F promoter. In particular embodiments, the eukaryotic promoter for expression of the selectable marker is SV40. In some embodiments, the dual promoter is a universal promoter for eukaryotic expression and prokaryotic expression. Non-limiting examples of prokaryotic promoters that can be employed include, but are not limited to, T7, T7lac, SP6, araBAD, trp, lac, Ptac and pL. In some embodiments, the prokaryotic promoter is EM7. In some embodiments, the prokaryotic promoter is a P3 bacterial promoter.

The dual promoter may be constructed such that the DNA sequence of the eukaryotic promoter is 5′ to the DNA sequence of the prokaryotic promoter. Alternatively, the dual promoter may be constructed such that the DNA sequence of the prokaryotic promoter is 5′ to the DNA sequence of the eukaryotic promoter. Thus, in embodiments, the dual promoter includes a eukaryotic promoter positioned 5′ to a prokaryotic promoter. In other embodiments, the dual promoter includes a prokaryotic promoter positioned 5′ to a eukaryotic promoter.

In certain instances, the eukaryotic promoter DNA and the prokaryotic promoter DNA may have regions of homology. These homologous regions may be exploited to reduce the total length of the dual promoter, thereby decreasing the total size of the plasmid vector. For example, if the 3′ end of the eukaryotic promoter includes a nucleic acid sequence identical to the 5′ end the prokaryotic promoter, the 3′ end of the eukaryotic promoter may be used as the 5′ end of the prokaryotic promoter, or, alternatively, the 5′ end of the prokaryotic promoter may be used as the 3′ end of the eukaryotic promoter. In embodiments, the dual promoter includes the sequence of SEQ ID NO: 45. In embodiments, the dual promoter is the sequences of SEQ ID NO: 45.

A wide variety of selectable markers are known in the art. In particular embodiments here, the selectable marker is chosen such that it provided selection in both bacterial and eukaryotic host systems. In some embodiments, the selectable marker is an enzyme. Non-limiting examples of selectable markers include, but are not limited to, antibiotic resistance genes, such as blasticidin S deaminase (bs), hygromycin B phosphotransferase (hyg^{r}), puromycin-N-acetyltransferase (puro^{r}), neomycin phosphotransferase (neo^{f}), xanthine/guanine phosphoribosyl transferase (gpt), and herpes simplex virus thymidine kinase (HSV-k). In embodiments, the selectable marker is blasticidin S deaminase. In embodiments, the selectable marker is puromycin-N-acetyltransferase. In embodiments, the selectable marker is neomycin phosphotransferase.

An additional bacterial antibiotic resistance gene may be added to the vector, though it is not required. As described above, the bacterial antibiotic resistance gene may be inserted into the synthetic splice site. In some embodiments, the plasmid vector includes an additional selectable marker located, for example, within the synthetic splice site. Generally, the plasmids do not contain an additional specifically bacterial antibiotic resistance gene in order to minimize the amount of sequence space taken up by the resistance gene, which may impact the capacity of the vector. In other embodiments, no additional selectable markers are included that are not operably linked to a dual promoter or located within a synthetic splice site.

In some embodiments, the selectable marker comprises a fluorescent protein. Fluorescent proteins are useful for tracking expression in living cells and animals. In some embodiments the fluorescent protein selected from the group consisting of Near-infrared fluorescent protein (NirFP), mPlum, mCherry, tdTomato, mStrawberry, J-Red, DsRed, mOrange, mKO, mCitrine, Venus, YPet, yellow fluorescent protein (YFP), enhanced yellow fluorescent protein (EYFP), Emerald, green fluorescent protein (GFP), enhanced green fluorescent protein (EGFP), CyPet, cyan fluorescent protein (CFP), Cerulean, and T-Sapphire.

In some embodiments, the selectable marker is an enzyme selected from among LacZ, luciferase, and alkaline phosphatase. Additional selectable markers, including other fluorescent proteins, bioluminescent proteins and enzymes are known in the art. Nucleic acids encoding any of these proteins can be incorporated into the plasmid expression vectors provided. A combination of selectable markers, including two or more disclosed herein and/or known in the art. In some embodiments, the two or more selectable markers are encoded on same transcript, separated through the use of, for example, IRES site(s) or 2A peptide sequences in the vector. In some embodiments, the selectable marker is a fusion protein of two or more selectable markers.

### Example Transgenes for Insertion

In particular embodiments, the plasmid expression vectors provided herein are modified to comprise one or more transgenes inserted at a multiple cloning site downstream of the promoter described above for transgene expression. The multiple cloning site is a region of vector sequence which includes intentionally clustered restriction sites useful for ready insertion of one or more transgenes. In some embodiments, the two or more transgenes are separated by viral 2A self-cleaving ribosomal skipping sequences or an internal ribosomal entry site (IRES) for expression of the multicistronic nucleic acid sequence.

A transgene can be any polynucleotide endogenous or exogenous to the eukaryotic cell. In some embodiments, the transgene encodes a gene product, including a polypeptide or an RNA. In some embodiments, the transgene is associated with a disease or condition. In some embodiments, the transgene encodes a therapeutic protein or RNA useful for the treatment of a disease or condition.

In some embodiments, the transgene insertion ranges in size from about 5 kb to about 300 kb. In one embodiment, the transgene is from about 5 kb to about 200 kb. In one embodiment, the transgene is from about 5 kb to about 150 kb. In one embodiment, the transgene is from about 5 kb to about 100 kb. In one embodiment, the transgene is from about 5 kb to about 50 kb. In one embodiment, the transgene is from about 5 kb to about 10 kb. In one embodiment, the transgene insertion is from about 10 kb to about 20 kb. In one embodiment, the transgene insertion is from about 20 kb to about 30 kb. In one embodiment, the transgene insertion is from about 30 kb to about 40 kb. In one embodiment, the transgene insertion is from about 40 kb to about 50 kb. In one embodiment, the transgene insertion is from about 60 kb to about 70 kb. In one embodiment, the transgene insertion is from about 80 kb to about 90 kb. In one embodiment, the transgene insertion is from about 90 kb to about 100 kb. In one embodiment, the transgene insertion is from about 100 kb to about 110 kb. In one embodiment, the transgene insertion is from about 120 kb to about 130 kb. In one embodiment, the transgene insertion is from about 130 kb to about 140 kb. In one embodiment, the transgene insertion is from about 140 kb to about 150 kb. In one embodiment, the transgene insertion is from about 150 kb to about 160 kb. In one embodiment, the transgene insertion is from about 160 kb to about 170 kb. In one embodiment, the transgene insertion is from about 170 kb to about 180 kb. In one embodiment, the transgene insertion is from about 180 kb to about 190 kb. In one embodiment, the transgene insertion is from about 190 kb to about 200 kb. In one embodiment, the transgene insertion is from about 200 kb to about 210 kb. In one embodiment, the transgene insertion is from about 220 kb to about 230 kb. In one embodiment, the transgene insertion is from about 230 kb to about 240 kb. In one embodiment, the transgene insertion is from about 240 kb to about 250 kb. In one embodiment, the transgene insertion is from about 250 kb to about 260 kb. In one embodiment, the transgene insertion is from about 260 kb to about 270 kb. In one embodiment, the transgene insertion is from about 270 kb to about 280 kb. In one embodiment, the transgene insertion is from about 280 kb to about 290 kb. In one embodiment, the transgene insertion is from about 290 kb to about 300 kb.

Non-limiting examples of transgenes that can be expressed using the vectors provided herein include antibodies, growth factors, transcription factors, hormone, immunomodulatory molecules, anti-cancer genes, cytokines, chemokine, costimulatory molecules, protein ligands, tumor suppressors, toxins, and cytostatic proteins. In particular embodiments, the transgene is FVIII, FVIII-BDD or PAH. In particular embodiments, the transgene encodes heavy and light chains of an antibody separated with a 2a peptide. Non-limiting transgenes for insertion into the vector provided herein can be found, for example, in U.S. Pat. No. 8,945,839, International PCT application Pub. Nos. WO2013/163394, WO2013/0163394 and U.S. Patent Application Nos. 20120192298A1 and US20070042462, which are herein incorporated by reference in their entirety.

In some embodiments, the transgene encodes multiple genes for the treatment of a disease or condition, wherein each gene is separated with 2A peptides. In example embodiments, the transgene encodes multiple genes for the induction of pluripotent stem cells (iPS). For example, in some embodiments, the transgene encodes one or more of Oct4, Sox2, cMyc, and/or Klf4.

In one embodiment, the transgene comprises a genomic nucleic acid sequence that encodes a human immunoglobulin heavy chain variable region amino acid sequence. In one embodiment, the genomic nucleic acid sequence comprises an unrearranged human immunoglobulin heavy chain variable region nucleic acid sequence operably linked to an immunoglobulin heavy chain constant region nucleic acid sequence. In one embodiment, the immunoglobulin heavy chain constant region nucleic acid sequence is a mouse immunoglobulin heavy chain constant region nucleic acid sequence or human immunoglobulin heavy chain constant region nucleic acid sequence, or a combination thereof. In one embodiment, the immunoglobulin heavy chain constant region nucleic acid sequence is selected from a C_{H}1, a hinge, a C_{H}2, a C_{H}3, and a combination thereof. In one embodiment, the heavy chain constant region nucleic acid sequence comprises a C_{H}1-hinge-C_{H}2-C_{H}3. In one embodiment, the genomic nucleic acid sequence comprises a rearranged human immunoglobulin heavy chain variable region nucleic acid sequence operably linked to an immunoglobulin heavy chain constant region nucleic acid sequence. In one embodiment, the immunoglobulin heavy chain constant region nucleic acid sequence is a mouse immunoglobulin heavy chain constant region nucleic acid sequence or a human immunoglobulin heavy chain constant region nucleic acid sequence, or a combination thereof. In one embodiment, the immunoglobulin heavy chain constant region nucleic acid sequence is selected from a C_{H}1, a hinge, a C_{H}2, a C_{H}3, and a combination thereof. In one embodiment, the heavy chain constant region nucleic acid sequence comprises a C_{H}1-hinge-C_{H}2-C_{H}3.

In one embodiment, the transgene comprises a genomic nucleic acid sequence that encodes a human immunoglobulin light chain variable region amino acid sequence. In one embodiment, the genomic nucleic acid sequence comprises an unrearranged human λ, and/or κ light chain variable region nucleic acid sequence. In one embodiment, the genomic nucleic acid sequence comprises a rearranged human λ, and/or light chain variable region nucleic acid sequence. In one embodiment, the unrearranged or rearranged λ, and/or κ light chain variable region nucleic acid sequence is operably linked to a mouse, rat, or human immunoglobulin light chain constant region nucleic acid sequence selected from a λ, light chain constant region nucleic acid sequence and a κ light chain constant region nucleic acid sequence.

In one embodiment, the transgene comprises a human nucleic acid sequence. In one embodiment, the human nucleic acid sequence encodes an extracellular protein. In one embodiment, the human nucleic acid sequence encodes a ligand for a receptor. In one embodiment, the ligand is a cytokine. In one embodiment, the cytokine is a chemokine selected from CCL, CXCL, CX3CL, and XCL. In one embodiment, the cytokine is a tumor necrosis factor (TNF). In one embodiment, the cytokine is an interleukin (IL). In one embodiment, the interleukin is selected from IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In one embodiment, the interleukin is IL-2. In one embodiment, the human genomic nucleic acid sequence encodes a cytoplasmic protein. In one embodiment, the human genomic nucleic acid sequence encodes a membrane protein. In one embodiment, the membrane protein is a receptor. In one embodiment, the receptor is a cytokine receptor. In one embodiment, the cytokine receptor is an interleukin receptor. In one embodiment, the interleukin receptor is an interleukin 2 receptor alpha. In one embodiment, the interleukin receptor is an interleukin 2 receptor beta. In one embodiment, the interleukin receptor is an interleukin 2 receptor gamma. In one embodiment, the human genomic nucleic acid sequence encodes a nuclear protein. In one embodiment, the nuclear protein is a nuclear receptor.

In one embodiment, the transgene comprises a genetic modification in a coding sequence. In one embodiment, the genetic modification comprises a deletion mutation of a coding sequence. In one embodiment, the genetic modification comprises a fusion of two endogenous coding sequences.

In one embodiment, the transgene comprises a human nucleic acid sequence encoding a mutant human protein. In one embodiment, the mutant human protein is characterized by an altered binding characteristic, altered localization, altered expression, and/or altered expression pattern. In one embodiment, the human nucleic acid sequence comprises at least one human disease allele. In one embodiment, the human disease allele is an allele of a neurological disease. In one embodiment, the human disease allele is an allele of a cardiovascular disease. In one embodiment, the human disease allele is an allele of a kidney disease. In one embodiment, the human disease allele is an allele of a muscle disease. In one embodiment, the human disease allele is an allele of a blood disease. In one embodiment, the human disease allele is an allele of a cancer-causing gene. In one embodiment, the human disease allele is an allele of an immune system disease. In one embodiment, the human disease allele is a dominant allele. In one embodiment, the human disease allele is a recessive allele. In one embodiment, the human disease allele comprises a single nucleotide polymorphism (SNP) allele.

In one embodiment, the transgene comprises a regulatory sequence. In one embodiment, the regulatory sequence is a promoter sequence. In one embodiment, the regulatory sequence is an enhancer sequence. In one embodiment, the regulatory sequence is a transcriptional repressor-binding sequence. In one embodiment, the insert nucleic acid comprises a human nucleic acid sequence, wherein the human nucleic acid sequence comprises a deletion of a non-protein-coding sequence, but does not comprise a deletion of a protein-coding sequence. In one embodiment, the deletion of the non-protein-coding sequence comprises a deletion of a regulatory sequence. In one embodiment, the deletion of the regulatory element comprises a deletion of a promoter sequence. In one embodiment, the deletion of the regulatory element comprises a deletion of an enhancer sequence.

### Use in Prokaryotic Cells

In some embodiments, the vector can be utilized for protein expression in bacterial cells. Some embodiments relate to the use of the vectors and/or vector elements described herein in prokaryotic cells. For example, in some embodiments the vectors and/or components can be used to transfect prokaryotic cells, including to produce an amino acid sequence of interest in such cells. The vectors have the features as described herein, including for example, the relatively small kb sizes can permit the vectors and/or components to be used with recombinant nucleic acid sequences to produce amino acid sequences in prokaryotic cells. Any suitable prokaryotic cell can be used. Non-limiting examples of such prokaryotes include bacteria such as cocci, bacilli, spirochaete and vibrio. Non-limiting examples of bacteria that can be used include Escherichia coli, Pseudomonas, Corynebacteriaum, lactic acid bacteria, Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongate, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri.

### III. Methods for Homologous Recombination

In some, embodiments, the plasmid expression vector provided herein are employed as targeting vectors for homologous recombination. In some embodiments, a DNA binding protein, such as a sequence specific nuclease, is used to create a double stranded break in a target nucleic acid sequence. One or more or a plurality of double stranded breaks can be made in the target nucleic acid sequence. In one embodiment, a first nucleic acid sequence is removed from the target nucleic acid sequence and an exogenous nucleic acid sequence (i.e. transgene or expression cassette containing a transgene) is inserted into the target nucleic acid sequence between the cut sites or cut ends of the target nucleic acid sequence. According to certain aspects, a double stranded break at each homology arm increases or improves efficiency of nucleic acid sequence insertion or replacement, such as by homologous recombination. According to certain aspects, multiple double stranded breaks or cut sites improve efficiency of incorporation of a nucleic acid sequence from a targeting vector.

In example embodiments, a vector provided herein is introduced into a eukaryotic cell along with a nucleic acid sequence encoding a nuclease agent that makes a single- or double-stranded break at or near the target locus. In some embodiments, the vector comprises homology arms directed to the target locus within the genome of the eukaryotic cell. In some embodiments, the homology arms are derived from a genomic locus of a human, a non-human animal, a plant, or a fungus. In some embodiments, the homology arms of the targeting vector are derived from a BAC library, a cosmid library, or a P1 phage library. In one embodiment, the homology arms are derived from a synthetic DNA. In some embodiments, the homology arms are generated by nucleic acid amplification (e.g. PCR) of the homology arms from a target source, oligonucleotide synthesis assembly, or de novo nucleic acid synthesis.

In some embodiments, the eukaryotic cells are mammalian cells. In some embodiments the eukaryotic cells are primary cells. In some embodiments the eukaryotic cells are cell lines. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rath, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bc1-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

In one embodiment, the eukaryotic cell is a pluripotent cell. In one embodiment, the pluripotent cell is an embryonic stem (ES) cell. In one embodiment, the pluripotent cell is a non-human ES cell. In one embodiment, the pluripotent cell is an induced pluripotent stem (iPS) cell. In one embodiment, the induced pluripotent (iPS) cell is derived from a fibroblast. In one embodiment, the induced pluripotent (iPS) cell is derived from a human fibroblast. In one embodiment, the pluripotent cell is a hematopoietic stem cell (HSC). In one embodiment, the pluripotent cell is a neuronal stem cell (NSC). In one embodiment, the pluripotent cell is an epiblast stem cell. In one embodiment, the pluripotent cell is a developmentally restricted progenitor cell. In one embodiment, the pluripotent cell is a rodent pluripotent cell. In one embodiment, the rodent pluripotent cell is a rat pluripotent cell. In one embodiment, the rat pluripotent cell is a rat ES cell. In one embodiment, the rodent pluripotent cell is a mouse pluripotent cell. In one embodiment, the pluripotent cell is a mouse embryonic stem (ES) cell.

In one embodiment, the eukaryotic cell is an immortalized mouse or rat cell. In one embodiment, the eukaryotic cell is an immortalized human cell. In one embodiment, the eukaryotic cell is a human fibroblast. In one embodiment, the eukaryotic cell is a cancer cell. In one embodiment, the eukaryotic cell is a human cancer cell.

It should be understand that in some embodiments the vectors and components described herein can be used to produce amino acid sequences in non-mammalian eukaryotes. Examples of such eukaryotes include, but are not limited to, yeast such as Saccharomyces (e.g., Saccharomyces cerevisiae) and Pichia (e.g., Pichia pastoris), fungi such as Aspergillus, Trichoderma, and Myceliophthora (e.g., M. thermophila), insect cells such as those infected with viruses (e.g., baculovirus infected cells such as Sf9, Sf21 and High Five strains), and the like.

The vectors provided herein can be introduced into a cell by any suitable method know in the art for introduction of nucleic acids into cells. Examples of methods include, but are not limited to, transfection, transductions, viral transduction, microinjection, lipofection, nucleofection, nanoparticle bombardments, transformation, electroporation, or conjugation.

In some embodiments, the nuclease agent is introduced into the eukaryotic cells together with the targeting vector provided herein. In one embodiment, the nuclease agent is introduced separately from the targeting vector over a period of time. In one embodiment, the nuclease agent is introduced prior to the introduction of the targeting vector. In one embodiment, the nuclease agent is introduced following introduction of the targeting vector.

In some embodiments, combined use of the targeting vector with the nuclease agent results in an increased targeting efficiency compared to use of the targeting vector alone. In one embodiment, when the targeting vector is used in conjunction with the nuclease agent, targeting efficiency of the targeting vector is increased at least by two-fold compared to when the targeting vector is used alone. In one embodiment, when the targeting vector is used in conjunction with the nuclease agent, targeting efficiency of the targeting vector is increased at least by three-fold compared to when the targeting vector is used alone. In one embodiment, when the targeting vector is used in conjunction with the nuclease agent, targeting efficiency of the targeting vector is increased at least by four-fold compared to when the targeting vector is used alone.

In one embodiment, the nuclease agent is an expression construct comprising a nucleic acid sequence encoding a nuclease, wherein the nucleic acid sequence is operably linked to a promoter. In one embodiment, the promoter is a constitutively active promoter. In one embodiment, the promoter is an inducible promoter. In one embodiment, the nuclease agent is an mRNA encoding an endonuclease.

In some embodiments, the nuclease agent is a zinc-finger nuclease (ZFN). In one embodiment, each monomer of the ZFN comprises 3 or more zinc finger-based DNA binding domains, wherein each zinc finger-based DNA binding domain binds to a 3 bp subsite. In one embodiment, the ZFN is a chimeric protein comprising a zinc finger-based DNA binding domain operably linked to an independent nuclease. In one embodiment, the independent endonuclease is a Fokl endonuclease. In one embodiment, the nuclease agent comprises a first ZFN and a second ZFN, wherein each of the first ZFN and the second ZFN is operably linked to a Fokl nuclease, wherein the first and the second ZFN recognize two contiguous target DNA sequences in each strand of the target DNA sequence separated by about 6 bp to about 40 bp cleavage site, and wherein the Fokl nucleases dimerize and make a double strand break.

In some embodiments, the nuclease agent is a Transcription Activator-Like Effector Nuclease (TALEN). In one embodiment, each monomer of the TALEN comprises 12-25 TAL repeats, wherein each TAL repeat binds a 1 bp subsite. In one embodiment, the nuclease agent is a chimeric protein comprising a TAL repeat-based DNA binding domain operably linked to an independent nuclease. In one embodiment, the independent nuclease is a Fokl endonuclease. In one embodiment, the nuclease agent comprises a first TAL-repeat-based DNA binding domain and a second TAL-repeat-based DNA binding domain, wherein each of the first and the second TAL-repeat-based DNA binding domain is operably linked to a Fokl nuclease, wherein the first and the second TAL-repeat-based DNA binding domain recognize two contiguous target DNA sequences in each strand of the target DNA sequence separated by about 6 bp to about 40 bp cleavage site, and wherein the Fokl nucleases dimerize and make a double strand break at a target sequence

In some embodiments, the targeting vectors provided herein are used in combination with a Type II CRISPR system to generate single and/or double strand breaks in the host genome. In particular embodiments, a nuclease, such as the Cas9 nuclease, is guided to a target site by a guide RNA. The guide RNA and the nuclease form a co-localization complex at the DNA, upon which the nuclease induces breaks in the target DNA. In the example embodiments, where the nuclease is Cas9, the Cas9 generates a blunt-ended double-stranded break 3 bp upstream of a protospacer-adjacent motif (PAM) in the target genome via a process mediated by two catalytic domains in the protein.

Non-limiting examples of CRISPR enzymes include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the CRISPR enzyme is a Cas9 enzyme. In some embodiments, the Cas9 enzyme is S. pneumoniae, S. pyogenes or S. thermophilus Cas9, or mutants derived thereof in these organisms. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity.

Non-limiting examples of methods for homology recombination and gene editing using various nuclease systems can be found, for example, in U.S. Pat. No. 8,945,839, International PCT application Pub. No. WO2013/163394 and U.S. Patent Application Nos. 2016/0060657, 20120192298A1 and US20070042462, each of which are herein incorporated by reference in their entirety. These and any other known methods for homologous recombination can be used with the plasmid vectors provided herein.

### Therapeutic Applications

The expression vectors provided herein can be employed for expression of transgene encodes a therapeutic protein or RNA useful for the treatment of a disease or condition. In some embodiments, the vectors are employed for gene repair (e.g. gene replacement) in a subject having a genomic disease, (e.g. Hemophilia A, Phenylketonuria (PKU), sickle cell anemia, and Beta-Thalassemia, Stargardt disease, Duchenne muscular dystrophy, cystic fibrosis, Usher disease), or gene alteration for cancer suppression, HIV resistance, graft rejection, and autoimmunity. In some embodiments, the vectors are employed for the expression of therapeutic protein in a subject for the treatment of a disease or condition. For example, an expression cassette for a therapeutic protein, such as an antibody (e.g. Herceptin), a factor Xa inhibitor (e.g. an anticoagulant), or a growth factor for enhanced healing (BGF for osteoporosis). In some embodiments, the vectors can be employed for the expression of a therapeutic protein construct in a subject (e.g. a VEGF trap, a soluble receptor fusion protein, which comprises the extramembrane fragments of receptors 1 and 2 of VEGF fused to IgG1 FC fragment for treatment of wet AMD, or antibody fragments/constructs (such as single chain antibodies) for the treatment of cancer or autoimmunity). Non-limiting examples of diseases and conditions treatable with by genetic replacement and/or expression of therapeutic proteins and their associated genes are provided in U.S. Pat. No. 8,945,839, International PCT application Pub. No. WO2013/163394 and U.S. Patent Application Nos. 20120192298A1 and US20070042462, each of which are herein incorporated by reference in their entirety. In particular embodiments, plasmid vectors provided herein comprising an FVIII or FVIII-BDD transgene can be employed to treat Hemophilia A, plasmid vectors provided herein comprising a phenylalanine hydroxylase (PAH) transgene can be employed to treat phenylketonuria (PKU), plasmid vectors provided herein comprising an ABC4 transgene can be employed to treat Stargardt Disease, plasmid vectors provided herein comprising a minidystrophin transgene can be employed to treat Duchenne Muscular Dystrophy, plasmid vectors provided herein comprising a cystic fibrosis transmembrane receptor (CFTR) transgene can be employed to treat cystic fibrosis, plasmid vectors provided herein comprising an ABC4 transgene can be employed to treat Stargardt Disease.

The vectors provided herein can be administered to a subject via any suitable method of administering nucleic acids.

### Kits

The vectors or vector components provided herein may be included in a kit. In some embodiments, the kit is contemplated as being useful for manipulating the components of the vector (e.g., changing homology arms, linearizing the vector), amplifying the vector, and/or facilitating homologous recombination. The kits can include, for example, one or more of the various components of the vectors as described herein. The components can be provided together or individually with instructions for their incorporation and use. Non-limiting examples of the components include origins of replication, promoters, restriction sites, poly A sequences, selection promoters (including hybrid promoters as described herein), selectable markers (including markers that work in both eukaryotic and prokaryotic organisms), homology insertion sites, components for the promotion of integration or homologous recombination (e.g., CRISPR components and materials or others as described herein), RNA stabilizing splice sites, T7 promoters or other promoters for cell free expression, and the like. Additional kit components, can include without limitation, growth medium as described herein (e.g., agar plates), with and without a selection material (e.g., antibiotic), antibiotics, prokaryotic and eukaryotic cultures (e.g., bacterial cultures, yeast cultures and mammalian cell cultures), and the like. In some aspects, any one or more of the components described above and elsewhere herein can be specifically excluded from the kits or vectors. In some aspects, for example, the kits and vectors can specifically exclude one or more of more than one selection markers (e.g., more than one antibiotic selection marker or more than one antibiotic, more than one antibiotic plate or growth media), F1 origin of replication, an SV40 origin of replication, etc.

In some embodiments is provided a kit including the vector or components as provided herein, including embodiments thereof, and a growth medium including an antibiotic or other type of selection marker.

The growth medium provided in the kit is useful for growing cells (i.e., prokaryotic or eukaryotic cells) and further aids in determining which cells successfully took up the vector through inclusion of an antibiotic or other selection marker. The growth medium as provided herein, including embodiments thereof, can be used with eukaryotic cells. The growth medium as provided herein, including embodiments thereof, can be used with prokaryotic cells.

In embodiments, the growth medium is a liquid growth medium, a solid growth medium, or a semi-solid growth medium. In embodiments, the growth medium is agar. The kit may include pre-made agar plates or a liquid growth medium including antibiotics. In embodiments, the antibiotic included in the growth medium is blasticidin S, puromycin, or neomycin. The antibiotic can be one that limits or reduces the growth of both eukaryotic and prokaryotic cells.

Due to the fact that prokaryotic cells, such as bacteria, are naturally more resistant to certain antibiotics, the concentration of the antibiotics in the prokaryotic growth medium provided in the kit may be higher than that commonly used (e.g. 5 μg/ml of puromycin, or 10-20 μg/ml of blasticidin S) for selection of eukaryotic cells to ensure that the bacterial hosts will be limited or killed if the cell has not successfully taken up the vector. In embodiments, the concentration of antibiotic can be between at least 5 μg/ml and 150 μg/ml, or any sub value or subrange there between. For example, the amount can be at least 50 μg/ml. In embodiments, the concentration of antibiotic is 50 μg/ml. In embodiments, the concentration of antibiotic is at least 60 μg/ml. In embodiments, the concentration of antibiotic is 60 μg/ml. In embodiments, the concentration of antibiotic is at least 70 μg/ml. In embodiments, the concentration of antibiotic is 70 μg/ml. In embodiments, the concentration of antibiotic is at least 80 μg/ml. In embodiments, the concentration of antibiotic is 80 μg/ml. In embodiments, the concentration of antibiotic is at least 90 μg/ml. In embodiments, the concentration of antibiotic is 90 μg/ml. In embodiments, the concentration of antibiotic is at least 100 μg/ml. In embodiments, the concentration of antibiotic is 100 μg/ml.

The kit may also include restriction enzymes to facilitate removal of the origin of replication, thereby linearizing the vector, or removal of the homology arms, for example, for replacement. The restriction enzymes may be provided as a blend of restriction enzymes that target the restriction site on either side of the left homology arm, right homology arm, or the restriction sites flanking the origin of replication. Thus, in embodiments, the kit includes a fist, a second, and a third blend of restriction enzymes. In embodiments, the first blend of restriction enzymes can include, for example, restriction enzymes for restriction sites SwaI and SbfI; the second blend of restriction enzymes may include, for example, restriction enzymes for restriction sites AscI and PmeI; and the third blend of restriction enzymes may include, for example, restriction enzymes for restriction sites PmeI and SwaI.

The kits, as mentioned above, may also include parts useful for promoting homologous recombination of the vector into a genomic location of interest. CRISPR, TALEN, and zinc-finger nuclease genome editing systems are useful tools for generating double-strand breaks at specific genomic regions of interest (e.g., exons, introns, genes associated with diseases or disorders).

CRISPR systems (e.g., Type II systems) typically include a guide RNA (gRNA) designed to associate with a CRISPR-associated endonuclease (e.g., Cas9) and which includes a target nucleotide sequence that targets (e.g., binds) the genomic sequence to be modified and a CRISPR-associated endonuclease (e.g., Cas9) that makes the DNA double-strand break. In embodiments, the kit further includes a Type II CRISPR system for genome editing.

TALEN systems typically include transcription activator-like (TAL) effectors of plant pathogenic Xanothomonas spp fused to a Fokl nuclease. Genomic targeting specificity is accomplished through customization of the polymorphic amino acid repeats in the TAL effectors. In embodiments, the kit further includes a TALEN system for genome editing.

Zinc-finger nuclease systems typically include a zinc-finger nuclease including two functional domains. The first domain is a DNA binding domain including two-finger modules, each of which recognize a unique sequence of DNA, and are fused to create a zinc-finger protein. The second domain is a DNA-cleaving domain that includes the nuclease domain of Fokl. The first and second domains are fused, thereby creating a complex that cleaves double-stranded DNA at a target genomic location defined by the zinc-finger protein. In embodiments, the kit further includes a zinc-finger nuclease system for genome editing.

As already noted above, any one or more of the kit parts and components as described herein can be included or specifically excluded from the various embodiments.

### EXAMPLES

### Example 1. Generation of the pDK9 Vector

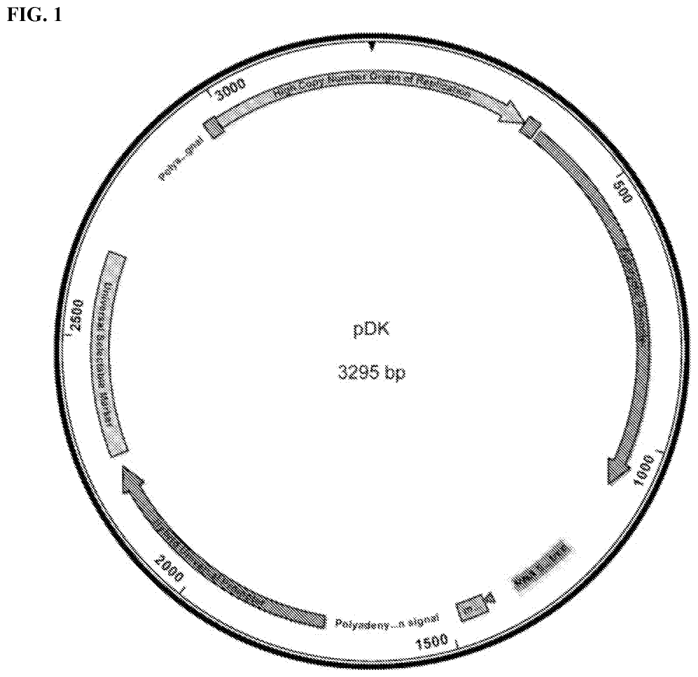

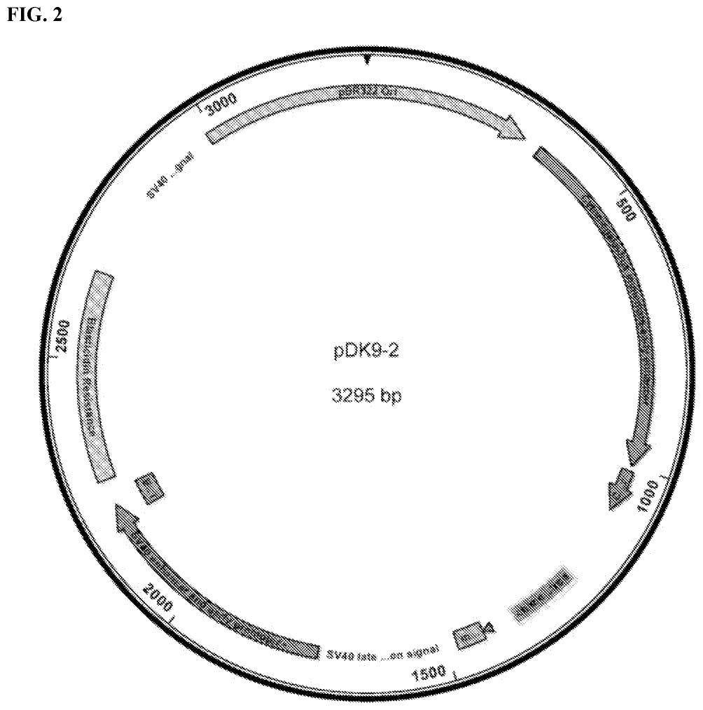

In this example, a description of the methods employed for generation of the example vector pDK9 is provided. A schematic diagram of the pDK9 vector is provided in FIG. 2. The final size of the pDK9 vector is 3.3 kb. Non-limiting examples of nucleic acid sequences of pDK9 vectors are provided as SEQ ID NOS: 1 (pDK9-1), 2 (pDK9-2), 3 (pDK9-3_Neo), and 4 (pDK9-3_Puro). Construction of each of these vectors is described herein below.

Removal of F1 origin

The phage F1 replication origin in the pCI-Neo vector (Promega; SEQ ID NO: 5) was removed PCR and excision ligation. A first PCR was performed to amplify a 257 base pair product on one side of the origin and comprises the Not 1 restriction site of the multiple cloning site and the polyA site, and introduces a DraIII restriction site via the reverse oligo after the polyA site. The PCR product was amplified with the following primers:

Forward primer:
(SEQ ID NO: 6)
5′GACCCGGGCGGCCGCTTCCCTTTAGTGAGGGTTAA3′
Reverse primer:
(SEQ ID NO: 7)
5′TGCTGCCACTCCGTGTACCACATTTGTAGAGGTTTTACTTGC3′

A second PCR was performed to amplify a 396 base pair product on the other side of the origin and comprises and SV40 promoter. A DraIII restriction site was introduced before the SV40 promoter via the forward oligo. The product also comprises the AvrII restriction site which is present at the end of the SV40 promoter. The PCR product was amplified with the following primers:

Forward primer:
(SEQ ID NO: 8)
5′GTGGTACACGGAGTGGCAGCACCATGGCCTGAAATAACCTCT3′
Reverse primer:
(SEQ ID NO: 9)
5′ CAAAAGCCTAGGCCTCCAAAAAAGCCTCCTCAC 3′

The pCI-Neo was digested with Not1 and AvrII, the PCR1 product was digested with NotI and DraIII, and the PCR2 product was digested with DraIII and AvrII. A 3-way ligation was then performed to ligate the PCR products into the cut vector. The resulting vector has the PhageF1 Origin removed and is called pDK7-1 (SEQ ID NO: 10).

### Introduction of Blasticidin Resistance Gene

The pcDNA6 vector which contains the Blasticidin resistance gene was digested with Xma1, blunted and religated to destroy Xma1 site.

A first PCR was performed to amplify from resulting vector a product comprising an AvrII site including the EM7 Promoter in primer. The PCR product was amplified with the following primers:

Forward primer:
(SEQ ID NO: 11)
5′GGAGGCCTAGGCTTTTGCAAAAAGCTGAGC3′
Reverse primer:
(SEQ ID NO: 12)
5′TCGTATTATACTATGCCGATATACTATGCCGATGATTAATTGTCAACA
CGTGCTG3′

A second PCR was performed to amplify from the overlap in the EM7 promoter in oligo through the Blasticidin resistance gene to the BstZ17I restriction site in the vector. The PCR product was amplified with the following primers:

Forward primer:
(SEQ ID NO: 13)
5′CAGCACGTGTTGACAATTAATCATCGGCATAGTATATCGGCATAGTAT
AATACGA 3′
Reverse primer:
(SEQ ID NO: 14)
5′ TCGACGGTATACAGACATGATAAGATACATTGATGAG 3′

The two PCR products were ligated together and extended to produce the EM7 Blasticidin insert.

The pDK7-1 was digested with AvrII and BsrBI, which removes the Neomycin resistance gene. The EM7 Blasticidin resistance insert was digested with AvrII and BstZ17I. The Blasticidin resistance insert was then ligated into the cut pDK7-1 vector, generating vector pDK8-1 (SEQ ID NO: 15). BstZ17I and BsrBI are blunt cutters, thus, ligating them together destroys both sites.

pDK8-1 was then digested with BspHI and re-ligated to generate pDK9-1 (SEQ ID NO: 1).

### Adding 8 Base Cutters for the Homology Arms

A PCR was performed to amplify from BspHI site to BgIII site, comprising the pBR322 origin of replication, in pDK9-1. AscI and PmeI restriction sites were introduced in the forward oligo primer. SwaI and SbfI restriction sites were introduced in the reverse oligo primer.

Forward primer:
(SEQ ID NO: 16)
5′TGAGTTTCATGAGGCGCGCCCGTCAGACCCGTTTAAACAGATCAAAGG
ATCTTCTTGAGA3′
Reverse primer:
(SEQ ID NO: 17)
5′TATTGAAGATCTCCTGCAGGCAGGAACCGTATTTAAATCGCGTTGCTG
GCGTTTTTCCAT3′

The pDK9-1 vector and the PCR product were digested with BspHI and BgIII and ligated to generate vector pDK9-2 (SEQ ID NO: 2).

Introduction of Puromycin Resistance Gene (alternative to Blasticidin resistance gene)

As an alternative to the blastocidin resistance gene, a puromycin resistance gene was cloned into the vector

PCR was used to assemble a puromycin resistance cassette:

A first PCR (PCR1) was performed to amplify AvrII through SV40 Promoter/EM7 promoter and including an overlap with a second PCR (PCR2), using the following primers:

Forward primer:
(SEQ ID NO: 18)
5′TTTGGAGGCCTAGGCTTTTGCAAAAAGCTCC3′
Reverse primer:
(SEQ ID NO: 19)
5′GAGGCGCACCGTGGGCTTGTACTCGGTCATGGTGGCGTTTAGTTCCTC
ACCTTGTCG3′

A second PCR (PCR2) was performed to amplify from a PCR1 product overlap to Puromycin resistance to the Nae1 site, using the following primers:

Forward primer:
(SEQ ID NO: 20)
5′CGACAAGGTGAGGAACTAAACGCCACCATGACCGAGTACAAGCCCACG
GTGCGCCTC3′
Reverse primer:
(SEQ ID NO: 21)
5′CATCCAGCCGGCTCAGGCACCGGGCTTGCGGGTC3′

The PCR1 and PCR2 products were mixed and extended at the two ends by PCR to generate PCR product 3.

The pDK9-2 vector and the product of PCR3 were digested with AvrII and NaeI and ligate to generate vector pDK9-3Puro (SEQ ID NO: 3).

### Introduction of Neomycin Resistance (Alternative to Blasticidin Resistance Gene)

As an alternative to the blastocidin resistance gene, a neomycin resistance gene was cloned into the vector.

Use PCR to assemble Neomycin resistance cassette:

A first PCR (PCR1) was performed to amplify AvrII through SV40 Promoter/EM7 promoter and including an overlap with a second PCR (PCR2), using the following primers:

Forward primer:
(SEQ ID NO: 22)
5′ TTTGGAGGCCTAGGCTTTTGCAAAAAGCTCC 3′
Reverse primer:
(SEQ ID NO: 23)
5′GTGCAATCCATCTTGTTCAATCATGGTGGCGTTCCTCACCTTGTCGTA
TTATACTATGC3′

A second PCR (PCR2) was performed to amplify from a PCR1 product overlap to Neomycin resistance to the Nae1 site, using the following primers:

Forward primer:
(SEQ ID NO: 24)
5′GCATAGTATAATACGACAAGGTGAGGAACGCCACCATGATTGAACAAG
ATGGATTGCAC3′
Reverse primer:
(SEQ ID NO: 25)
5′ CATCCAGCCGGCTCAGGCACCGGGCTTGCGGGTC 3′.

The PCR1 and PCR2 products were mixed and extended at the two ends by PCR to generate PCR product 3.

The pDK9-2 vector and the product of PCR3 were digested with AvrII and NaeI and ligate to generate vector pDK9-3Neo (SEQ ID NO: 4).

### Example 2. Generation and Characterization of the pDK-PAH Vector

In this example, the ability of the pDK vector to function as an expression vector was assessed by generating a pDK9 vector comprising a test nucleic acid encoding the cytosolic protein phenylalanine hydroxylase (PAH) (˜1 kb). A description of the methods for the cloning of the nucleic acid encoding PAH into the pDK9-2 vector is provided.

### Vector Construction

To make the Phenylalanine Hydroxylase (PAH) expression vector, the PAH gene was PCR amplified from a commercial cDNA library derived from human liver. The forward primer includes an EcoRI restriction site and optimized Kozak sequence and the reverse primer includes a Not1 restriction site following the stop codon:

Forward primer:
(SEQ ID NO: 26)
5′AGCCTCGAGAATTCTAATAGGCCACCATGTCCACTGCGGTCCTGGAAA
ACCCAGGCTTGG 3′
Reverse primer:
(SEQ ID NO: 27)
5′GGAAGCGGCCGCCTACTTTATTTTCTGGAGGGCACTGCAAAGGATTCC
AATTTCACTG 3′.

The PCR product and pDK9-2 were digested with EcoRI and NotI and ligated to generate pDK9-2-PAH. The final size of the pDK-PAH plasmid is 4.3 kb. The nucleic acid sequence of the pDK-PAH vector is provided as SEQ ID NO: 28.

For comparative studies, the same PAH nucleic acid was cloned into a pcDNA vector (InVitrogen). The PCR product and pCDNA6 were digested with EcoRI and NotI and ligated to generate pCDNA6-PAH (SEQ ID NO: 29). The final size of the pcDNA-PAH vector is 6.5 kb.

### Transient Expression Studies

The ability of the pDK-PAH vector to transiently express phenylalanine hydroxylase in eukaryotic cells was then assessed.

293T cells were transfected using 293 CellFectin® according to the manufacturer's instructions. DNA amounts employed for transfection was adjusted for equal molecules given that pcDNA-PAH is 1.51 times larger than pDK-PAH. Transfection 1, 2, 5, 10, 20 or 25 μg of pcDNA-PAH DNA and 0.66, 1.3, 3.3, 6.6, 13.3 or 16.6 μg of pDK-PAH DNA were tested.

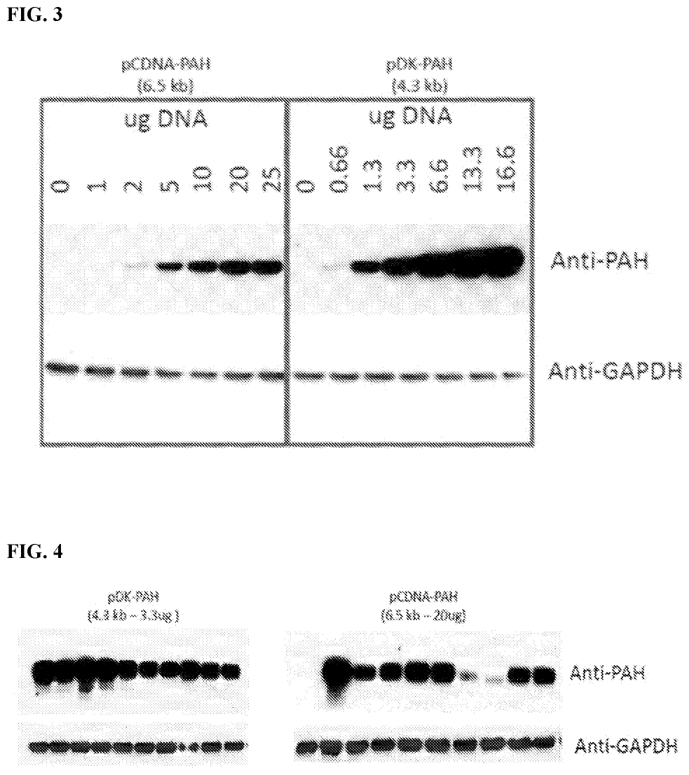

At 48 hours post transfection, the cells were harvested and lysed. The cell lysates were assessed by Western blot using anti-PAH and anti-GAPDH control antibodies. As shown in FIG. 3, the pDK-PAH plasmid expresses significantly higher levels of PAH compared to pcDNA-PAH at comparable levels of the two plasmids.

Stable Integration of the pDK-PAH Plasmid Vector

293T cells were transfected as described above and selected for positive integration of the PAH nucleic acid. 48 hours post transfection, both transfected and untransfected (control) cells were split 1:10 and put under Blasticidin S selection (10 μg/ml final concentration). Cells were kept under selection until all control cells had died, (11 days). 10 Resistant colonies of cells from each of the transfected populations were randomly picked and allowed to expand for 3 weeks under continued Blasticidin S antibiotic selection. Cells were lysed and normalized amounts of each colony were tested for PAH and GAPDH expression as above.

Ten random integration stable clones from each transfection were selected for analysis of PAH expression. As shown in FIG. 4, the pDK-PAH transfected cells exhibited the ability to produce more consistent and stable integration of the PAH nucleic acid compared to pcDNA-PAH transfected cells.

### Example 3. Generation and Characterization of the pDK-Factor VIII-BDD Vector

In this example, the ability of the pDK9 vector to function as an expression vector for larger nucleic acid inserts was assessed by generating a pDK9 vector comprising a nucleic acid encoding B-domain-deleted factor VIII (FVIII-BDD). A description of the methods for the cloning of the nucleic acid encoding FVIII-BDD (about 6 kb) into the pDK9-2 vector is provided.

### Vector Construction

pDK9-2-FVIIIBDD and pcDNA6-FVIIIBDD Assembly

The FVIII-BDD gene (FVIII to Minimal B Domain) was PCR amplified from a commercial cDNA library derived from human liver. The forward primer includes an Xho1 restriction site and an optimized Kozak sequence:

Forward primer:
(SEQ ID NO: 30)
5′AGGCTAGCCTCGAGGTAATAGGCCACCATGCAGATCGAGCTGTCCACC
TGCTTTTTTCTG3′
Reverse primer:
(SEQ ID NO: 31)
5′CAGGGTTGTCCGGGTGATCTCCCGCTGGTGACGCGTGCTGGACACATT
CTTGCCCCAGCT3′.

A second PCR was performed to amplify from the Minimal B Domain (overlap with PCR1) including a Stop codon and NotI site (added in oligo), using the following primers:

Forward primer:
(SEQ ID NO: 32)
5′AGCTGGGGCAAGAATGTGTCCAGCACGCGTCACCAGCGGGAGATCACC
CGGACAACCCTG 3′
Reverse primer:
(SEQ ID NO: 33)
5′GGAAGCGGCCGCTCATCAGTACAGATCCTGGGCCTCACATCCCAGGAC
TTCCATCCTGAG3′.

The PCR1 and PCR2 products were mixed and extended at the two ends by PCR to generate PCR product 3.

The pDK9-2 vector and the product of PCR3 were digested with XhoI and NotI and ligate to generate vector pDK9-2-VFVIII-BDD. The final size of the pDK-FVIII-BDD plasmid vector is 9.0 kb. The nucleic acid sequence of the pDK-FVIII-BDD vector is provided as SEQ ID NO: 34.

For comparative studies, the same FVIII-BDD nucleic acid was cloned into a pcDNA vector (InVitrogen). To generate pCDNA6-FVIIIBDD, pCDNA6 was digested with Kpn1 and blunted. The product of PCR3 was digested with XhoI and blunted. Both insert and vector were then digested with Not1 and ligated to generate pCDNA6-FVIIIBDD (SEQ ID NO: 35). The final size of the pcDNA-FVIII-BDD vector is 11.3 kb. This plasmid vector was difficult to generate due to its large size.

### Transient Expression Studies

The ability of the pDK-FVIII-BDD vector to transiently express FVIII-BDD in eukaryotic cells was then assessed.

293T cells were transfected using 293 CellFectin® according to the manufacturer's instructions. DNA amounts employed for transfection were adjusted for equal molecules of pcDNA-FVIII-BDD and pDK-FVIII-BDD. The pcDNA-FVIII-BDD vector is 1.25 times larger than the pDK-FVIII-BDD vector.

At 5 days post transfection, conditioned medium from the cells was harvested. The conditioned media were assessed by Western blot using anti-Factor VIII C-domain antibodies. As shown in FIG. 5, the pDK-FVIII-BDD plasmid expresses significantly higher levels of FVIIIBDD compared to pcDNA-FVIII-BDD at comparable levels of the two plasmids.

### Example 4. Stable Integration of the pDK-FVIII-BDD Plasmid Vector Using Cas9 Targeted Integration

In this example, stable integration using the Cas9 targeting integration system is described.

Generation of pDK-FVIIIBDD-AAV1 and pDK-PAH-AAV1 Targeting Vectors

Homology targeting versions of the pDK-FVIIIBDD and pDK-PAH vectors to target the AAV1 integration site were generated.

For pDK9-2:

Genomic DNA was prepared from 293T and human Adipose Derived Stem Cells (ADSCs). The homology arms of the AAV1 integration site was PCR amplified from the genomic DNA using primer including the 8 base restriction sites for cloning.

Left Arm PCR:
Forward primer:
(SEQ ID NO: 36)
5′AGCAACGCGATTTAAATTGCTTTCTCTGACCAGCATTCTCTCCCCT
3′
Reverse primer:
(SEQ ID NO: 37)
5′TGAAGATCTCCTGCAGGGCCCCACTGTGGGGTGGAGGGGACAGATAAA
AGTA 3′.
Right Arm PCR:
Forward primer:
(SEQ ID NO: 38)
5′TACTCATGAGGCGCGCCACTACTAGGGACAGGATTGGTGACAGAAAAG
CCCCA 3′
Reverse primer:
(SEQ ID NO: 39)
5′TGATCTGTTTAAACAGAGCAGAGCCAGGAACACCTGTAGGGAAGGGGC
A 3′.

The PCR products were sequenced and found to have the same sequence from the 2 different cell lines used.

The pDK9-2 vector and the PCR product of the Right Homology arm were digested with AscI and PmeI and ligated to generate pDK9-2_AAVS1R (intermediate vector).

The pDK9-2_AAVR1R vector and the PCR product of the Left Homology Arm were digested with SbfI and SwaI and ligated to generate pDK9-2 AAVS1Targeted vector (SEQ ID NO: 40).

To generate the pDK9-2_PAH_AAVS1Targeted vector (SEQ ID NO: 41), the PAH PCR product of Example 2 and the pDK9-2 AAVS1Targeted vector were digested with EcoRI and NotI and ligated.

To generate the pDK9-2_FVIIIBDD_AAVS1Targeted vector (SEQ ID NO: 42), the FVIIIBDD PCR product of Example 3 and the pDK9-2 AAVS1Targeted vector were digested with XhoI and NotI and ligated.

Assembly of AAVS1-Targeted pCDNA6-PAH Vector

The Left Homology Arm was inserted into the SspI site of pcDNA6-PAH (Example 2). The left arm homology arm was amplified as described above, digested with SbfI, blunted, and then digested with SwaI. pcDNA6-PAH was digested with SspI. The digested pcDNA6-PAH vector and the PCR product of the Left Homology arm were ligated to generate pcDNA6-PAH Left (temporary vector).

The Right Homology Arm was inserted into the SapI site of pcDNA6-PAH Left vector. The left arm homology arm was amplified as described above, digested with AscI, blunted, and then digested with PmeI. pcDNA6-PAH Left was digested with SapI and blunted. The digested pcDNA6-PAH Left vector and the PCR product of the Right Homology arm were ligated to generate pcDNA6-PAH_AAVS1Targeted vector (SEQ ID NO: 43).

Assembly of AAVS1-targeted pCDNA6-FVIIIBDD vector

The Left Homology Arm was inserted into the SspI site of pcDNA6-FVIIIBDD (Example 3). The left arm homology arm was amplified as described above, digested with SbfI, blunted, and then digested with SwaI. pcDNA6-FVIIIBDD was digested with SspI. The digested pcDNA6-FVIIIBDD vector and the PCR product of the Left Homology arm were ligated to generate pcDNA6-FVIIIBDD_Left (temporary vector).

The Right Homology Arm was inserted into the BstZ17I site of pcDNA6-FVIIIBDD Left vector. The left arm homology arm was amplified as described above, digested with AscI, blunted, and then digested with PmeI. pcDNA6-FVIIIBDD_Left was digested with BstZ171. The digested pcDNA6-FVIIIBDD_Left vector and the PCR product of the Right Homology arm were ligated to generate pcDNA6-FVIIIBDD_AAVS1Targeted vector (SEQ ID NO: 44).

### Stable Integration of the Targeted Vectors

293T or Human Adipose Derived Stem Cells (hADSC) were transfected with a commercially available plasmid DNA expressing Cas9 and a guide RNA targeting the AAV1 integration site, HCP-AAVS1-CGO2 from Genecopia and the homology targeted versions of the expression vectors. 293T Cells were transfected with 293CellFectin and 1 μg of the HCP-AAVS1-CGO2 plasmid and with or without 10 μg of pcDNA-PAH AAV1STargeted plasmid or 1 μg HCP-AAVS1-GC02 with or without 10 μg pcDNA-FVIIIBDD-AAVS1Targeted plasmid, or 1 μg HCP-AAVS1-GC02 and with or without 7.7 μg pDK-PAH-AAVS1Targeted plasmid or 1 μg HCP-AAVS1-GC02 and with or without 8.5 μg pDK-FVIIIBDD-AAVS1Targeted plasmid.

hADSC cells were transfected in a similar manner to the 293T cells, however, instead of 293CellFectin, Lipofectamine 3000 was used.

Cells were selected for antibiotic resistance and 96 clones were selected for each combination variant. Antibiotic resistance was provided by the expression vector, so without expression vector, no cells survived selection.

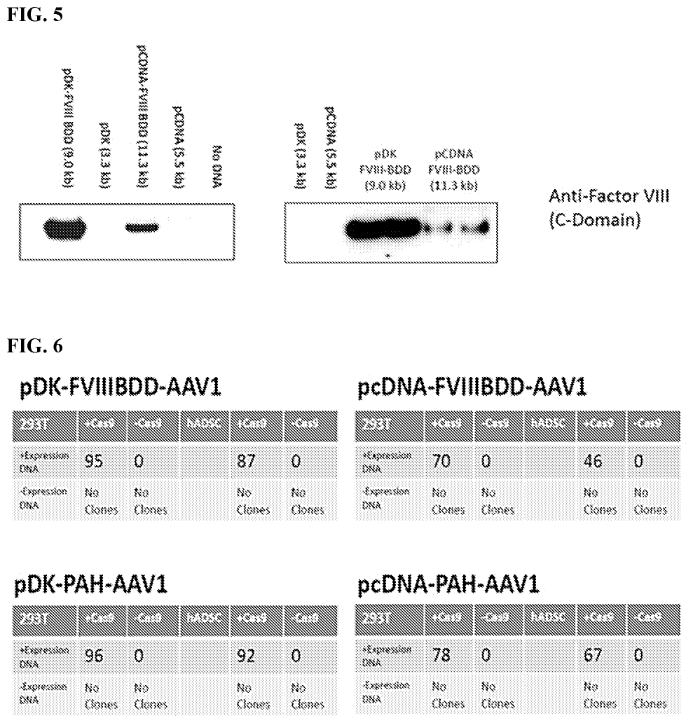

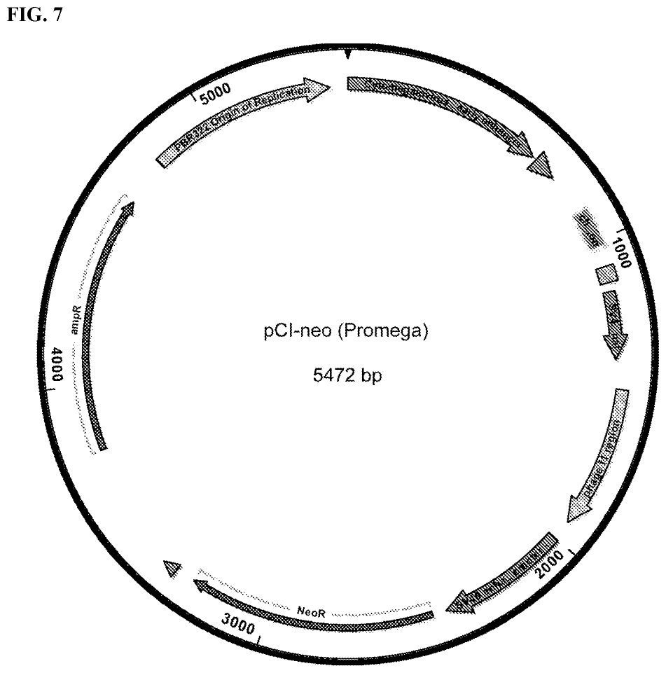

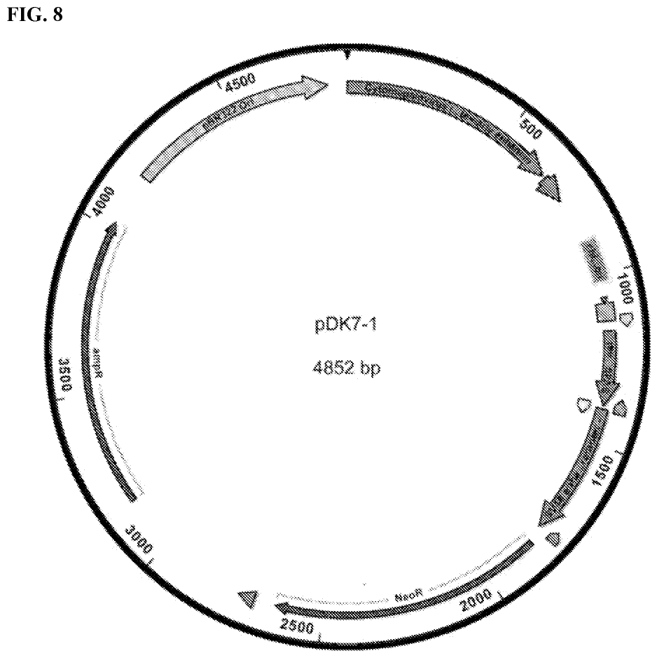

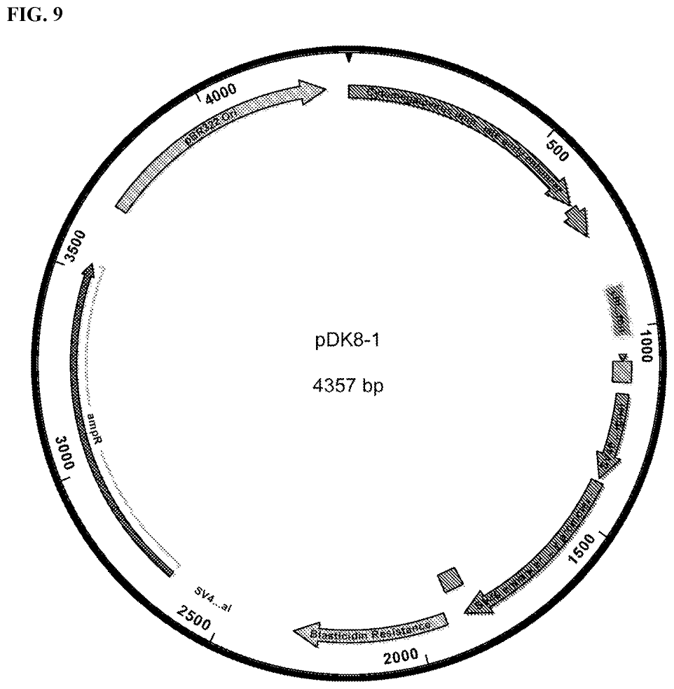

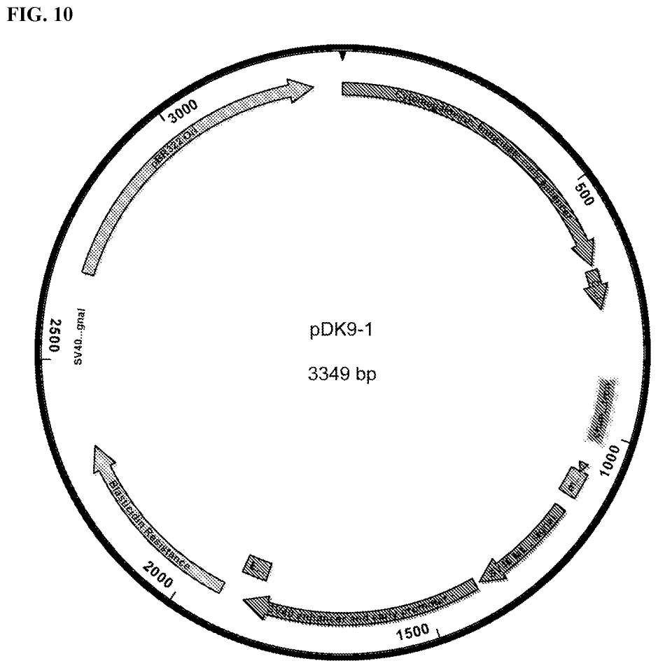

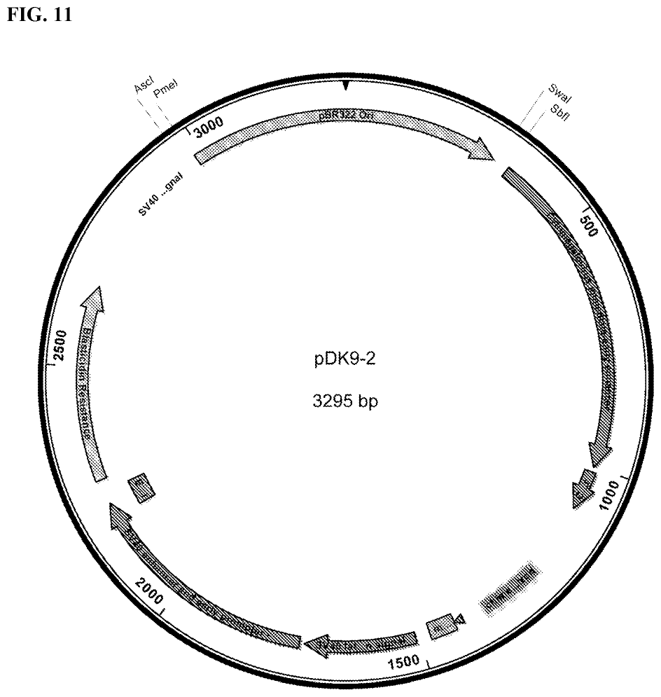

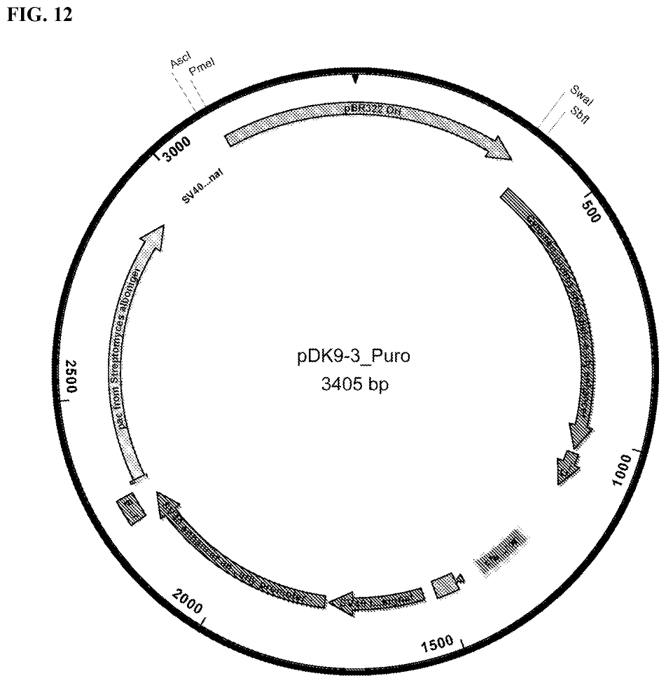

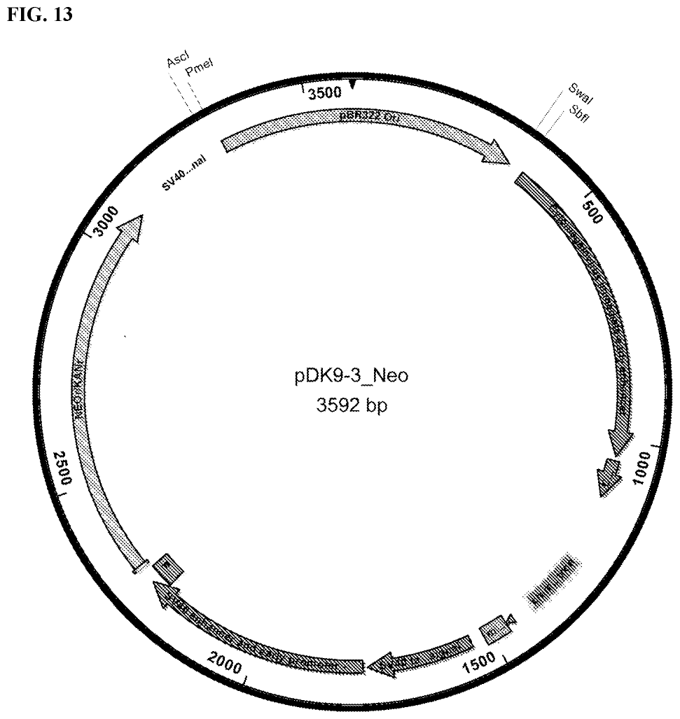

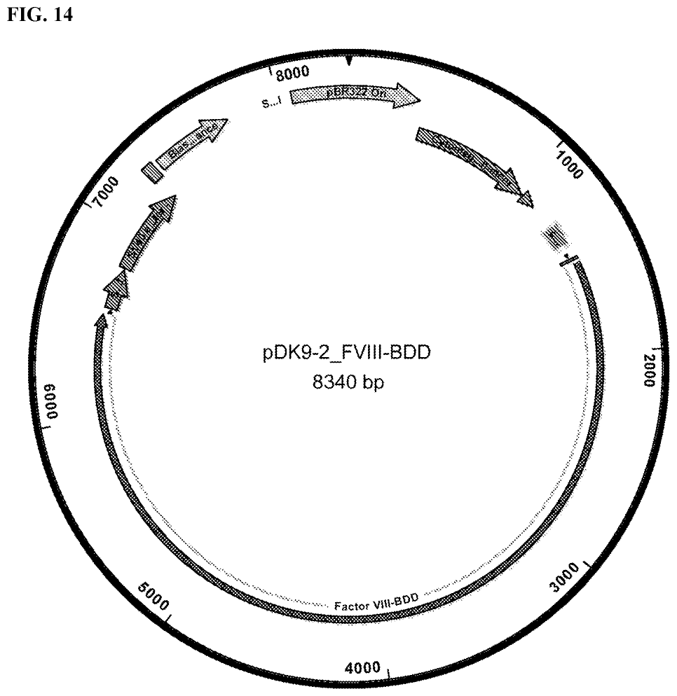

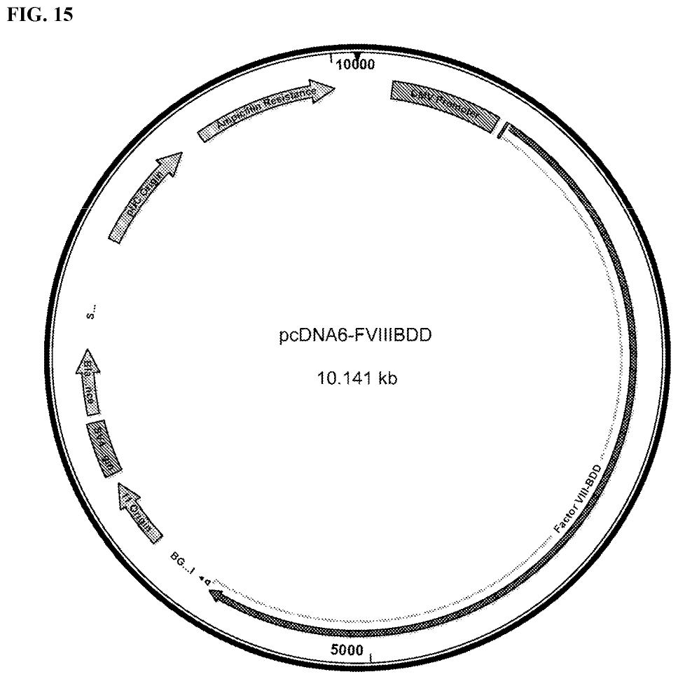

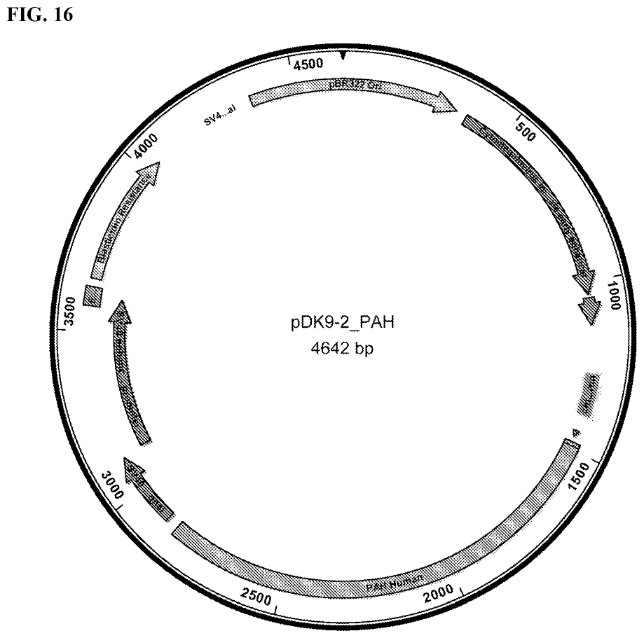

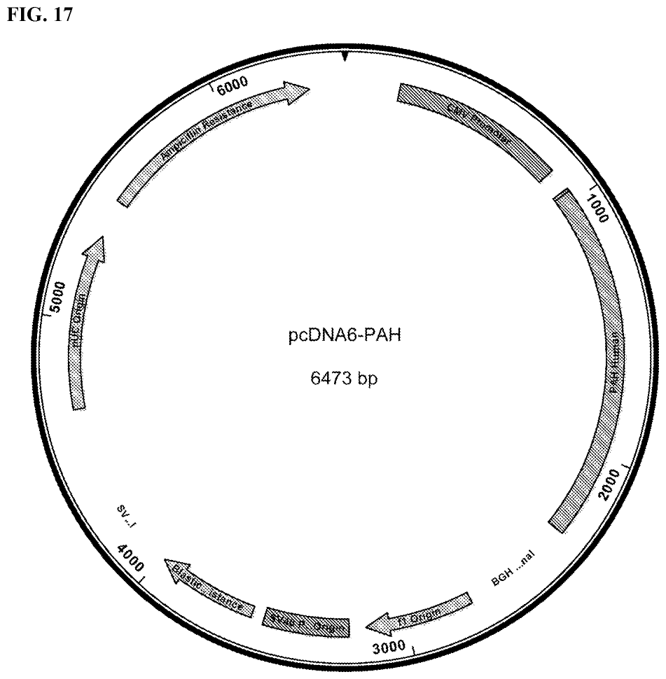

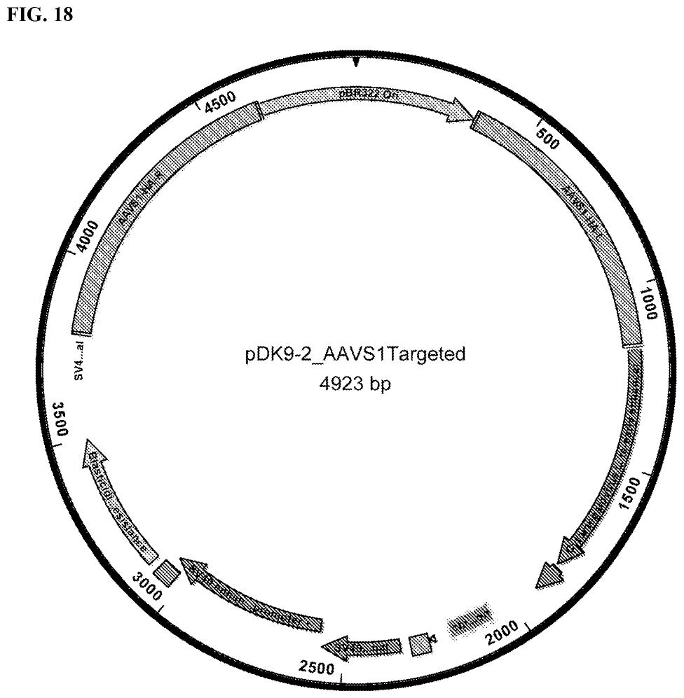

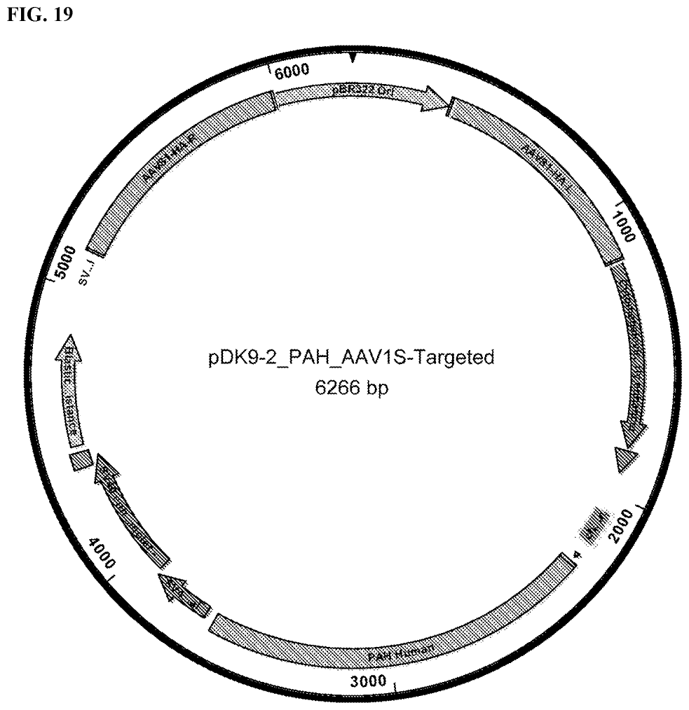

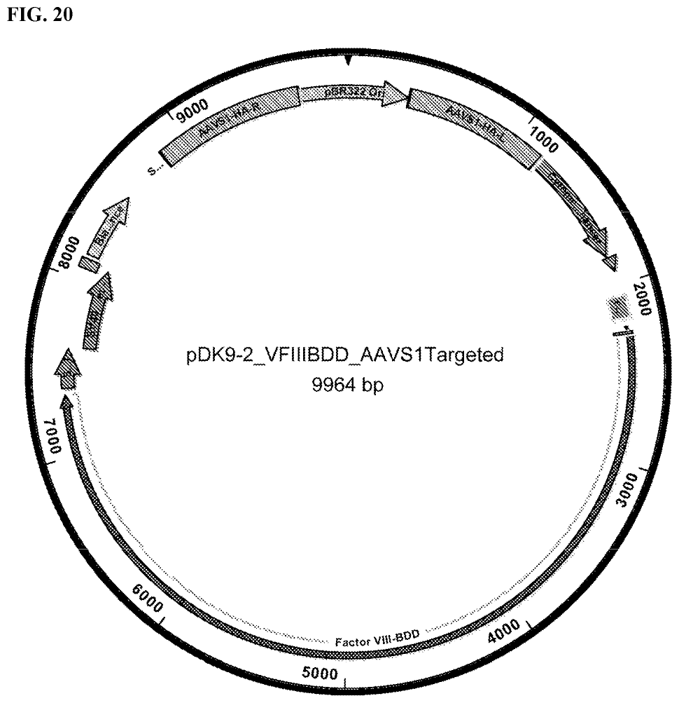

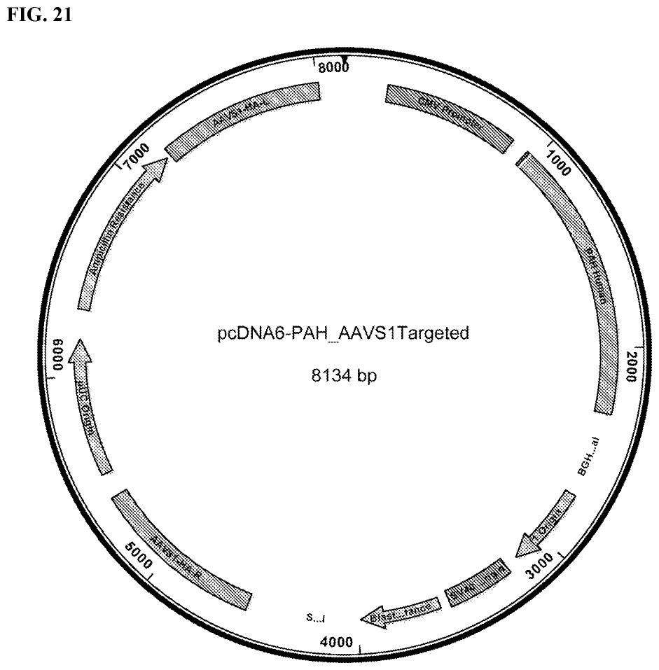

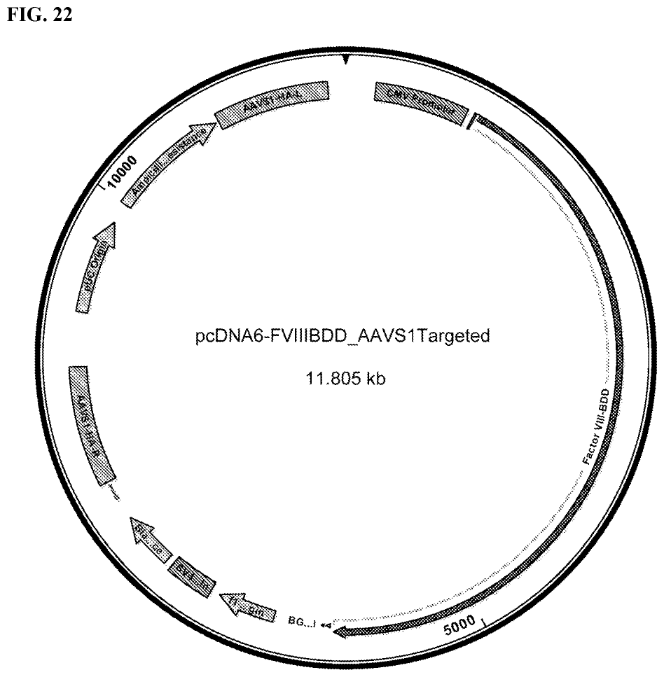

Genomic DNA was prepared for each clone and integration was determined by polymerase chain reaction amplification (PCR) across the junction site on both 5′ and 3′ sides. One genomic primer outside of the homology region and one primer from vector derived sequence were employed for the PCR reaction. Cells were considered positive when both sides produced an amplification product indicating that there was targeted integration. The results of the target integration are provided in FIG. 6. As show in FIG. 6, both the pDK-FVIIIBDD-AAV1 and pDK-PAH-AAV1 generated significantly higher success rates for targeted integration over the pcDNA vectors.

Selection using a single selectable marker under control of a hybrid promoter required much higher levels of antibiotic in bacterial cells compared to human cells (i.e., eukaryotic cells). For eukaryotic cells, blasticidin S at 1-10 μg/ml was sufficient for selection of cells that had successfully taken up the vector, and puromycin at 1-5 μg/ml was sufficient for selection of cells that had successfully taken up the vector. For prokaryotic cells, blasticidin S at 100 μg/ml was sufficient for selection of cells that had successfully taken up the vector, and puromycin at 50-100 μg/ml was sufficient for selection of cells that had successfully taken up the vector.

Selection using a single selectable marker under control of a hybrid promoter was different from traditional antibiotic selection. Bacterial cells did not die immediately in response to the antibiotic if they had not taken up the vector. Instead, a thin layer or lawn of bacterial cells was present along with strong colonies of bacterial cells that had taken up the vector. Cells picked from the thin layer failed to grow in liquid culture. This result did not depend on the type of bacteria used.

It should be noted that TB medium worked better than LB medium for culturing. In general, the yield of cells that had successfully taken up the vector was high.

### Example 5. Method for Swapping the Expression Promoter in pDK9-2

The pDK9-2 vector is digested with HindIII and BgIII to remove the CMV enhancer and promoter. Any suitable alternative promoter can be inserted in place of the CMV enhancer and promoter. Non-limiting examples include: Promoter of the Beta-Actin gene from human, mouse, or chicken, the promoter of the Ubiquitin C gene, or the promoter of the Thymidine Kinase gene from Herpes Virus.

### Example 6. Method for Swapping the Poly a Signal in pDK9-2

The pDK9-2 vector is digested with NotI and TspGWI to remove the SV40 late poly A signal. Any suitable alternative Poly A signals can be inserted in place of the SV40 late poly A signal. Non-limiting examples include: Growth Hormone Poly A signal from bovine and synthetic Poly A signals.

### Example 7. Method for Swapping the PBR322 Origin of Replication in pDK9-2

The pDK9-2 vector is digested with AscI and SbfI to remove the PBR322 Origin of Replication. Any suitable alternative Origin of Replication can be inserted in place of the PBR322 Origin of Replication. Non-limiting examples include: P15A Low copy number Origin of Replication or a pUC Origin of Replication

### Example 8. pDK-Streamline Vectors

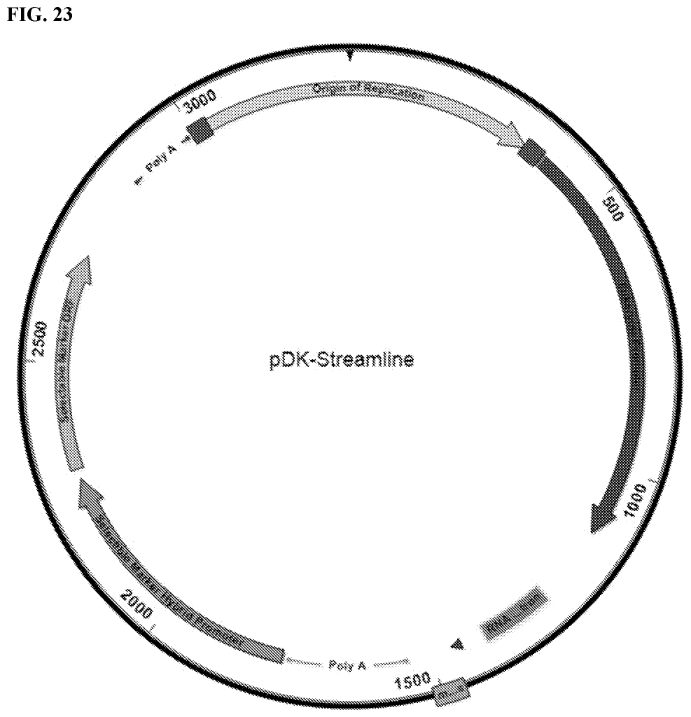

The pDK-Streamline vector (FIG. 23) includes the following structural components: an expression vector main promoter, an expression vector selectable marker, rare 8 base restriction sites for homology arms, an RNA stabilizing splice site to increase protein expression, a T7 promoter for bacterial or cell-free expression, and a poly A signal sequence for RNA stability. The backbone of the pDK Streamline vector may be 3.6 kb or less.

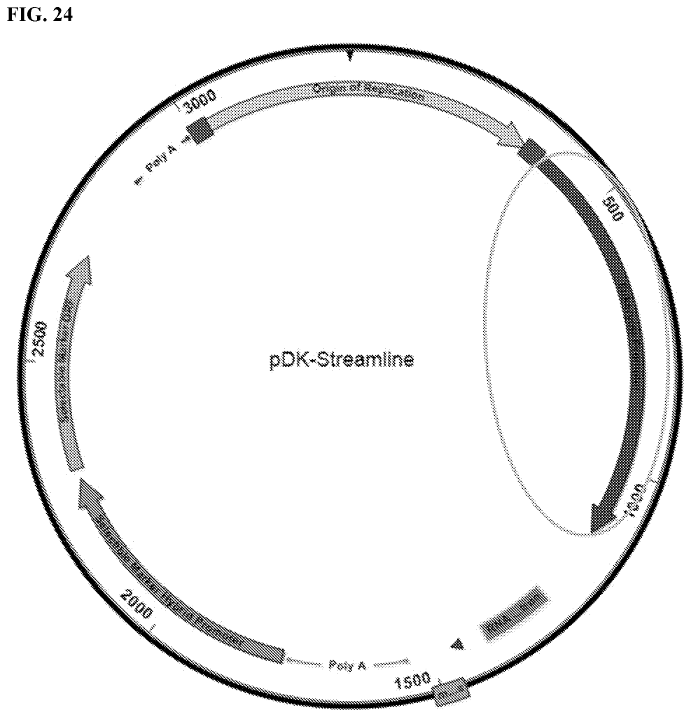

Non-limiting examples of the expression vector main promoter (FIG. 24) include a CMV enhancer and promoter, a Chicken BetaActin promoter, and a Ubc promoter. Each of these promoters offers a unique advantage. The CMV enhancer and promoter is a viral promoter useful for achieving high levels of protein expression, while the Chicken BetaActin promoter is considered one of the strongest “natural” promoters. The Ubc promoter is a promoter expressing a component of the Ubiquitin system, which is active in nearly every cell type. As is well known in the art, selecting a suitable promoter to drive gene expression is critical for the success of cell-based therapies. The pDK-Streamline vector is designed to make changing the main promoter easy through the use of flanking restriction sites.

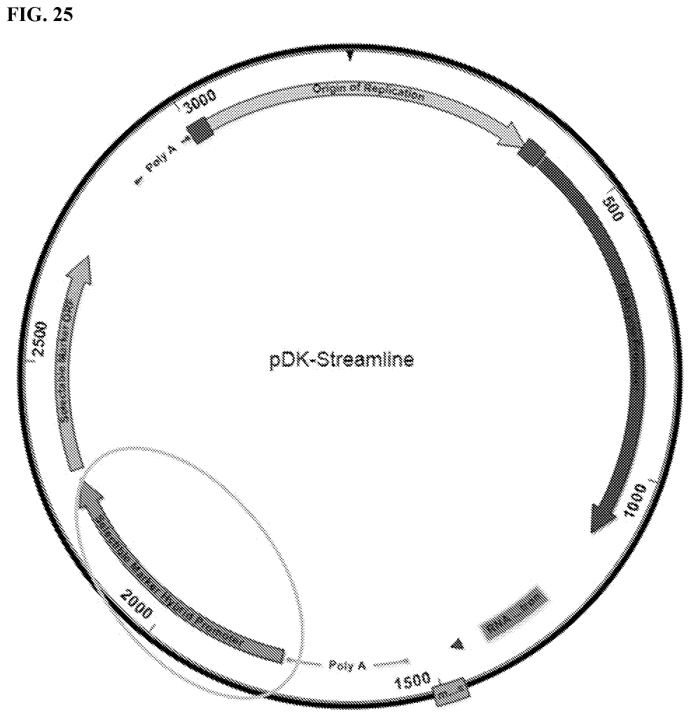

The expression vector selectable marker has a small size due, in part, to the elimination of a separate selectable marker for bacteria. By creating a hybrid promoter (FIG. 25) with activity in both prokaryotes (bacteria) and eukaryotes (mammalian cells) there is antibiotic resistance in both settings from a single gene. The pDK-Streamline vector may include one of 3 of selectable markers: blasticidin S deaminase, puromycin-N-acetyltransferase, and neomycin phosphotransferase. It is contemplated that other selectable markers may be useful.

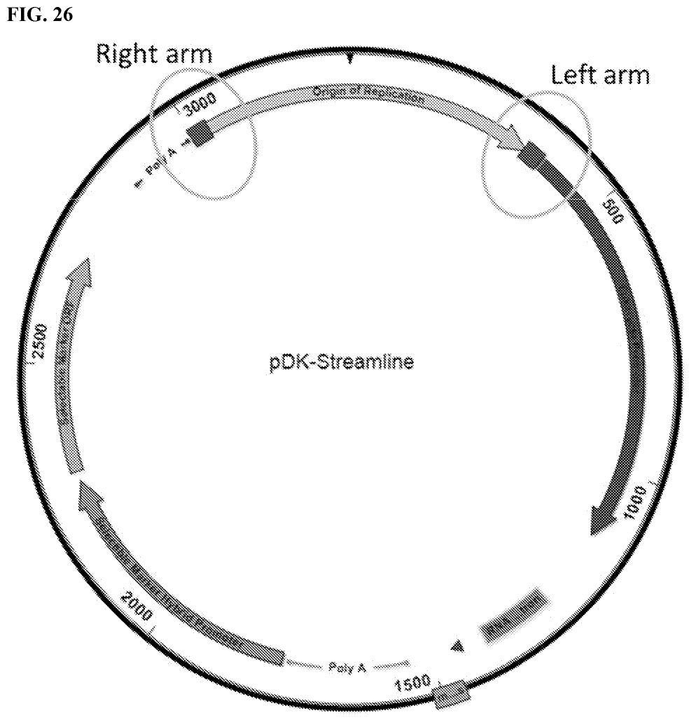

Homology arms are inserted on either side of the expression cassette (FIG. 26). Each side is flanked by two 8-base restriction sites (FIG. 26). 8-base cutters are extremely rare making it very likely that they will be unique in the vector regardless of the gene of interest or homology arms. In the rare event that one, or more, of these sites are somewhere else, on each side there is an 8-base blunt cutter for insertion of a blunt fragment from restriction digest with blunt enzymes, restriction digest followed by end polishing or a PCR fragment. The left arm, located just in front of the main promoter (e.g., CMV), has SwaI (Blunt) on one side and SbfI on the other side. The right arm has AscI on one side and PmeI (Blunt) just after the Poly A signal (FIG. 26). This organization allows for easy exchange of homology arms in the pDK-Streamline vector.

Placement of the homology arm insertion sites on either side of the (high copy number) bacterial origin of replication ensures that the origin would not be included as part of the template for the cell to insert into the genome, thereby minimizing unexpected effects. The origin also acts as a convenient place to linearize the vector, if desired.

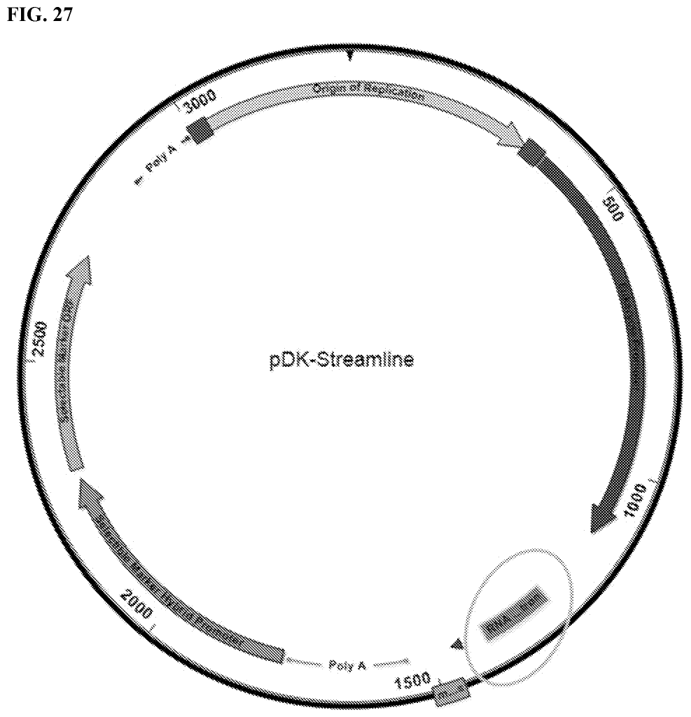

Allowing RNA to be spliced has been shown to increase the stability of the RNA. RNA is inherently unstable and the longer it is intact the greater the amount of protein that can be expressed. Most protein expression Open Reading Frames (ORF) are derived from cDNA or DNA sequences where all of the introns have been removed, mainly in an effort to reduce the size of sequence. Adding in an artificial splice site can enhance RNA stability. pDK-Streamline includes an artificial splice site that enhances RNA stability and allows for increased protein expression (FIG. 27).

Further, the artificial splice site also creates a space for an additional bacterial expression cassette, if desired. For example, a more traditional bacterial resistance marker could be inserted in the artificial splice site and it would act as a “filler sequence” that would be spliced out of the message when inside of a eukaryotic cell.

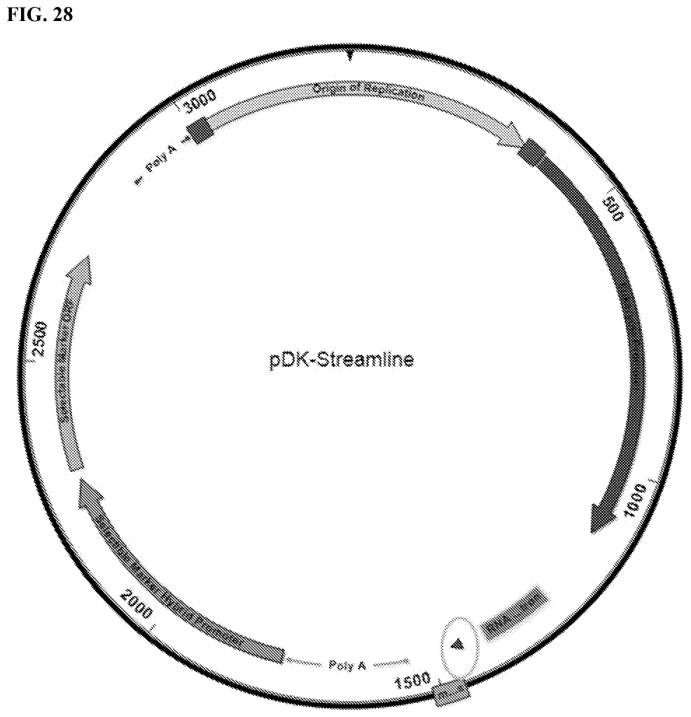

The pDK-Streamline vector includes a T7 promoter just upstream of the multiple cloning site (FIG. 28). The presence of a T7 promoter allows for several benefits. Firstly, the T7 promoter provides a convenient priming site for sequencing. Secondly, it allows for in-vitro transcription and translation (cell free protein expression). Thirdly, it permits bacterial expression of the protein of interest without using a separate vector.

### Example 9. pDK-Streamline Vector Production and Use

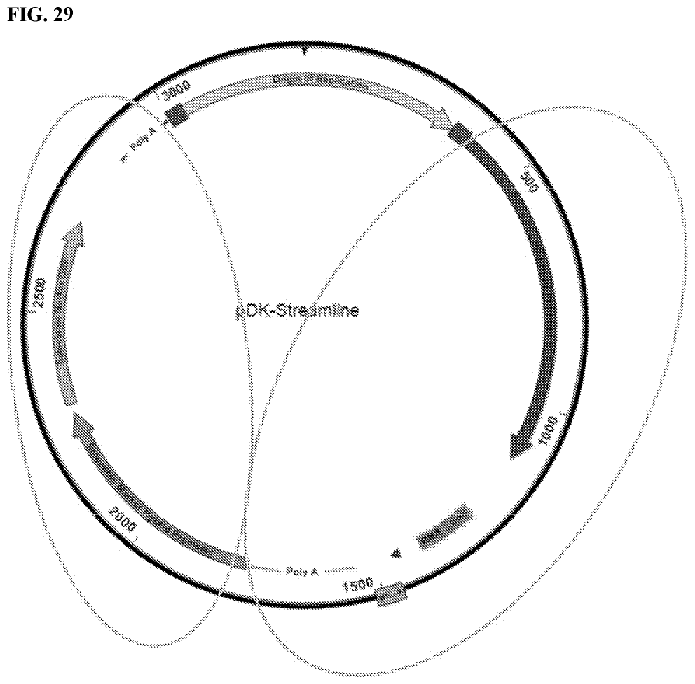

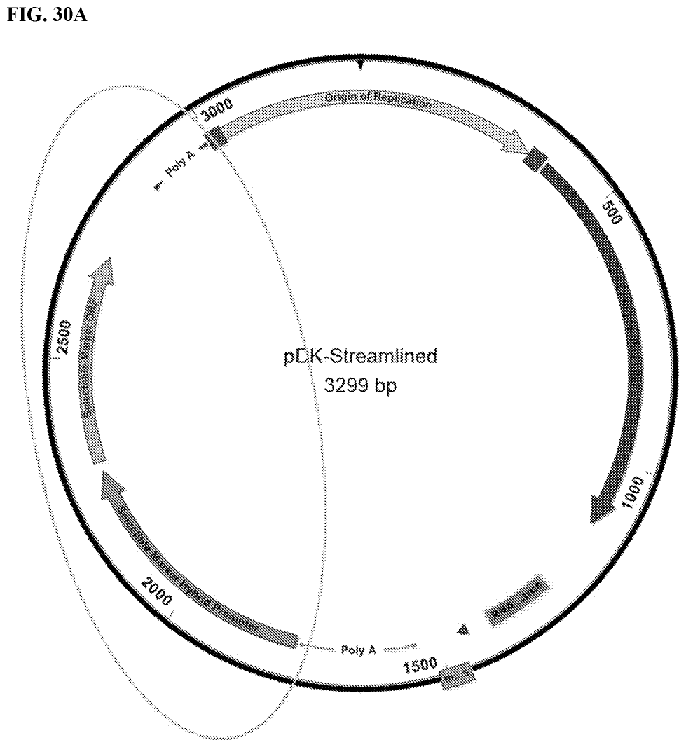

There are two major steps to make a DNA vector for protein expression: 1) creation of the vector with the expression cassette and 2) amplifying the new vector, typically by using bacterial hosts. The “expression cassette” is all of the pieces needed to allow for protein expression. Typically, the expression cassette will include: 1) a promoter, 2) a kozac initiation sequence, 3) the cDNA of the gene to be expressed, 4) and a poly-adenylation signal sequence. FIG. 29 shows the two expression cassette parts of the pDK-Streamline vector. Once the vector is assembled, the DNA vector is amplified in bacterial and purified for use.

For amplification the vector needs an origin of replication (a sequence that drives the bacterial DNA replication) and a gene that usually expresses resistance to an antibiotic (a selection marker). For amplification, the DNA vector forced into a suitable bacterial host, which may be accomplished using methods well-known in the art. The bacteria is then spread on a nutritive, solid, medium with the selection antibiotic (LB Agar). Only bacteria that have taken up the vector, and are thus able to express resistance to the antibiotic are able to grow. Approximately 24 hours later there will be “colonies” of bacteria clones with the vector. One or more of the colonies are separately transferred to a liquid medium, also with antibiotic, for continued expansion. Approximately, 24 hours later the bacteria are lysed and the DNA vector is purified for other uses.

This general method is also used to select mammalian cells that have been transfected or edited with such a vector. First, vector with selection marker is introduced into a mammalian cell. Second, antibiotic is added to kill cells that did not take up vector. Third, cells that survive the selection are expanded.

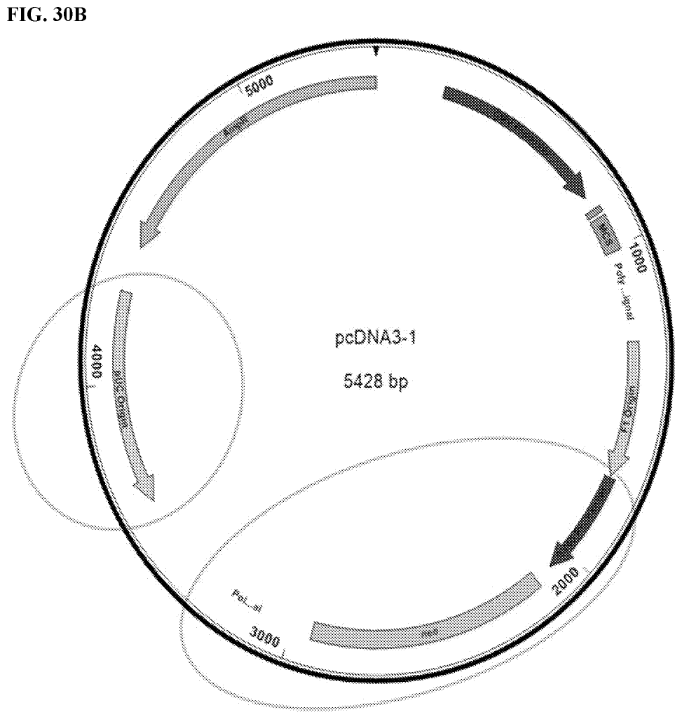

Legacy vectors (e.g., pcDNA3-1 by Invitrogen) would have a separate, bacteria only, selection marker, commonly resistance to ampicillin, kanamycin, tetracycline, etc (FIG. 30B). Legacy vectors would have a separate selection marker for mammalian cells, such as resistance to puromycin, blasticidinS, neomycin, etc (FIG. 30B). The markers would be expressed as separate expression cassettes (FIG. 30B). These vectors are inherently larger than pDK-Streamline vectors due to the need for two separate expression cassettes (FIG. 30A-30B).

pDK-Streamline vectors combine the selection marker for both bacteria and mammalian cells into one expression cassette by creating a promoter that is able to function in both (FIG. 30A). Promoters are limited to working in either bacteria or eukaryotes, like mammalian cells. By arranging and fusing two separate promoters into one expression cassette, the pDK-Streamline vector is able to use a single selection marker in both bacteria and eukaryotes.

Putting the bacterial and mammalian selection under one expression cassette has not been done before, so antibiotics like puromycin and blasticidin S are not typically used for the bacterial selection. A kit of parts could include growth medium, for example LB Agar plates or liquid medium, with puromycin or blasticidin S already in them. For example, a kit with pDK-SL1Blast could have a LB Agar plates containing blasticidin S, or a kit with pDK-SL1Puro could have LB Agar plates containing puromycin, etc. Antibiotic selection plates may be included with the pDK-Streamline vector in a kit. The growth medium (e.g., antibiotic selection plates (e.g. agar plates) or liquid medium) may be formulated specifically for growth and selection of prokaryotic cells. The growth medium (e.g., antibiotic selection plates (e.g., agar plates) or liquid medium) may be formulated specifically for growth and selection of eukaryotic cells.

Another feature the pDK-Streamline vector has is the ability to insert homology arms before and after the expression cassette. Homology arms are required when you want to insert the expression cassette in a specific genomic site, in combination with CRISPR, for example.

A typical process for genomic editing including CRISPR proceeds as follow: the (1) CRISPR complex makes a double stranded break at a specific site in the genome; (2a) the cell recognizes the genomic damage and repairs it, either by removing a small amount of the sequence around the break and then ligating it back together; or (2b) the cell uses the other chromosome as a template to repair the break to have the same sequence as that chromosome.

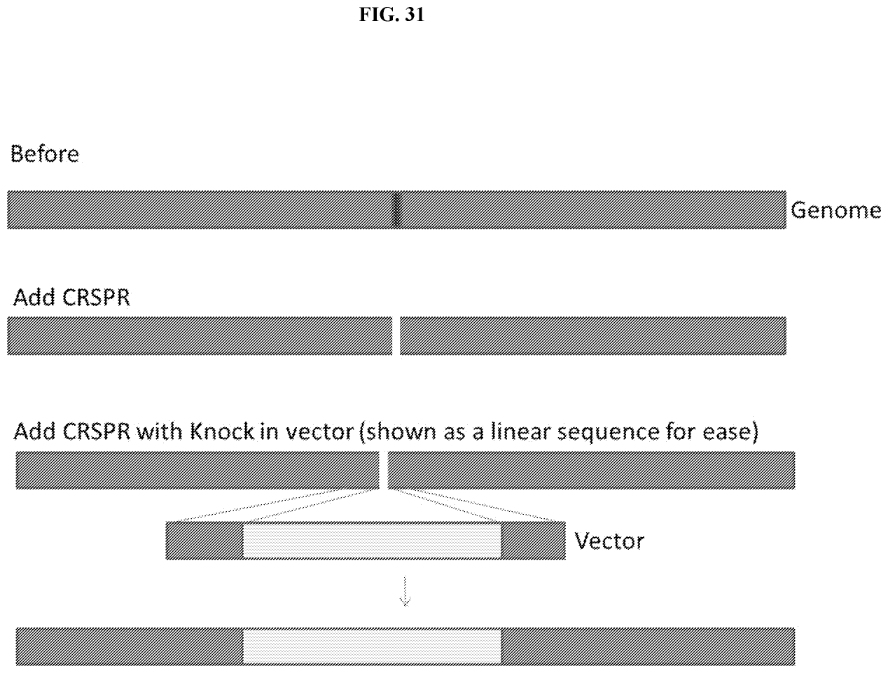

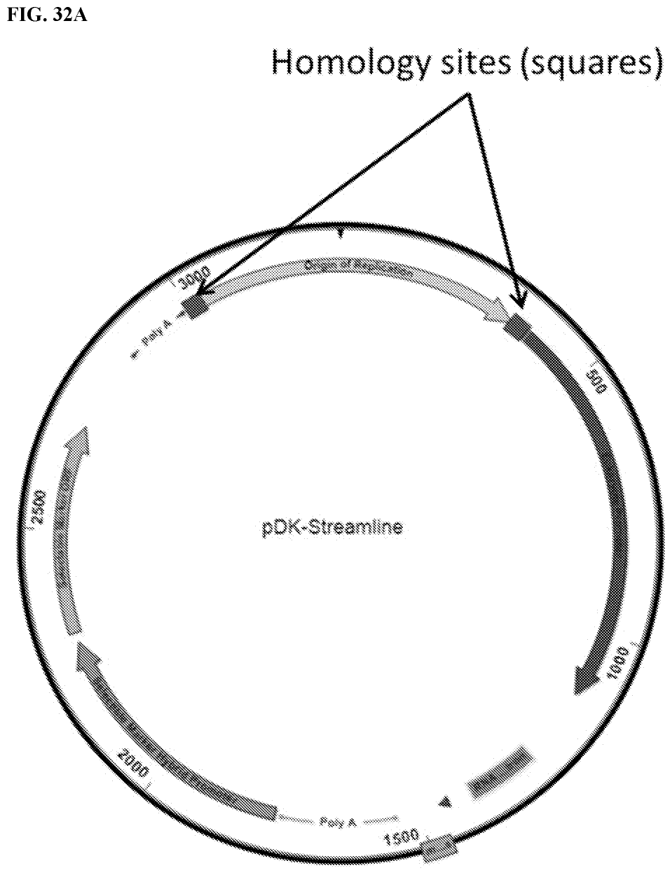

2a above leads to knock-out of the gene as the sequence will be disrupted and likely out of frame. 2b above can be exploited to change the sequence to a preferred sequence. If the cell is flooded with an alternative sequence with homology (identical sequence) on either side of the double strand break, the cell could use that as the template during repairs and introduce that sequence instead (FIGS. 31A-31B). This is called “knock-in” (vs. “knock out” when the gene sequence is disrupted and rendered non-functional).

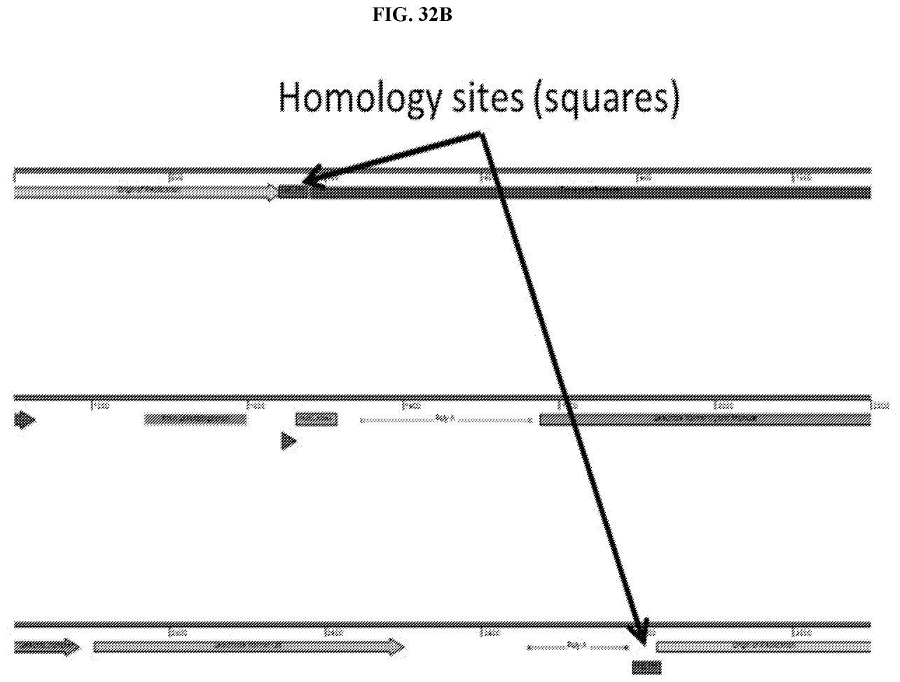

The homology arm insertion sites are positioned to be just before and just after the expression cassettes for the gene of interest and the selection marker (FIG. 32). These sites are bounded with restriction sites for rare cutting enzymes so that the homology arms can be inserted easily and directionally (homology arm has to be in the same direction as the genome). Carefully positioned restriction sites allow for easy insertion and easy change of homology arms.

Enzyme blends for each homology arm and even a blend to linearize the vector by cutting out the bacterial origin of replication can be included in a kit which includes the pDK-Streamline vector. Vectors are frequently “linearized” or cut with a restriction enzyme(s) to increase the chance of integration as well as to remove any sequences that could be detrimental if they were inserted.

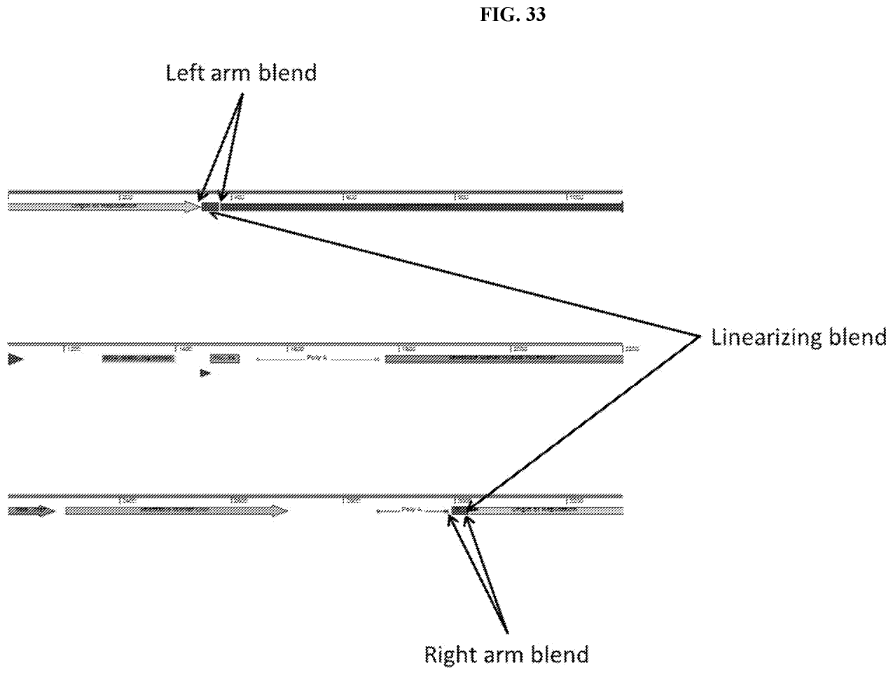

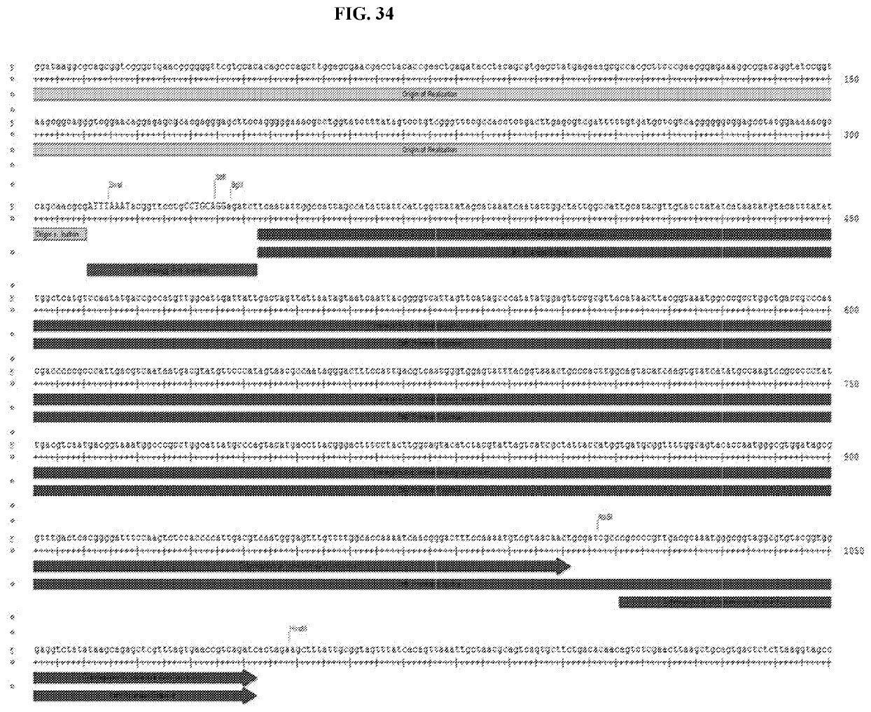

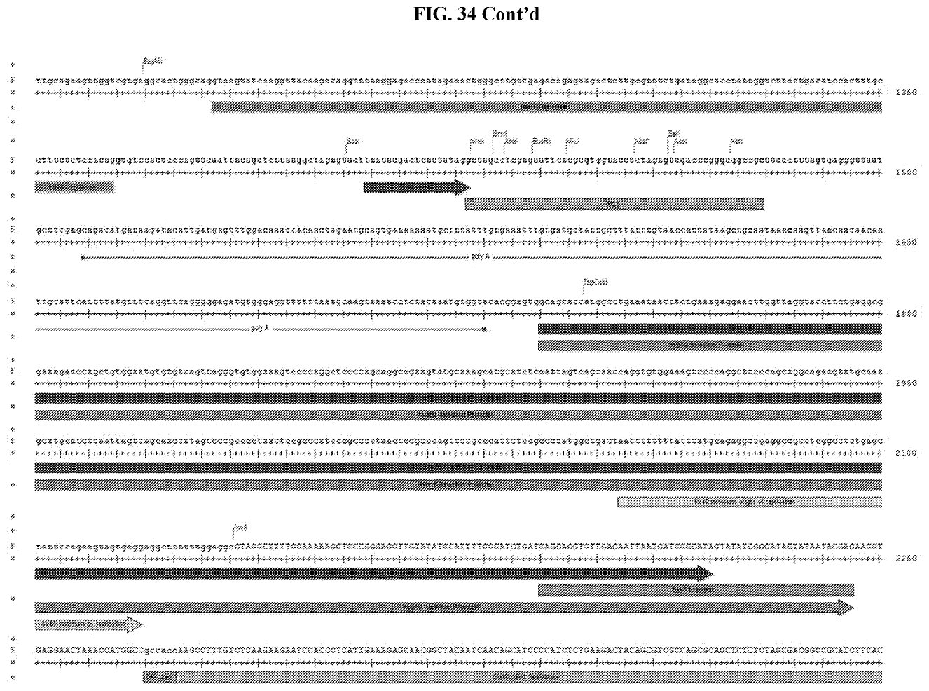

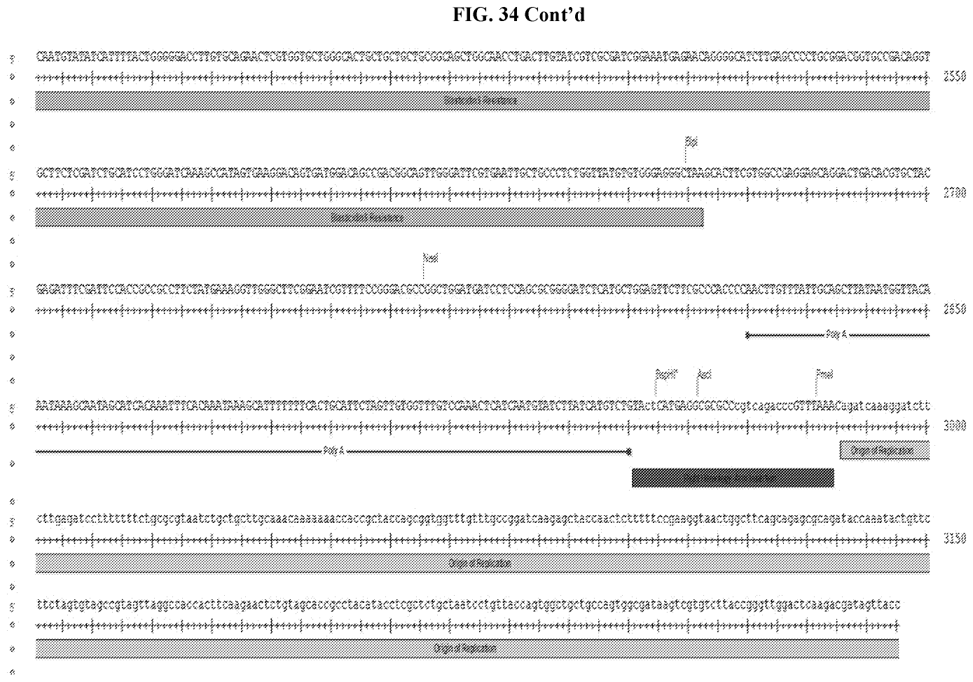

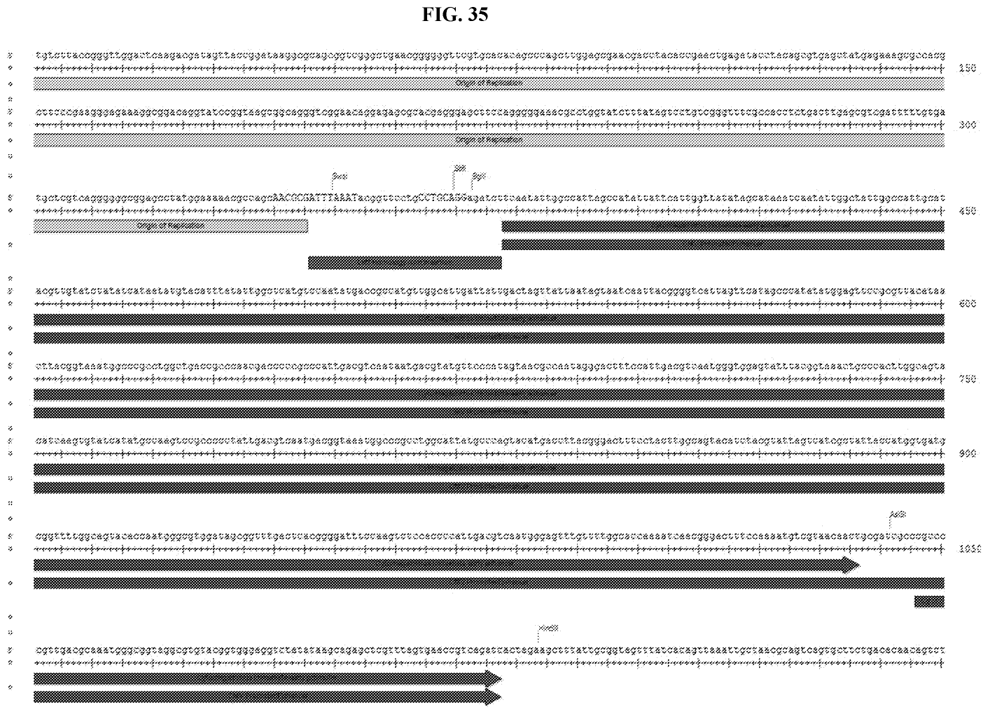

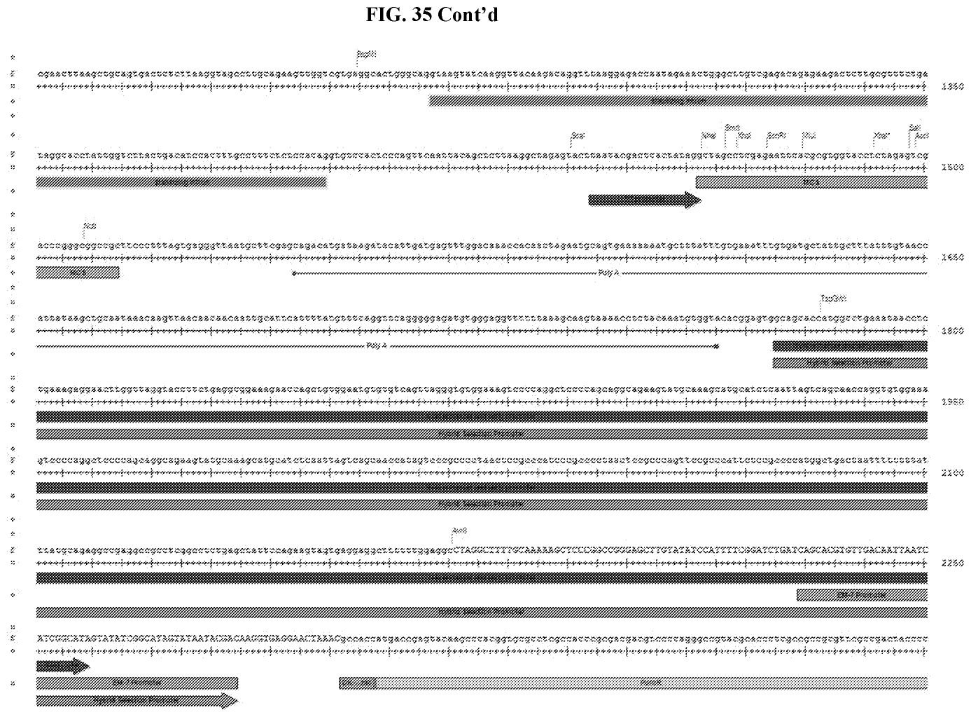

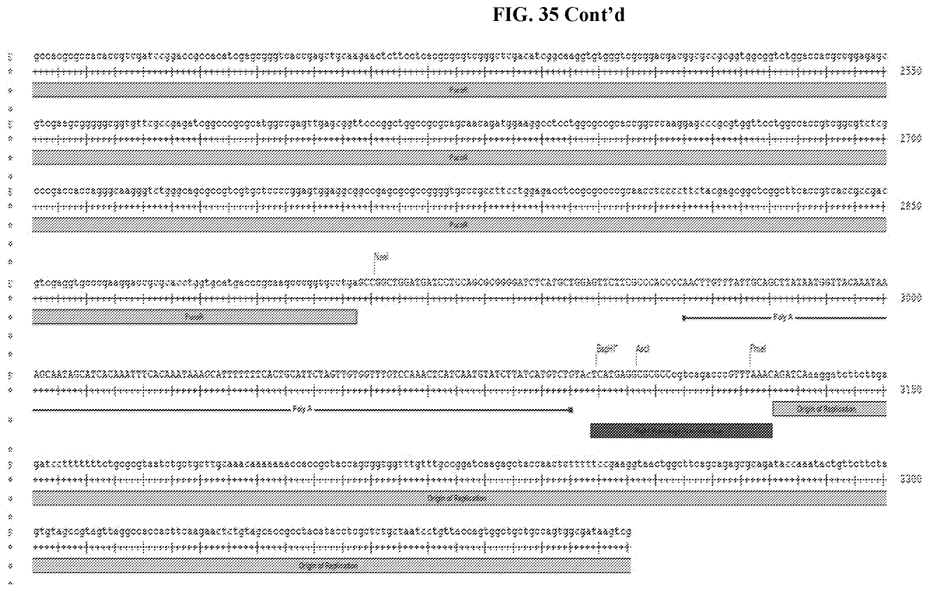

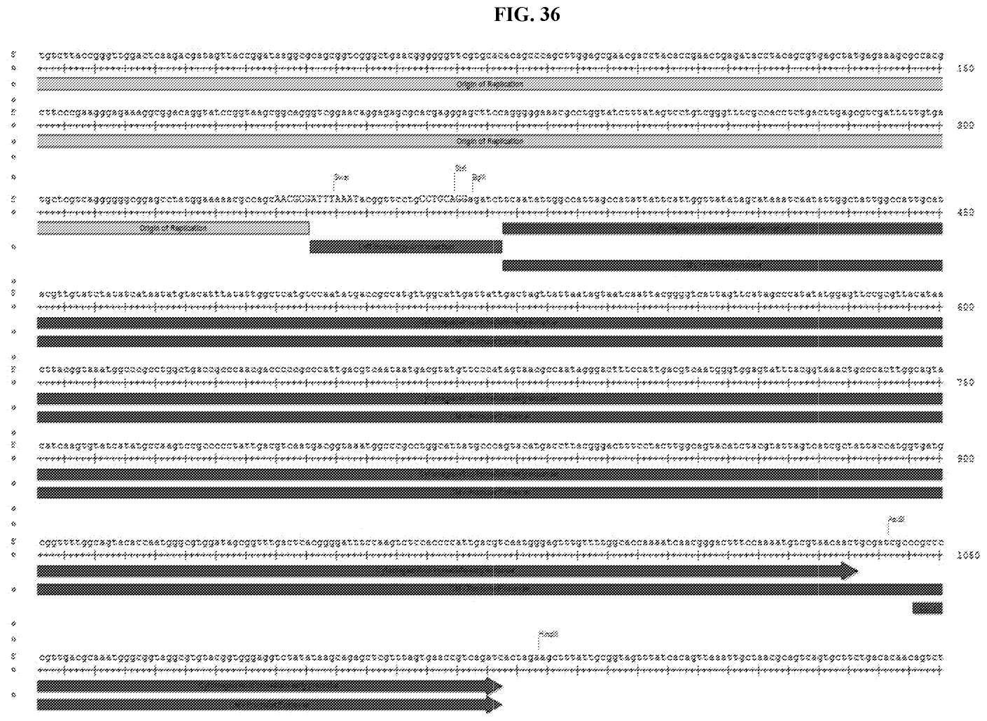

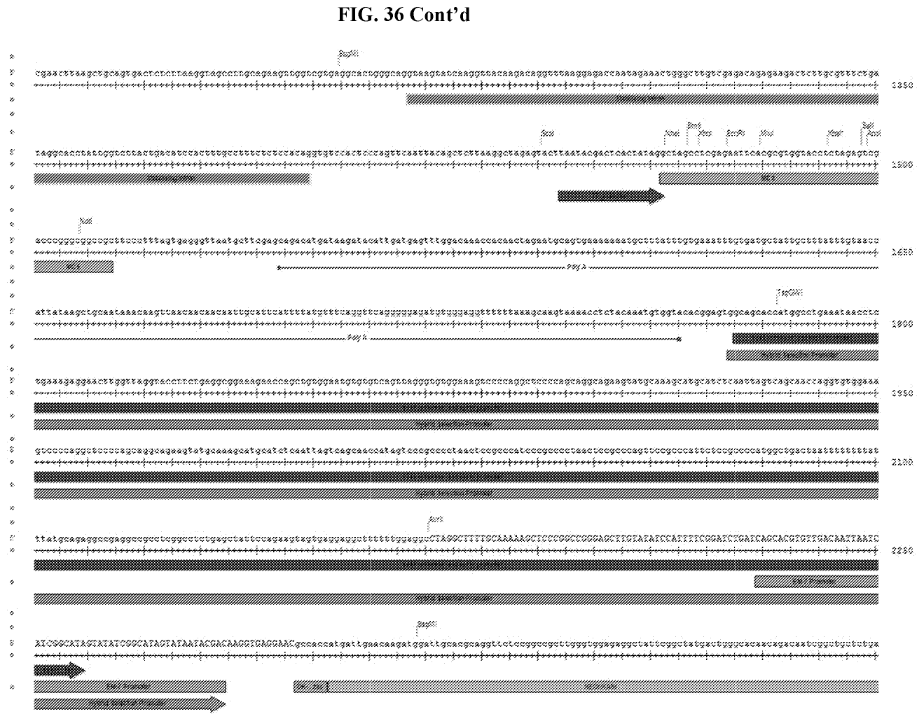

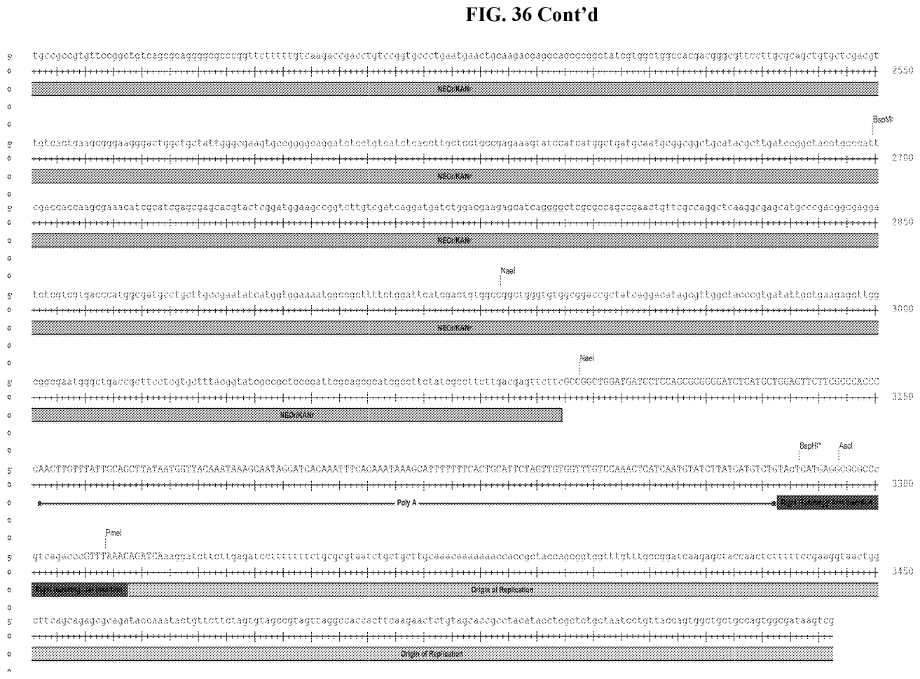

It is contemplated that there could be three different blends: one for the left arm, one for the right arm and one with the two enzymes that cut closest to the origin of replication (FIG. 33). While the enzymes used to cut the restriction sites, as described above, are commercially sold, a blend of the commercially available restriction enzymes is not available. Such a blend is attractive to users since it would reduce errors (adding only one enzyme would open the vector but it would not allow for insertion) and also make it more convenient.

Example 9 demonstrates the technical advantages and ease of use of the pDK-Streamline vector. Further, this Example illustrates the potential for including the pDK-Streamline vector with other components useful for amplifying the vector (e.g., including pre-made antibiotic agar plates) or making modifications to the vector (e.g., changing homology arms using enzyme blends) in, for example, a kit.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### P EMBODIMENTS

### Embodiment P1

A plasmid vector comprising:

(a) a prokaryotic origin of replication;

(b) a eukaryotic promoter suitable for expression of one or more transgenes;

(c) a multiple cloning site for insertion of the one or more transgenes; and

(d) a nucleic acid encoding a selectable marker operably linked to a eukaryotic and a prokaryotic promoter, wherein the selectable marker is suitable for both prokaryotic and eukaryotic selection;

wherein the vector is less than 3.6 kilobases in length.

### Embodiment P2

The plasmid vector of embodiment P1, wherein elements (a) through (d) are arranged sequentially in the 5′ to 3′ direction of the plasmid.

### Embodiment P3

The plasmid vector of embodiment P1 or P2, further comprising an upstream homology arm insertion site located between elements (a) and (b) and a downstream homology arm insertion site.

### Embodiment P4

The plasmid vector of embodiment P3, the downstream homology arm insertion site located after element (d).

### Embodiment P5

The plasmid vector of any one of embodiments P1-P4, further comprising a synthetic splice site between elements (b) and (c) that enhances stability of RNA transcribed from the eukaryotic promoter of (b).

### Embodiment P6

The plasmid vector of any one of embodiments P1-P5, further comprising poly A sequences following the multiple cloning site of (d).

### Embodiment P7

The plasmid vector of any one of embodiments P1-P6, further comprising an additional promotor upstream of the multiple cloning site of (d) for in vitro expression of the one or more transgenes.

### Embodiment P8

The plasmid vector of embodiment P7, wherein the additional promotor for in vitro expression is a T7 promoter.

### Embodiment P9

The plasmid vector of any one of embodiments P1-P8, wherein the origin of replication of (a) is selected from the group consisting of pBR322, pMB1, p15A, pACYC184, pACYC177, ColE1, pBR3286, p1, pBR26, pBR313, pBR327, pBR328, pPIGDM1, pPVUI, pF, pSC101 and pC101p-157.

### Embodiment P10

The plasmid vector of embodiment P9, wherein the origin of replication of (a) is pBR322 Ori.

### Embodiment P11

The plasmid vector of any one of embodiments P1-P10, wherein the eukaryotic promoter of (b) is selected from the group consisting of a cytomegalovirus (CMV) promoter, the promoter of the Beta-Actin gene from human, mouse, or chicken, the promoter of the Ubiquitin C gene, and the promoter of the Thymidine Kinase gene from Herpes Virus.

### Embodiment P12

The plasmid vector of embodiment P11, wherein the eukaryotic promoter of (b) is a cytomegalovirus (CMV) promoter.

### Embodiment P13

The plasmid vector of any one of embodiments P1-P12, wherein the selectable marker is selected from the group consisting of an antibiotic resistance gene, a fluorescent protein, and an enzyme.

### Embodiment P14

The plasmid vector of embodiment P13, wherein the selectable marker is an antibiotic resistance gene.

### Embodiment P15

The plasmid vector of embodiment P13, wherein the selectable marker is blasticidin S deaminase.

### Embodiment P16

The plasmid vector of embodiment P13, wherein the selectable marker is a fluorescent protein.

### Embodiment P17

The plasmid vector of embodiment P16, wherein the fluorescent protein is a near infrared fluorescent protein.

### Embodiment P18

The plasmid vector of any one of embodiments P1-P17, wherein the nucleic acid encoding the selectable marker is operably linked to an SV40 promoter.

### Embodiment P19

The plasmid vector of any one of embodiments P1-P18, wherein the nucleic acid encoding the selectable marker is operably linked to an EM7 promoter.

### Embodiment P20

The plasmid vector of any one of embodiments P1-P19, wherein the multiple cloning site comprises the sequence set forth in nucleotides 1427 to 1479 of SEQ ID NO: 2.

### Embodiment P21

The plasmid vector of any one of embodiments P3-P20, wherein the upstream homology arm insertion site comprises the sequence set forth in nucleotides 311 to 336 of SEQ ID NO: 2.

### Embodiment P22

The plasmid vector of any one of embodiments P3-P21, wherein the downstream homology arm insertion site comprises the sequence set forth in nucleotides 2960 to 2985 of SEQ ID NO: 2.

### Embodiment P23

The plasmid vector of any one of embodiments P1-P22, wherein the vector has a nucleotide sequence set forth in SEQ ID NO: 2.

### Embodiment P24

The plasmid vector of embodiment P1, further comprising a transgene inserted at the multiple cloning site.

### Embodiment P25

The plasmid vector of embodiment P24, wherein the transgene encodes a therapeutic protein or a therapeutic RNA.

### Embodiment P26

The plasmid vector of any one of embodiments P3-P25, wherein the length of the upstream homology arm and/or the downstream homology arm is about 500 bases to about 4 kilobases in length.

### Embodiment P27

The plasmid vector of any one of embodiments P1-P26, wherein the transgene nucleic acid ranges from about 5 kb to 300 kb in length.

### Embodiment P28

A method for gene expression comprising transfecting a eukaryotic cell with the vector of any one of embodiments P1-P27, further comprising a transgene inserted at the multiple cloning site, and culturing the cell under conditions suitable for expression of the transgene.

### Embodiment P29

A method for modifying a target genomic locus in a mammalian cell, comprising:

(a) introducing into a mammalian cell:
(i) a nuclease agent that makes a single or double-strand break at or near a target genomic locus, and
(ii) the vector any one of embodiments P1-P27, further comprising a transgene inserted at the multiple cloning site flank an upstream homology arm inserted at the upstream homology arm insertion site and a downstream homology arm inserted at the downstream homology arm; and

(b) selecting a targeted mammalian cell comprising the transgene in the target genomic locus.

### Embodiment P30

The method of embodiment P29, wherein the cell is selected by detection the selectable marker.

### Embodiment P31

The method of embodiments P29 or P30, wherein the mammalian cell is a pluripotent cell.

### Embodiment P32

The method of embodiment P31, wherein the pluripotent cell is an induced pluripotent stem (iPS) cell, embryonic stem (ES) cell, an adult stem cell, a hematopoietic stem cell, a neuronal stem cell.

### Embodiment P33

The method of embodiment P29 or P30, wherein the mammalian cell is a human fibroblast.

### Embodiment P34

The method of embodiment P29 or P30, wherein the mammalian cell is a human cell isolated from a patient having a disease, and wherein the human cell comprises at least one human disease allele in its genome.

### Embodiment P35

The method of embodiment P34, wherein integration of the transgene into the target genomic locus replaces the at least one human disease allele in the genome.

### Embodiment P36

The method of embodiment P29 or P30, wherein the nuclease agent is an expression construct comprising a nucleic acid sequence encoding a nuclease, and wherein the nucleic acid is operably linked to a promoter active in the mammalian cell.

### Embodiment P37

The method of embodiment P36, wherein the nuclease agent is an mRNA encoding a nuclease.

### Embodiment P38

The method of embodiment P36, wherein the nuclease is a zinc finger nuclease (ZFN).

### Embodiment P39

The method of embodiment P36, wherein the nuclease is a Transcription Activator-Like Effector Nuclease (TALEN).

### Embodiment P40

The method of embodiment P36, wherein the nuclease is a meganuclease.

### Embodiment P41

The method of embodiment P36, wherein the nuclease is a Cas9 nuclease.

### Embodiment P42

The method of any one of embodiment P36-P41, wherein a target sequence of the nuclease agent is located in an intron, exon, a promoter, a promoter regulatory region, or an enhancer region in the target genomic locus.

### Embodiment P43

The method of embodiment P42, wherein the target sequence is an AAV1 integration site.

### Embodiment P44

The method of any one of embodiments P36-P43, wherein the length of the upstream homology arm and/or the downstream homology arm is about 500 bases to about 4 kilobases.

### Embodiment P45

The method of any one of embodiments P36-P44, wherein the transgene nucleic acid ranges from about 5 kb to 300 kb in length.

### EMBODIMENTS

### Embodiment 1

A plasmid vector comprising:
(a) a prokaryotic origin of replication;
(b) a eukaryotic promoter suitable for expression of one or more transgenes;
(c) a multiple cloning site for insertion of the one or more transgenes; and
(d) a nucleic acid encoding a selectable marker operably linked to a dual promoter comprising a eukaryotic promoter and a prokaryotic promoter, wherein the selectable marker is suitable for both prokaryotic and eukaryotic selection;
wherein the vector is less than 3.6 kilobases in length.

### Embodiment 2

The plasmid vector of embodiment 1, wherein elements (a) through (d) are arranged sequentially in the 5′ to 3′ direction of the plasmid.

### Embodiment 3

The plasmid vector of embodiment 1 or 2, further comprising an upstream homology arm insertion site located between elements (a) and (b) and a downstream homology arm insertion site.

### Embodiment 4

The plasmid vector of embodiment 3, the downstream homology arm insertion site located after element (d).

### Embodiment 5

The plasmid vector of any one of embodiments 1-4, further comprising a synthetic splice site between elements (b) and (c) that enhances stability of RNA transcribed from the eukaryotic promoter of (b).

### Embodiment 6

The plasmid vector of any one of embodiments 1-5, further comprising poly A sequences following the multiple cloning site of (d).

### Embodiment 7

The plasmid vector of any one of embodiments 1-6, further comprising an additional promotor upstream of the multiple cloning site of (d) for in vitro expression of the one or more transgenes.

### Embodiment 8

The plasmid vector of embodiment 7, wherein the additional promotor for in vitro expression is a T7 promoter.

### Embodiment 9

The plasmid vector of any one of embodiments 1-8, wherein the origin of replication of (a) is selected from the group consisting of pBR322, pMB1, p15A, pACYC184, pACYC177, ColE1, pBR3286, p1, pBR26, pBR313, pBR327, pBR328, pPIGDM1, pPVUI, pF, pSC101 and pC101p-157.

### Embodiment 10

The plasmid vector of embodiment 9, wherein the origin of replication of (a) is pBR322 Ori.

### Embodiment 11

The plasmid vector of any one of embodiments 1-10, wherein the eukaryotic promoter of (b) is selected from the group consisting of a cytomegalovirus (CMV) promoter, the promoter of the Beta-Actin gene from human, mouse, or chicken, the promoter of the Ubiquitin C gene, and the promoter of the Thymidine Kinase gene from Herpes Virus.

### Embodiment 12

The plasmid vector of embodiment 11, wherein the eukaryotic promoter of (b) is a cytomegalovirus (CMV) promoter.

### Embodiment 13

The plasmid vector of any one of embodiments 1-12, wherein the selectable marker is selected from the group consisting of an antibiotic resistance gene, a fluorescent protein, and an enzyme.

### Embodiment 14

The plasmid vector of embodiment 13, wherein the selectable marker is an antibiotic resistance gene.

### Embodiment 15

The plasmid vector of embodiment 13, wherein the selectable marker is blasticidin S deaminase.

### Embodiment 16

The plasmid vector of embodiment 13, wherein the selectable marker is puromycin-N-acetyltransferase.

### Embodiment 17

The plasmid vector of embodiment 13, wherein the selectable marker is neomycin phosphotransferase.

### Embodiment 18

The plasmid vector of embodiment 13, wherein the selectable marker is a fluorescent protein.

### Embodiment 19

The plasmid vector of embodiment 16, wherein the fluorescent protein is a near infrared fluorescent protein.

Embodiment 20, The plasmid vector of any one of embodiments 1-19, wherein the nucleic acid encoding the selectable marker is operably linked to an SV40 promoter.

### Embodiment 21

The plasmid vector of any one of embodiments 1-20, wherein the nucleic acid encoding the selectable marker is operably linked to an EM7 promoter.

### Embodiment 22

The plasmid vector of any one of embodiments 1-21, wherein the multiple cloning site comprises the sequence set forth in nucleotides 1427 to 1479 of SEQ ID NO: 2.

### Embodiment 23

The plasmid vector of any one of embodiments 3-22, wherein the upstream homology arm insertion site comprises the sequence set forth in nucleotides 311 to 336 of SEQ ID NO: 2.

### Embodiment 24

The plasmid vector of any one of embodiments 3-23, wherein the downstream homology arm insertion site comprises the sequence set forth in nucleotides 2960 to 2985 of SEQ ID NO: 2.

### Embodiment 25

The plasmid vector of any one of embodiments 1-24, wherein the vector has a nucleotide sequence set forth in SEQ ID NO: 2.

### Embodiment 26

The plasmid vector of embodiment 1, further comprising a transgene inserted at the multiple cloning site.

### Embodiment 27

The plasmid vector of embodiment 26, wherein the transgene encodes a therapeutic protein or a therapeutic RNA.

### Embodiment 28

The plasmid vector of any one of embodiments 3-27, wherein the length of the upstream homology arm and/or the downstream homology arm is about 500 bases to about 4 kilobases in length.

### Embodiment 29

The plasmid vector of any one of embodiments 1-28, wherein the transgene nucleic acid ranges from about 5 kb to 300 kb in length.

### Embodiment 30

The plasmid vector of any one of embodiments 1-29, wherein the prokaryotic origin of replication is not an F1 origin.

### Embodiment 31

The plasmid vector of any one of embodiments 1-30, wherein the plasmid vector comprises exactly one selectable marker.

### Embodiment 32

A method for gene expression comprising transfecting a eukaryotic cell with the vector of any one of embodiments 1-31, further comprising a transgene inserted at the multiple cloning site, and culturing the cell under conditions suitable for expression of the transgene.

### Embodiment 33

A method for modifying a target genomic locus in a mammalian cell, comprising:
(a) introducing into a mammalian cell:
   (i) a nuclease agent that makes a single or double-strand break at or near a target genomic locus, and
   (ii) the vector any one of embodiments 1-31, further comprising a transgene inserted at the multiple cloning site flank an upstream homology arm inserted at the upstream homology arm insertion site and a downstream homology arm inserted at the downstream homology arm; and
(b) selecting a targeted mammalian cell comprising the transgene in the target genomic locus.

### Embodiment 34

The method of embodiment 33, wherein the cell is selected by detection the selectable marker.

### Embodiment 35

The method of embodiment 33 or 34, wherein the mammalian cell is a pluripotent cell.

### Embodiment 36

The method of embodiment 35, wherein the pluripotent cell is an induced pluripotent stem (iPS) cell, embryonic stem (ES) cell, an adult stem cell, a hematopoietic stem cell, a neuronal stem cell.

### Embodiment 37

The method of embodiment 33 or 34, wherein the mammalian cell is a human fibroblast.

### Embodiment 38

The method of embodiment 33 or 34, wherein the mammalian cell is a human cell isolated from a patient having a disease, and wherein the human cell comprises at least one human disease allele in its genome.

### Embodiment 39

The method of embodiment 38, wherein integration of the transgene into the target genomic locus replaces the at least one human disease allele in the genome.

### Embodiment 40

The method of embodiment 33 or 34, wherein the nuclease agent is an expression construct comprising a nucleic acid sequence encoding a nuclease, and wherein the nucleic acid is operably linked to a promoter active in the mammalian cell.

### Embodiment 41

The method of embodiment 40, wherein the nuclease agent is an mRNA encoding a nuclease.

### Embodiment 42

The method of embodiment 40, wherein the nuclease is a zinc finger nuclease (ZFN).

### Embodiment 43

The method of embodiment 40, wherein the nuclease is a Transcription Activator-Like Effector Nuclease (TALEN).

### Embodiment 44

The method of embodiment 40, wherein the nuclease is a meganuclease.

### Embodiment 45

The method of embodiment 40, wherein the nuclease is a Cas9 nuclease.

### Embodiment 46

The method of any one of embodiments 40-45, wherein a target sequence of the nuclease agent is located in an intron, exon, a promoter, a promoter regulatory region, or an enhancer region in the target genomic locus.

### Embodiment 47

The method of embodiment 46, wherein the target sequence is an AAV1 integration site.

### Embodiment 48

The method of any one of embodiments 40-47, wherein the length of the upstream homology arm and/or the downstream homology arm is about 500 bases to about 4 kilobases.

### Embodiment 49

The method of any one of embodiments 40-48, wherein the transgene nucleic acid ranges from about 5 kb to 300 kb in length.

### Embodiment 50

A kit comprising the vector of any one of embodiments 1-31 and a growth medium comprising an antibiotic.

### Embodiment 51

The kit of embodiments 50, wherein the antibiotic is blasticidin S, puromycin, or neomycin.

### Embodiment 52

The kit of embodiment 50 or 51, wherein the growth medium is a liquid growth medium, a solid growth medium, or a semi-solid growth medium.

### Embodiment 53

The kit of embodiment 50 or 52, wherein the solid growth medium is agar.

### Embodiment 54

The kit of any one of embodiments 50-53, further comprising a first, a second, and a third blend of restriction enzymes.

### Embodiment 55

The kit of embodiment 54, wherein the first blend of restriction enzymes comprises restriction enzymes for restriction sites SwaI and SbfI; wherein the second blend of restriction enzymes comprises restriction enzymes for restriction sites AscI and PmeI; and wherein the third blend of restriction enzymes comprises restriction enzymes for restriction sites PmeI and SwaI.

### Embodiment 56

The kit of any one of embodiments 50-55, further comprising a Type II CRISPR system for genome editing.

### Embodiment 57

The kit of any one of embodiments 50-55, further comprising a TALEN system for genome editing.

### Embodiment 58

The kit of any one of embodiments 50-55, further comprising a zinc-finger nuclease system for genome editing.

### Embodiment 59

A plasmid vector comprising a dual promoter and a single selectable marker that functions in both a eukaryotic and a prokaryotic cell, the vector excluding an additional selectable marker.

1. A plasmid vector comprising:
(a) a prokaryotic origin of replication;
(b) a eukaryotic promoter suitable for expression of one or more transgenes;
(c) a multiple cloning site for insertion of the one or more transgenes; and
(d) a nucleic acid encoding a selectable marker operably linked to a dual promoter comprising a eukaryotic promoter and a prokaryotic promoter, wherein the selectable marker is suitable for both prokaryotic and eukaryotic selection;
wherein the vector is less than 3.6 kilobases in length.

2. The plasmid vector of claim 1, wherein elements (a) through (d) are arranged sequentially in the 5′ to 3′ direction of the plasmid.

3. The plasmid vector of claim 1, further comprising an upstream homology arm insertion site located between elements (a) and (b) and a downstream homology arm insertion site.

4. The plasmid vector of claim 3, wherein the downstream homology arm insertion site located after element (d).

5. The plasmid vector of claim 1, further comprising a synthetic splice site between elements (b) and (c) that enhances stability of RNA transcribed from the eukaryotic promoter of (b).

6. The plasmid vector of claim 1, further comprising poly A sequences following the multiple cloning site of (d).

7. The plasmid vector of claim 1, further comprising an additional promotor upstream of the multiple cloning site of (d) for in vitro expression of the one or more transgenes.

8. (canceled)

9. The plasmid vector of claim 1, wherein the origin of replication of (a) is selected from the group consisting of pBR322, pMB1, p15A, pACYC184, pACYC177, ColE1, pBR3286, p1, pBR26, pBR313, pBR327, pBR328, pPIGDM1, pPVUI, pF, pSC101 and pC101p-157.

10. (canceled)

11. The plasmid vector of claim 1, wherein the eukaryotic promoter of (b) is selected from the group consisting of a cytomegalovirus (CMV) promoter, the promoter of the Beta-Actin gene from human, mouse, or chicken, the promoter of the Ubiquitin C gene, and the promoter of the Thymidine Kinase gene from Herpes Virus.

12. (canceled)

13. The plasmid vector of claim 1, wherein the selectable marker is selected from the group consisting of an antibiotic resistance gene, a fluorescent protein, and an enzyme.

14. (canceled)

15. (canceled)

16. (canceled)

17. (canceled)

18. (canceled)

19. (canceled)

20. The plasmid vector of claim 1, wherein the nucleic acid encoding the selectable marker is operably linked to an SV40 promoter.

21. The plasmid vector of claim 1, wherein the nucleic acid encoding the selectable marker is operably linked to an EM7 promoter.

22. The plasmid vector of claim 1, wherein the multiple cloning site comprises the sequence set forth in nucleotides 1427 to 1479 of SEQ ID NO: 2.

23. The plasmid vector of claim 3, wherein the upstream homology arm insertion site comprises the sequence set forth in nucleotides 311 to 336 of SEQ ID NO: 2.

24. The plasmid vector of claim 2, wherein the downstream homology arm insertion site comprises the sequence set forth in nucleotides 2960 to 2985 of SEQ ID NO: 2.

25. The plasmid vector of claim 1, wherein the vector has a nucleotide sequence set forth in SEQ ID NO: 2.

26. The plasmid vector of claim 1, further comprising a transgene inserted at the multiple cloning site.

27. The plasmid vector of claim 26, wherein the transgene encodes a therapeutic protein or a therapeutic RNA.

28. (canceled)

29. (canceled)

30. (canceled)

31. (canceled)

32. (canceled)

33. A method for modifying a target genomic locus in a mammalian cell, comprising:
(a) introducing into a mammalian cell:
(i) a nuclease agent that makes a single or double-strand break at or near a target genomic locus, and
(ii) the vector of claim 1, further comprising a transgene inserted at the multiple cloning site flanking an upstream homology arm inserted at the upstream homology arm insertion site and a downstream homology arm inserted at the downstream homology arm; and
(b) selecting a targeted mammalian cell comprising the transgene in the target genomic locus.

34. (canceled)

35. (canceled)

36. (canceled)

37. (canceled)

38. (canceled)

39. (canceled)

40. The method of claim 33, wherein the nuclease agent is an expression construct comprising a nucleic acid sequence encoding a nuclease, and wherein the nucleic acid is operably linked to a promoter active in the mammalian cell.

41.-59. (canceled)
